SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2026/0174770 A1
(43) Pub. Date: Jun. 25, 2026

(54) COMBINATION THERAPY FOR TREATING CANCER

(71) Applicant: SCORPION THERAPEUTICS, INC., Indianapolis, IN (US)

(72) Inventors: David ST. JEAN, Jr., Natick, MA (US); Darrin STUART, Boston, MA (US); Leonard BUCKBINDER, Boston, MA (US); Erica JACKSON, Boston, MA (US); Trang Nguyet TIEU, Boston, MA (US); Brendon LADD, Boston, MA (US); Jacob Tyler ALLTUCKER, Boston, MA (US)

(21) Appl. No.: 19/125,531

(22) PCT Filed: Oct. 30, 2023
(86) PCT No.: PCT/US2023/078155

§ 371 Date: Apr. 29, 2025

Related U.S. Application Data
(60) Provisional application No. 63/421,082, filed on Oct. 31, 2022.
(60) Provisional application No. 63/423,383, filed on Nov. 7, 2022.
(60) Provisional application No. 63/488,674, filed on Mar. 6, 2023.
(60) Provisional application No. 63/531,990, filed on Aug. 10, 2023.

(51) Int. Cl. A61K 31/5377

(52) U.S. Cl. CPC A61K 31/5377

(57) ABSTRACT

This disclosure provides compounds of Formula (I), and pharmaceutically acceptable salts thereof, that inhibit phosphatidylinositol 4,5-bisphosphate 3-kinase (PI3K) isoform alpha (PI3Kα) for use in combination with additional therapeutic agents for treating a condition, disease or disorder in which increased (e.g., excessive) PI3Kα activation contributes to the pathology and/or symptoms and/or progression of the condition, disease or disorder (e.g., cancer) in a subject.

### CROSS-REFERENCE TO RELATED APPLICATIONS

This application claims the benefit of the filing date of U.S. Provisional Application No. 63/421,082, filed Oct. 31, 2022, U.S. Provisional Application No. 63/423,383, filed Nov. 7, 2022, U.S. Provisional Application No. 63/488,674, filed Mar. 6, 2023 and U.S. Provisional Application No. 63/531,990, filed Aug. 10, 2023. The contents of each application are incorporated by reference herein in their entirety.

### SEQUENCE LISTING

This application contains a Sequence Listing that has been submitted electronically as an XML file named 50006-0099WO1_ST26_SL.XML. The XML file, created on Oct. 30, 2023, is 2,946 bytes in size. The material in the XML file is hereby incorporated by reference in its entirety.

### TECHNICAL FIELD

This disclosure provides compounds of Formula (I), and pharmaceutically acceptable salts thereof, that inhibit phosphatidylinositol 4,5-bisphosphate 3-kinase (PI3K) isoform alpha (PI3Kα) for use in combination with additional therapeutic agents for treating a condition, disease or disorder in which increased (e.g., excessive) PI3Kα activation contributes to the pathology and/or symptoms and/or progression of the condition, disease or disorder (e.g., cancer) in a subject.

### BACKGROUND

Phosphatidylinositol 4,5-bisphosphate 3-kinase (PI3K) isoform alpha (PI3Kα), encoded by the PIK3CA gene is a part of the PI3K/AKT/TOR signaling network and is altered in several human cancers. Several investigators have demonstrated the role of PI3K/AKT signaling is involved in physiological and pathophysiological functions that drive tumor progression such as metabolism, cell growth, proliferation, angiogenesis and metastasis. See, Fruman, D. A. Cell 2017, 170, 605-635 and Janku, F. et al., Nat. Rev. Clin. Oncol. 2018, 15, 273-291. Pharmacological or genetic suppression of PI3K/AKT/TOR signaling may cause cancer cell death and regression of tumor growth.

The PI3K pathway can be activated via, for example, point mutation(s) of the PIK3CA gene or via inactivation of the phosphatase and tensin homolog (PTEN) gene. Activation of this pathway occurs in approximately 30-50% human cancers and contributes to resistance to various anti-cancer therapies. See, Martini, M. et al., Ann. Med. 2014, 46, 372-383 and Bauer, T. M. et al., Pharmacol. Ther. 2015, 146, 53-60.

### SUMMARY

Provided herein is a method for treating cancer in a subject in need thereof, the method comprising administering to the subject (a) Compound 1, or a pharmaceutically acceptable salt thereof, and (b) one or more additional therapeutic agents.

Provided herein is a method for treating cancer in a subject in need thereof, the method comprising administering to the subject (a) Compound 1, or a pharmaceutically acceptable salt thereof, and (b) one or more independently selected additional therapeutic agents selected from the group consisting of: a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, and a RAF inhibitor, a PIM (Provirus Integration site for Moloney leukemia virus kinase, e.g., PIM1, PIM2, and PIM3) inhibitor, or a combination of any of the foregoing.

Also provided herein is a pharmaceutical composition comprising a compound of Formula (I), or a pharmaceutically acceptable salt thereof, one or more pharmaceutically acceptable excipients, and one or two independently selected additional therapeutic agents.

This disclosure also provides a method for inhibiting PI3Kα in a mammalian cell, the method comprising contacting the mammalian cell with a compound of Formula (I), or a pharmaceutically acceptable salt thereof, and with one or more additional therapeutic agents. In some embodiments, the one or more additional therapeutic agents are a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, a RAF inhibitor, a PIM (e.g., PIM1 and PIM3) inhibitor, or a combination of any of the foregoing.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The phrase “therapeutically effective amount” means an amount of compound that, when administered to a subject in need of such treatment, is sufficient to (i) treat a PI3Kα protein-associated disease or disorder, (ii) attenuate, ameliorate, or eliminate one or more symptoms of the particular disease, condition, or disorder, or (iii) delay the onset of one or more symptoms of the particular disease, condition, or disorder described herein. When used in reference to treatment with more than one therapeutic agent, each agent can independently be administered in a therapeutically effective amount (e.g., an amount that would be therapeutically effective as a monotherapy) or the one or more therapeutic agents can together be a therapeutically effective amount (e.g., a therapeutically effective amount of a combination therapy) for treating the indicated disease or disorder. In other words, the amount of the individual components in a therapeutically effective amount of a combination therapy can, independently, be administered (in the combination) at less than a therapeutically effective amount when administered as a monotherapy.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein from with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

As used herein, the “subject” refers to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the cancer to be treated.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission (partial or complete) for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease. “Treatment” can also mean prolonging survival as compared to expected survival if not receiving treatment.

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Metastatic brain cancer refers to cancer in the brain, i.e., cancer which originated in a tissue other than the brain and has metastasized to the brain. In some embodiments described herein, the subject has metastatic brain cancer and/or metastatic spinal cord cancer.

The term “tumor growth inhibition (TGI) index” refers to a value used to represent the degree to which an agent (e.g., Compound 1, or a pharmaceutically acceptable salt thereof, alone or in combination with one or more additional therapeutic agents as described herein) inhibits the growth of a tumor when compared to an untreated control, or a control treated with a monotherapy being evaluated as part of a combination therapy. The TGI index is calculated for a particular time point (e.g., a specific number of days into an experiment or clinical trial) according to the following formula:

$$TGI = 1 - ( \frac{{Volume}_{treated ( Tx Day X )} - {Volume}_{treated ( Tx Day 0 )}}{{Volume}_{control ( Tx Day X )} - {Volume}_{control ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The compounds of “Formula (I)”, or a pharmaceutically acceptable salt thereof, refers to:

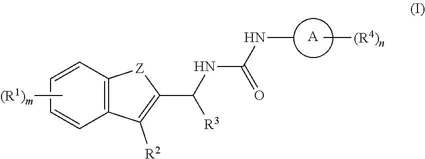

or a pharmaceutically acceptable salt thereof, wherein:
Z is O or NR^{x};
R^{x} is hydrogen, C1-C6 alkyl, or C3-C6 cycloalkyl;
each R¹ is independently selected from halogen, hydroxyl, cyano, C1-C6 alkyl optionally substituted with hydroxyl, and C3-C6 cycloalkyl;
m is 0, 1, 2, or 3;
R² is halogen, hydroxyl, C1-C6 alkyl optionally substituted with hydroxyl, C1-C6 haloalkyl, C3-C6 cycloalkyl optionally substituted with 1 or 2 fluoro;
R³ is a C1-C6 alkyl, a C1-C6 haloalkyl, or a C3-C6 cycloalkyl optionally substituted with 1 or 2 substituents independently selected from fluoro and C1-C6 alkyl;
Ring A is a 6-10 membered aryl, a C3-C8 cycloalkyl, a 5-10 membered heteroaryl, or a 4-10 membered heterocyclyl;
each R⁴ is independently selected from the group consisting of:
(i) halogen,
(ii) C1-C6 alkyl optionally substituted with 1 or 2 hydroxyl or —NR^{A}R^{B},
(iii) C1-C6 alkoxy optionally substituted with 1-2 substituents independently selected from hydroxyl and C3-C6 cycloalkyl,
(iv) C1-C6 haloalkyl,
(v) hydroxyl,
(vi) cyano,
(vii) —CO₂H,
(viii) —NR^{A}R^{B},
(ix)=NR^{A2},
(x) —C(═O)NR^{C}R^{D},
(xi) —SO₂(NR^{E}R^{F}),
(xii) —SO₂(C1-C6 alkyl),
(xiii) —S(═O)(═NH)(C1-C6 alkyl),
(xiv) —C(═O)(C1-C6 alkyl),
(xv) —CO₂(C1-C6 alkyl),
(xvi) 5-6 membered heteroaryl optionally substituted with C1-C6 alkyl,
(xvii) 3-9 membered heterocyclyl optionally substituted with 1 or 2 independently selected R^{G}, and
(xviii) 3-6 membered cycloalkyl optionally substituted with 1 or 2 independently selected R^{G};
n is 0, 1, or 2;
each R^{A}, R^{A1}, R^{B}, R^{B1}, R^{C}, R^{C1}, R^{D}, R^{D1}, R^{E}, and R^{F} is independently
(i) hydrogen,
(ii) hydroxyl,
(iii) 4-6 membered heterocyclyl,
(iv) C1-C6 haloalkyl,
(v) —C(═O)(C1-C6 alkyl),
(vi) —C(═O)O(C1-C6 alkyl),
(vii) —SO₂(C1-C6 alkyl),
(viii) 3-6 membered cycloalkyl optionally substituted with hydroxyl, or
(ix) C1-C6 alkyl optionally substituted with 1-2 substituents independently selected from hydroxyl, —C(═O)NR^{B2}R^{C2}, 5-6 membered heteroaryl, 3-6 membered cycloalkyl, —SO₂(C1-C6 alkyl), —CO₂H, and —SO₂(NH₂); or
R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy;
each R^{A2}, R^{B2}, and R^{C2} is independently hydrogen or C1-C6 alkyl; and
each R^{G} is independently selected from the group consisting of: fluoro, cyano, hydroxyl, C1-C6 alkyl optionally substituted with hydroxyl, C1-C6 alkoxy, —NR^{A1}R^{B1}, =NR^{A2}, —C(═O)NR^{C1}R^{D1}, —CO₂(C1-C6 alkyl), C1-C6 haloalkyl, C3-C6 cycloalkyl, C1-C6 haloalkoxy, —SO₂(C1-C6 alkyl), and —CO₂H.

“Compound 1”, or a pharmaceutically acceptable salt thereof, refers to (R)-1-(2-aminopyrimidin-5-yl)-3-(1-(5,7-difluoro-3-methylbenzofuran-2-yl)-2,2,2-trifluoroethyl)urea, or a pharmaceutically acceptable salt thereof, having the structure:

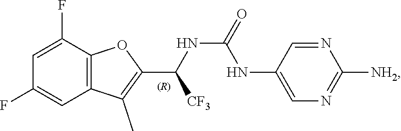

or a pharmaceutically acceptable salt thereof.

The details of one or more embodiments of this disclosure are set forth in the accompanying drawings and the description below. Other features and advantages of the present disclosure will be apparent from the description and drawings, and from the claims.

### BRIEF DESCRIPTION OF THE FIGURES

FIG. 1A shows an isobologram of fulvestrant vs. Compound 1. FIG. 1B shows an isobologram of lapatinib vs. Compound 1. FIG. 1C shows an isobologram of abemaciclib vs. Compound 1. FIG. 1D is a schematic of the RAS pathway and potential inhibitors to the MEK1/2 pathway genes. FIG. 1E shows the results of a Profiling Relative Inhibition Simultaneously in Mixtures (PRISM) screen of drugs against wild type MAPK and mutant MAPK. FIG. 1F shows an isobologram of tramenitinib vs. Compound 1. FIG. 1G shows the growth inhibition of MSI-H colon cancer cells when treated with Compound 1 and/or binimetinib.

FIG. 2A shows that Compound 1 decreases tumor volume in a PDX model, and that the decrease in tumor volume is more pronounced when Compound 1 is administered in combination with fulvestrant. PDX Model 1 measures PIK3CA^{H1047R}, ER⁺ tumor volume with the circle representing vehicle, diamonds representing fulvestrant 5 mg SC QW, triangles representing Compound 1 at 100 mg/kg PO QD, and the square representing Compound 1 at 100 mg/kg+fulvestrant. FIG. 2B shows tumor volume before and after treatment with Compound 1 at 100 mg/kg+fulvestrant. FIG. 2C shows tumor volume before and after treatment with Compound 1 at 100 mg/kg PO QD.

FIGS. 3A-3B show that Compound 1 decreases tumor volume in a PDX model, and that the decrease in tumor volume is more pronounced when Compound 1 is administered in combination with palbociclib. PDX model 2 measures PIK3CA^{H1047WR/R08H}, ER⁺/HER2⁺ tumor volume with the circle representing vehicle, triangles representing Compound 1 at 100 mg/kg PO QD, the green circle represents palbociclib PO QD, and the square representing Compound 1 at 100 mg/kg+palbociclib at 50 mg/kg. FIG. 3B shows PDX model 3: PIK3CA^{H1047R}, ER⁺/HER2⁺ tumor change in volume with the circle representing vehicle, triangles representing Compound 1 at 100 mg/kg PO QD, the green circle represents palbociclib PO QD, and the square representing Compound 1 at 100 mg/kg+palbociclib at 50 mg/kg.

FIGS. 4A-4G show Compound 1 combination therapies in ER⁺ breast cancer models with fulvestrant and/or palbociclib. FIG. 4A shows T47D xenograft tumors were established in female NSG mice. When tumors reached approximately 200 mm³, mice were randomized and treated as indicated. Data represent percent change in tumor volume of individual tumors on day 20 relative to randomization. FIGS. 4B and 4C show NSG mice bearing T47D tumors were given a single dose of vehicle or compound(s) as indicated. Tumors were harvested 4 hours after treatment with Compound 1 and 24 hours post-treatment with fulvestrant for (4B) western blot of pAKT (S473) and S6 (S240/244) and (4C) IHC of AKT and S6, and pS6 and pAKT (S473) groups were blotted and analyzed together. All samples for a given protein were run on the same ge. A vinculin analysis is shown. For clarity, images were cropped to remove other compounds. 20× IHC images are shown with a 200 μm scale bar. FIG. 4D shows ST1056 xenograft tumors were established in BALB/c nude mice. Mice were randomized to treatment groups as indicated at approximately 260 mm³. Mice were treated for 94 days or until removal from the study due to tumor volume. After 94 days, treatment ended, and tumors were monitored for regrowth. FIG. 4E shows percent change in body weight for FIG. 4D. FIGS. 4F and 4G show percent change in body weight (top) and tumor volume measurements (bottom) over time from the T47D efficacy study represented in FIG. 4A (N=9).

FIG. 5A shows inhibition of enzyme activities of WT PI3Kα as well as kinase-domain and helical domain mutant proteins by alpelisib and Compound 1 showing the geometric mean and standard deviation. FIG. 5B shows surface plasmon resonance (SPR) sensograms for Compound 1 (top graph) and duvelisib (bottom graph) binding to WT, H1047R, and E545K-mutant proteins are shown in the table below the sensorgrams. FIG. 5C shows the 2.9 Å X-ray structure of PI3Kα (p110=purple, p85=orange) with GDC-0077 (red spheres) bound in the ATP-binding site and Compound 1 (green spheres) bound in the allosteric site. FIG. 5D shows arrows that indicate Ca (spheres) movements of a ≥3 Å between aligned H1047R (teal=PDB 3HHm) and the Compound 1-bound structure (purple). 3HHM lacks residues 941-952 for direct comparison. FIG. 5E shows a detailed view of bound Compound 1 and labeled residues significantly contribute to compound binding. FIG. 5F shows the molecular structure of Compound 1. 5G shows Compound 1 has broad kinome selectivity. Top panel, a table summarizing Compound 1 kinome profiling compared with published alpelisib data. AurB kinase as the only off-target with an IC₅₀<10 mM (˜1658 nM). Bottom panel, dose-dependent inhibition of phospho-H3 (Ser10) observed 1-hour post treatment with AurB kinase inhibitor, barasertib. There was no significant inhibition of AurB observed with Compound 1 treatment.

FIGS. 6A-6F show PI3Kα-mutant selectivity profiling of Compound 1 in cellular assays. FIG. 6A shows a correlation plot of alpelisib and Compound 1 comparing pAKT IC₅₀ at 1 hour and viability (CTGlo) GI₅₀ at 72 hours across the panel of cell lines. FIG. 6B shows pAKT inhibition dose response curves (HTRF assay). FIG. 6C shows a correlation plot comparing alpelisib and Compound 1 potency (pAKT HTRF assay) in a panel of kinase domain mutant cell lines (orange dots) and the WT PI3Kα SKBR3 (black dot). FIG. 6D shows Compound 1 and alpelisib AUC from the Broad Institute's PRISM screen (theprismlab.org) across the CCLE and grouped by PIK3CA mutational status. FIG. 6E shows two-hour glucose uptake in primary human adipocytes as indicated in the bar graph (percent vehicle response). Seventy two-hour viability data in H1047R-PI3Kα-mutant T47D cell line in the orange dose response curve is overlayed for comparison.

FIGS. 7A-7D show the effect of Compound 1 and alpelisib on glucose homeostasis. FIG. 7A shows tumor-naïve female BALB/c mice were dosed for 5 days as indicated (n=5/group) and subjected to an insulin tolerance test (ITT). On day 5, animals were fasted for 6 hours and dosed as indicated 1 hour prior to the end of the fast (−1 hour). At T=0, animals were dosed with 0.75 U/kg intraperitoneal insulin. Blood glucose levels were monitored over time with the group mean and standard error mean shown. FIG. 7B shows AUC calculated from FIG. 7A. The AUC of each treatment group was compared with the vehicle group using ordinary one-way ANOVA and Dunnet's multiple comparisons tests. FIG. 7C shows oral glucose tolerance test performed similar to the ITT of FIG. 7A, except at T=0, the mice were dosed with 2 g/kg glucose and blood glucose was monitored over time with group mean and standard error mean indicated. FIG. 7D shows AUC calculated from data shown in FIG. 7C (calculations analyzed as done in FIG. 7B).

FIGS. 8A-8J show efficacy and pharmacodynamics profiling of Compound 1 versus alpelisib in CAL33 xenograft tumors. FIGS. 8A-8B show Cal33 xenograft tumors were established in female BALB/c nude mice. Animals were randomized into treatment groups (n=6) at approximately 160 mm³ and treated as indicated. Tumor (8A) and BW (8B) were measured two times a week with group mean and standard error mean shown. FIGS. 8C-8D show mice bearing Cal33 tumors were treated as indicated for 3 days, and serum insulin (8C) and blood glucose (8D) were measured with individual values, group mean, and standard error mean shown with each treatment group compared to vehicle group using ordinary one-way ANOVA and Dunnet's multiple comparisons tests. FIG. 8E shows pAKT (S473) measured by western blot in all Cal33 tumors from mice in the 28-day efficacy study (30- and 100-mg dose groups) and 3 day PK/PD study (30-, 100-, and 300-mg/kg dose groups). pAKT (S473) and total AKT was measured in Cal33 tumors by Western blot. Th normalized pAKT (S473) levels were plotted against unbound concentration (blue). The in vitro pAKT dose responses in Cal33 has been added for reference. FIG. 8F shows pAKT levels in Cal33 tumors showing only Compound 1 (100 mg/kg) and alpelisib (50 mg/kg) over time. FIG. 8G shows pAKT (S473) measured by western blot from the gastrocnemius from the mice of FIG. 8E-8F. Significance was measured using ordinary one-way ANOVA and Dunnet's multiple comparisons tests. FIG. 8H shows N=4 mice fasted for 4 hours then administered vehicle, Compound 1 (100 mg/kg), or alpelisib (50 mg/kg). One hour later the mice were administered [U-¹³C]-glucose orally. After 30 minutes, the tissues were collected and analyzed by MS. The abundance of labeled [M+4] succinic acid (S), fumaric acid (F), and malic acid (M) in tumor and gastrocnemius are shown. Individual values, group mean, and standard error mean are shown with each treatment group compared with the vehicle group using two-way ANOVA and Holm-Šídák post-test. FIG. 8I shows plasma insulin measured immediately before and 30 minutes after labeled-glucose administration. FIG. 8J shows measurements from the plasma from mice represented in FIG. 8H. The isotopolouge state represents the counts of labeled U-¹³C carbons on glucose. Animals from the 0-hour group were not dosed with glucose; samples were collected at the time glucose was administered to the mice collected 30-minutes post [U-¹³C]-glucose. Bars represent SD.

FIG. 9 shows mice bearing Cal33 tumors were treated as indicated for 3 days, and serum insulin were measured with individual values, group mean, and standard error mean shown with each treatment group compared to vehicle group using ordinary one-way ANOVA and Dunnet's multiple comparisons tests.

FIG. 10 p-AKT and AKT WB Analysis in efficacy groups. Tumor samples were analyzed for total-AKT and p-AKT by Western blot. For every treatment group analyzed the corresponding vehicle control samples were processed in parallel and run on the same gel and WB to minimize variation. The ratio of p-AKT:AKT for the time-matched vehicle control was defined as 100% and all treated samples were expressed relative to that and illustrated in the corresponding scatter plots with the indicated mean and SEM.

FIGS. 11A-11I show metabolically sparing doses of Compound 1 are similarly efficacious to a high dose of alpelisib across PI3Kα-mutant tumor xenografts. FIG. 11A-11C show tumor volumes over time from the PDX models (N=3/group) harboring PI3Kα mutations in the kinase domain (ST1056-H1047R; 11C), kinase and helical domains (ST1799-E542K/H1065L; 11B), and helical domain (ST2652-E545K; 11A) treated with either vehicle, Compound 1, or alpelisib. Statistical significance was calculated using two-way ANOVAand Dunnett's multiple comparison tests. End-of-study tumors were harvested 4 hours after the final dose and pAKT (S473) was analyzed by Western blot. One of three tumors from model ST1799 were used to determine that the E542K/H1065L PI3Kα mutations are in cis by IsoSeq. AKT and pAKT (S473) were blotted separately and a vinculin analysis is shown. FIG. 11D shows N=9 for NCI-H1048, Detroit562, and GP2D models; n=6 for model HCC1954. Percent change in body weight changes in PDX efficacy studies represented in FIGS. 11A-11C. N=3 in each model; bars represent SEM. FIG. 11E shows final tumor volume percent change represented for GP2D, Detroit562, NCI-H1048, and HCC 1954 CDX tumors. FIGS. 11F-11I show tumor volume measurements (left) and percentage change in body weight (right) over time from the CDX efficacy studies in FIG. 11E.

FIG. 12 shows Compound 1 exposure versus time relationship following a single oral dose of Compound 1 at 30, 100, or 300 mg/kg in CD1 mice. Total exposure and IC₈₀ potency are corrected for both mouse PPB (fu=0.033) and assay media binding (10% FBS fu=0.26), respectively.

### DETAILED DESCRIPTION

This disclosure provides compounds of Formula (I), and pharmaceutically acceptable salts thereof, that inhibit PI3Kα for use in combination with additional therapeutic agents for treating a condition, disease or disorder in which increased PI3Kα activation contributes to the pathology and/or symptoms and/or progression of the condition, disease or disorder (e.g., cancer) in a subject.

As described herein, selective targeting of mutant PI3Kα with Compound 1 lacked the systemic metabolic dysfunction caused by alpelisib. Compound 1 preserved glucose uptake in human adipocytes and did not induce systemic insulin resistance in vivo, as demonstrated by insulin tolerance testing, the lack of insulin spikes following chronic Compound 1 administration in six independent CDX studies and in OGTT, as compared with alpelisib. The selective targeting of mutant PI3Kα was further shown by differential effects of Compound 1 on pAKT/AKT suppression in tumor and muscle, and by assessing glucose oxidation in these tissues with an isotope-labeled glucose OGTT. These data indicate a superior metabolic safety profile of Compound 1 relative to alpelisib, which may enhance efficacy as a result of blunting the counter regulatory insulin spikes that diminish efficacy in preclinical studies (Hopkins et al. Nature 2018; 560(7719):499-503).

Compound 1 monotherapy was studied in a panel of ten CDX and PDX tumors comprising primarily breast and HNSCC tumors, with one example each for colon and lung cancers. This panel represented prevalent PI3Kα-mutated cancer types for which there is a high unmet need for improved treatment options. Compound 1 100 mg/kg QD demonstrated robust efficacy in xenograft models that was similar or superior to high-dose alpelisib, with mouse alpelisib exposure that exceeded patient exposure by approximately 2-fold (based on AUC24 h with 50 mg dose). Compound 1 was equally efficacious in GP2D-mutant colon carcinoma xenografts bearing the H1047L variant, the second-most prevalent kinase domain mutation after H1047R. Importantly, treatment was highly efficacious in ST2652 and ST1799 PDX tumors harboring E545K and E542K mutations, the second- and third-most common mutation hotspots, respectively. Finally, NCIH1048 and ST1799 tumors both harbored secondary PI3Kα mutations, which occur in about 10% of primary tumor samples, with both tumors responding favorably to Compound 1 treatment.

CDK4/6 inhibitors and anti-estrogen therapies are important standard-of-care treatments for ER⁺ breast cancer. The clear superiority of alpelisib and fulvestrant combination therapy versus fulvestrant monotherapy highlights why it is critical to test PI3Kα inhibitors in combination in preclinical models. In the benchmark T47D ER⁺HER2⁻ BrCa CDX model, fulvestrant monotherapy provided a moderate level of tumor growth control, whereas low-dose Compound 1 monotherapy and high-dose alpelisib resulted in tumor stasis. The combination of fulvestrant with low-dose was superior to low-dose Compound 1 monotherapy, with regressions in the majority of animals. The higher dose of Compound 1 resulted in deep tumor regressions with or without fulvestrant combination therapy. Palbociclib, fulvestrant, and Compound 1 monotherapies as well as all combinations, were studied, including triple therapy in an aggressive BrCa PDX model (ST1056). Compound 1 monotherapy provided robust and durable responses in this model, far superior to fulvestrant or palbociclib monotherapy or their combination. However, the combination of fulvestrant and Compound 1 provided exceptional tumor growth control, with regressions that were sustained in every animal for over 90 days of treatment and preserved for weeks after dosing was stopped up to study end. While palbociclib demonstrated limited efficacy as a monotherapy or in combination with Compound 1 in this PDX model, triple combination therapy with Compound 1 and fulvestrant was well tolerated in mice over 90 treatment days. In contrast, the triple combination of alpelisib with ribociclib and fulvestrant resulted in elevated hepatobiliary toxicity, as well as increased incidence and severity of rash, and was not tolerated in patients (Tolaney S M, et al. Clin Cancer Res 2021; 27(2):418-28). Of note, the toxicity from triple combination may have been due to interfering drug metabolism, rather than intolerance to the combined mechanisms of action; given that inavolisib, another non-mutant-selective PI3Kα inhibitor, appears to be tolerated in triple combination (Bedard P L, et al. J Clin Oncol 2022; 40(16_suppl):1052) and is advancing in phase 3 studies with CDK4/6 inhibitor and fulvestrant (NCT04191499).

### Methods of Treatment

### Indications

Provided herein are methods of treating or preventing diseases or disorders associated with dysregulation of a PIK3CA gene, a PI3Kα protein, or the expression or activity or level of any of the same (i.e., a PI3Kα-associated disease or disorder), such as PIK3CA-related overgrowth syndromes ((PROS), see, e.g., Venot, et al., Nature, 558, 540-546 (2018)), brain disorders (e.g., as macrocephaly-capillary malformation (MCAP) and hemimegaloencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3Kα-associated cancer).

In some embodiments, the compounds provided herein can exhibit potent and selective inhibition of PI3Kα. For example, the compounds provided herein can bind to the helical phosphatidylinositol kinase homology domain catalytic domain of PI3Kα. In some embodiments, the compounds provided herein can exhibit nanomolar potency against a PI3Kα kinase including one or more mutations, for example, the mutations in Tables 1 and 2.

In some embodiments, the compounds provided herein can exhibit potent and selective inhibition of mutant PI3Kα. For example, the compounds provided herein can bind to an alloseric site in the kinase domain. In some embodiments, the compounds provided herein can exhibit nanomolar potency against a PI3Kα protein including an activating mutation, with minimal activity against related kinases (e.g., wild type PI3Kα). Inhibition of wild type PI3Kα can cause undesirable side effects (e.g., hyperglycemia and skin rashes) that can impact quality of life and compliance. In some cases, the inhibition of wild type PI3Kα can lead to dose limiting toxicities. See, e.g., Hanker, et al., Cancer Disc. 2019, 9, 4, 482-491. Mutant-selective inhibitors may reduce the risk of such dose limiting toxicities, including hyperglycemia, observed with inhibitors of wild type PI3Kα.

In some embodiments, Compound 1, or a pharmaceutically acceptable salt thereof, selectively targets PI3Kα. For example, Compound 1, or a pharmaceutically acceptable salt thereof, selectively targets PI3Kα over another kinase or non-kinase target.

In some embodiments, Compound 1, or a pharmaceutically acceptable salt thereof, can exhibit greater inhibition of PI3Kα containing one or more mutations as described herein (e.g., one or more mutations as described in Table 1 or Table 2) relative to inhibition of wild type PI3Kα. In some embodiments, Compound 1, or a pharmaceutically acceptable salt thereof can exhibit at least 2-fold, 3-fold, 5-fold, 10-fold, 25-fold, 50-fold or 100-fold greater inhibition of PI3Kα containing one or more mutations as described herein relative to inhibition of wild type PI3Kα. In some embodiments, Compound 1, or a pharmaceutically acceptable salt thereof, can exhibit up to 1000-fold greater inhibition of PI3Kα containing one or more mutations as described herein relative to inhibition of wild type PI3Kα. In some embodiments, Compound 1, or a pharmaceutically acceptable salt thereof, can exhibit up to 10000-fold greater inhibition of PI3Kα having a combination of mutations described herein relative to inhibition of wild type PI3Kα.

In some embodiments, Compound 1, or a pharmaceutically acceptable salt thereof, selectively targets mutant PI3Kα (e.g., PI3Kα^{H1047R}) over wild-type PI3Kα. Compound 1, or pharmaceutically acceptable salts thereof, is useful for treating diseases and disorders which can be treated with a PI3Kα inhibitor, such as PI3Kα-associated diseases and disorders, e.g., PIK3CA-related overgrowth syndromes (PROS) and proliferative disorders such as cancers, including hematological cancers and solid tumors (e.g., advanced or metastatic solid tumors).

In some embodiments, the subject has been identified or diagnosed as having a cancer with a dysregulation of a PIK3CA gene, a PI3Kα protein, or expression or activity, or level of any of the same (a PI3Kα-associated cancer) (e.g., as determined using a regulatory agency-approved, e.g., FDA-approved, assay or kit). In some embodiments, the subject has a tumor that is positive for a dysregulation of a PIK3CA gene, a PI3Kα protein, or expression or activity, or level of any of the same (e.g., as determined using a regulatory agency-approved assay or kit). For example, the subject has a tumor that is positive for a mutation as described in Table 1 or Table 2. The subject can be a subject with a tumor(s) that is positive for a dysregulation of a PIK3CA gene, a PI3Kα protein, or expression or activity, or level of any of the same (e.g., identified as positive using a regulatory agency-approved, e.g., FDA-approved, assay or kit). The subject can be a subject whose tumors have a dysregulation of a PIK3CA gene, a PI3Kα protein, or expression or activity, or a level of the same (e.g., where the tumor is identified as such using a regulatory agency-approved, e.g., FDA-approved, kit or assay). In some embodiments, the subject is suspected of having a PI3Kα-associated cancer. In some embodiments, the subject has a clinical record indicating that the subject has a tumor that has a dysregulation of a PIK3CA gene, a PI3Kα protein, or expression or activity, or level of any of the same (and optionally the clinical record indicates that the subject should be treated with any of the compositions provided herein).

In some embodiments, the subject is a pediatric subject. See, e.g., Berhman R E, et al., Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery and First, Pediatric Medicine, 2nd Ed. Baltimore: Williams & Wilkins; 1994.

In certain embodiments, compounds of Formula (I), or pharmaceutically acceptable salts thereof, are useful for preventing diseases and disorders as defined herein (for example, PIK3CA-related overgrowth syndromes (PROS) and cancer). The term “preventing” as used herein means to delay the onset, recurrence or spread, in whole or in part, of the disease or condition as described herein, or a symptom thereof.

The term “PI3Kα-associated disease or disorder” as used herein refers to diseases or disorders associated with or having a dysregulation of a PIK3CA gene, a PI3Kα protein, or the expression or activity or level of any (e.g., one or more) of the same (e.g., any of the types of dysregulation of a PIK3CA gene, or a PI3Kα protein, or the expression or activity or level of any of the same described herein). Non-limiting examples of a PI3Kα-associated disease or disorder include, for example, PIK3CA-related overgrowth syndromes (PROS), brain disorders (e.g., as macrocephaly-capillary malformation (MCAP) and hemimegaloencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3Kα-associated cancer).

The term “PI3Kα-associated cancer” as used herein refers to cancers associated with or having a dysregulation of a PIK3CA gene, a PI3Kα protein, or expression or activity, or level of any of the same. Non-limiting examples of PI3Kα-associated cancer are described herein.

The phrase “dysregulation of a PIK3CA gene, a PI3Kα protein, or the expression or activity or level of any of the same” refers to a genetic mutation (e.g., a mutation in a PIK3CA gene that results in the expression of a PI3Kα that includes a deletion of at least one amino acid as compared to a wild type PI3Kα, a mutation in a PIK3CA gene that results in the expression of PI3Kα with one or more point mutations as compared to a wild type PI3Kα, a mutation in a PIK3CA gene that results in the expression of PI3Kα with at least one inserted amino acid as compared to a wild type PI3Kα, a gene duplication that results in an increased level of PI3Kα in a cell, or a mutation in a regulatory sequence (e.g., a promoter and/or enhancer) that results in an increased level of PI3Kα in a cell), an alternative spliced version of PI3Kα mRNA that results in PI3Kα having a deletion of at least one amino acid in the PI3Kα as compared to the wild type PI3Kα), or increased expression (e.g., increased levels) of a wild type PI3Kα in a mammalian cell due to aberrant cell signaling and/or dysregulated autocrine/paracrine signaling (e.g., as compared to a control non-cancerous cell). As another example, a dysregulation of a PIK3CA gene, a PI3Kα protein, or expression or activity, or level of any of the same, can be a mutation in a PIK3CA gene that encodes a PI3Kα that is constitutively active or has increased activity as compared to a protein encoded by a PIK3CA gene that does not include the mutation. Non-limiting examples of PI3Kα point mutations/substitutions/insertions/deletions are described in Table 1 and Table 2.

The term “activating mutation” in reference to PI3Kα describes a mutation in a PIK3CA gene that results in the expression of PI3Kα that has an increased kinase activity, e.g., as compared to a wild type PI3Kα, e.g., when assayed under identical conditions. For example, an activating mutation can be a mutation in a PIK3CA gene that results in the expression of a PI3Kα that has one or more (e.g., two, three, four, five, six, seven, eight, nine, or ten) amino acid substitutions (e.g., any combination of any of the amino acid substitutions described herein) that has increased kinase activity, e.g., as compared to a wild type a PI3Kα, e.g., when assayed under identical conditions. In another example, an activating mutation can be a mutation in a PIK3CA that results in the expression of a PI3Kα that has one or more (e.g., two, three, four, five, six, seven, eight, nine, or ten) amino acids deleted, e.g., as compared to a wild type PI3Kα, e.g., when assayed under identical conditions. In another example, an activating mutation can be a mutation in a PIK3CA gene that results in the expression of a PI3Kα that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type PI3Kα, e.g., the exemplary wild type PI3Kα described herein, e.g., when assayed under identical conditions. Additional examples of activating mutations are known in the art.

The term “wild type” or “wild-type” describes a nucleic acid (e.g., a PIK3CA gene or a PI3Kα mRNA) or protein (e.g., a PI3Kα) sequence that is typically found in a subject that does not have a disease or disorder related to the reference nucleic acid or protein.

The term “wild type PI3Kα” or “wild-type PI3Kα” describes a normal PI3Kα nucleic acid (e.g., a PIK3CA or PI3Kα mRNA) or protein that is found in a subject that does not have a PI3Kα-associated disease, e.g., a PI3Kα-associated cancer (and optionally also does not have an increased risk of developing a PI3Kα-associated disease and/or is not suspected of having a PI3Kα-associated disease), or is found in a cell or tissue from a subject that does not have a PI3Kα-associated disease, e.g., a PI3Kα-associated cancer (and optionally also does not have an increased risk of developing a PI3Kα-associated disease and/or is not suspected of having a PI3Kα-associated disease).

Provided herein is a method of treating cancer (e.g., a PI3Kα-associated cancer) in a subject in need thereof, comprising administering to the subject a compound of Formula (I), or a pharmaceutically acceptable salt thereof, (e.g., Compound 1, or a pharmaceutically acceptable salt thereof), or a pharmaceutical composition thereof, and one or more independently selected additional therapeutic agents, as described herein. For example, provided herein are methods for treating PI3Kα-associated cancer in a subject in need thereof, comprising a) detecting a dysregulation of PIK3CA gene, a PI3Kα protein, or the expression or activity or level of any of the same in a sample from the subject; and b) administering Compound 1, or a pharmaceutically acceptable salt thereof and one or more independently selected additional therapeutic agents. In some embodiments, the dysregulation of a PIK3CA gene, a PI3Kα protein, or the expression or activity or level of any of the same includes one or more a PI3Kα protein substitutions/point mutations/insertions. Non-limiting examples of PI3Kα protein substitutions/insertions/deletions are described in Table 1 and Table 2.

Some embodiments provide a method of treating cancer in a subject in need thereof, comprising administering to the subject Compound 1, or a pharmaceutically acceptable salt thereof, and one or more independently selected additional therapeutic agents.

Some embodiments provide a method of treating cancer in a subject in need thereof, comprising administering to the subject a combination therapy comprising Compound 1, or a pharmaceutically acceptable salt thereof, and one or more independently selected additional therapeutic agents.

Some embodiments provide a method of treating cancer in a subject in need thereof, comprising:
(a) testing or having tested a subject to determine that the subject has a PI3Kα-associated cancer, and
(b) administering to the subject Compound 1, or a pharmaceutically acceptable salt thereof, and one or more independently selected additional therapeutic agents.

Some embodiments provide a method of treating cancer in a subject in need thereof, comprising:
(a) determining that the subject has a PI3Kα-associated cancer; and
(b) administering to the subject Compound 1, or a pharmaceutically acceptable salt thereof, and one or more independently selected additional therapeutic agents.

Some embodiments provide a method of treating cancer in a subject in need thereof, comprising:
(a) determining that the subject has a PI3Kα-associated cancer; and
(b) administering to the subject a combination therapy comprising Compound 1, or a pharmaceutically acceptable salt thereof, and one or more independently selected additional therapeutic agents.

Some embodiments provide a method of treating cancer in a subject previously determined to have a PI3Kα-associated cancer, comprising administering to the subject Compound 1, or a pharmaceutically acceptable salt thereof, and one or more independently selected additional therapeutic agents.

Some embodiments provide a method of treating cancer in a subject previously determined to have a PI3Kα-associated cancer, comprising administering to the subject of a combination therapy comprising Compound 1, or a pharmaceutically acceptable salt thereof, and one or more additional therapeutic agents.

Some embodiments provide a method of treating cancer in a subject in need thereof, comprising administering to the subject
(a) Compound 1, or a pharmaceutically acceptable salt thereof, and
(b) one or more independently selected additional therapeutic agents selected from the group consisting of: a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, and a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

Some embodiments provide a method of treating cancer in a subject in need thereof, comprising administering to the subject a combination therapy comprising:
(a) Compound 1, or a pharmaceutically acceptable salt thereof, and
(b) one or more independently selected additional therapeutic agents selected from the group consisting of: a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, and a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

Some embodiments provide a method of treating cancer in a subject in need thereof, comprising:
(a) testing or having tested a subject to determine that the subject has a PI3Kα-associated cancer, and
(b) administering to the subject Compound 1, or a pharmaceutically acceptable salt thereof, and a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

Some embodiments provide a method of treating cancer in a subject in need thereof, comprising:
(a) determining that the subject has a PI3Kα-associated cancer; and
(b) administering to the subject Compound 1, or a pharmaceutically acceptable salt thereof, and a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

Some embodiments provide a method of treating cancer in a subject in need thereof, comprising:
(a) determining that the subject has a PI3Kα-associated cancer; and
(b) administering to the subject a combination therapy comprising Compound 1, or a pharmaceutically acceptable salt thereof, and a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

Some embodiments provide a method of treating cancer in a subject previously determined to have a PI3Kα-associated cancer, comprising administering to the subject
(a) Compound 1, or a pharmaceutically acceptable salt thereof, and
(b) one or more independently selected additional therapeutic agents selected from the group consisting of: a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, and a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

Some embodiments provide a method of treating cancer in a subject previously determined to have a PI3Kα-associated cancer, comprising administering to the subject of a combination therapy comprising Compound 1, or a pharmaceutically acceptable salt thereof, and a selective estrogen receptor modulator (SERM)/selective estrogen receptor degrader (SERD), a CDK4/6 inhibitor, a HER2 inhibitor, an EGFR inhibitor, an immune checkpoint inhibitor, a MEK inhibitor, a RAS inhibitor, a RAF inhibitor, a PIM (e.g., PIM1 and PIM3) inhibitor, or a combination of any of the foregoing. In some embodiments, Compound 1, or a pharmaceutically acceptable salt thereof, is administered with one additional therapeutic agent. In some embodiments, Compound 1, or a pharmaceutically acceptable salt thereof, is administered with two independently selected additional therapeutic agents.

In some embodiments, the one or more PI3Kα protein substitution/insertion/deletion are selected from the group consisting of E542A, E542G, E542K, E542Q, E542V, E545A, E545D, E545G, E545K, E545Q, M1043I, M1043L, M1043T, M1043V, H1047L, H1047Q, H1047R, H1047Y, G1049R, and combinations thereof. In some embodiments, the PI3Kα protein substitution/insertion/deletion is H1047X, where X is any amino acid. In some embodiments, the PI3Kα protein substitution/insertion/deletion is H1047R. In some embodiments, the one or more PI3Kα protein substitution/insertion/deletions are selected from the group consisting of E542A, E542G, E542K, E542Q, E542V, E545A, E545D, E545G, E545K, E545Q, and H1047R.

In some embodiments, the cancer (e.g., PI3Kα-associated cancer) is selected from a hematological cancer and a solid tumor.

In some embodiments, the cancer was refractory to one or more prior therapies. In some embodiments, the subject experienced dose-limiting toxicity to one or more prior therapies. In some embodiments, the one or more prior therapies comprises a PI3K inhibitor.

In some embodiments, the cancer is unresectable and/or metastatic.

In some embodiments, the cancer is locally advanced. In some embodiments, the cancer is unresectable. In some embodiments, the cancer is metastatic. In some embodiments, the cancer is metastatic brain cancer, as described herein. In some embodiments, the cancer is metastatic spinal cord cancer, as described herein.

In some embodiments, the cancer is selected from breast cancer (including both HER2⁺ and HER2⁻ breast cancer, ER⁺ breast cancer, and triple negative breast cancer), endometrial cancer, lung cancer (including adenocarcinoma lung cancer and squamous cell lung carcinoma), esophageal cancer (including esophageal squamous cell carcinoma), ovarian cancer, colorectal cancer, esophagastric adenocarcinoma, gastric cancer, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), prostate cancer, kidney cancer, pancreatic neuroendocine neoplasms (pNETs), stomach cancer, esophageal cancer, acute myeloid leukemia, relapsed and refractory multiple myeloma, and pancreatic cancer.

In some embodiments, the cancer is selected from breast cancer (including both HER2⁺ and HER2⁻ breast cancer, ER⁺ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pancreatic neuroendocine neoplasms (pNETs), stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments, the cancer is a gynecologic cancer. In some embodiments, the gynecologic cancer is endometrial cancer, ovarian cancer, or cervical cancer.

In some embodiments, the cancer is endometrial cancer that is not deficient in DNA mismatch repair (dMMR), i.e., the endometrial cancer is not dMMR.

In some embodiments, the cancer is selected from breast cancer, lung cancer, endometrial cancer, esophageal cancer, gastric cancer, ovarian cancer, colorectal cancer, bladder cancer, head and neck cancer, thyroid cancer, prostate cancer, glioma, and cervical cancer.

In some embodiments, the cancer is breast cancer.

In some embodiments, the cancer is lung cancer.

In some embodiments, the cancer is endometrial cancer.

In some embodiments, the cancer is esophageal cancer.

In some embodiments, the cancer is gastric cancer.

In some embodiments, the cancer is ovarian cancer.

In some embodiments, the cancer is colorectal cancer.

In some embodiments, the cancer is bladder cancer.

In some embodiments, the cancer is head and neck cancer.

In some embodiments, the cancer is thyroid cancer.

In some embodiments, the cancer is prostate cancer.

In some embodiments, the cancer is glioma.

In some embodiments, the cancer is cervical cancer.

In some embodiments, the cancer described herein is a HER2⁺ cancer. In some embodiments, the cancer described herein is a HER2⁻ cancer.

In some embodiments, the cancer described herein is a HER2-low cancer. In some embodiments, the cancer has a HER2 score (e.g., an IHC score) of 0, +1, or +2. In some embodiments, the cancer has a HER2 score (e.g., an IHC score) of 0 or +1. In some embodiments, the cancer has a HER2 score of 0. In some embodiments, the cancer has a HER2 score of +1. In some embodiments, the cancer has a HER2 score of +2. In some embodiments, the cancer has a HER2 score of +2 or +3. In some embodiments, the cancer has a HER2 score of +3.

In some embodiments, the cancer described herein is a hormone receptor positive (HR⁺) cancer. In some embodiments, the cancer described herein is a HER2⁻ and HR⁺ cancer. In some embodiments, the cancer described herein is ER⁺. In some embodiments, the cancer described herein is PR⁺. In some embodiments, the cancer is HR⁺/ER⁻ breast cancer.

In some embodiments, the cancer described herein is HR+ and has a HER2 score of 0 or +1. In some embodiments, the cancer described herein is HR+ and has a HER2 score of 0. In some embodiments, the cancer described herein is HR+ and has a HER2 score+1.

In some embodiments, the cancer described herein is HR+ and has a HER2 score of +2 or +3. In some embodiments, the cancer described herein is HR+ and has a HER2 score of +2. In some embodiments, the cancer described herein is HR+ and has a HER2 score+3.

In some embodiments the subject has been previously identified or determined not to have an activating mutation in AKT and/or PTEN.

In some embodiments, the PI3Kα-associated cancer is selected from the cancers described in Table 1 and Table 2.

TABLE 1
PI3Kα Protein Amino Acid Substitutions/Insertions/Deletions^{A}

| Amino | Non-Limiting | |
|---|---|---|
| Acid | Exemplary | Non-Limiting Exemplary PI3Kα |
| Position | Mutations | Associated Cancer(s) |
| 1 | M1 (Translation | Astrocytoma |
| | Start Site) | Glioblastoma Multiforme |
| 4 | R4* (Nonsense | Glioblastoma Multiforme |
| | Mutation) | |
| 9 | E9G | Stomach Adenocarcinoma |
| 10 | L10_M16del | Glioblastoma Multiforme |
| 11 | W11L, W11S, | Lung Adenocarcinoma, |
| | W11_P18del (In | Oligodendroglioma, |
| | Frame Deletion) | Uterine Endometrioid Carcinoma |
| 12 | G12D | Uterine Endometrioid Carcinoma |
| 13 | I13T | Colon Adenocarcinoma |
| 19 | R19I | Uterine Endometrioid Carcinoma |
| 27 | P27T | Hepatocellular Carcinoma |
| 36 | C36Y | Uterine Endometrioid Carcinoma |
| 38 | R38C, | Uterine Endometrioid Carcinoma |
| | R38H, | Papillary Renal Cell Carcinoma |
| | R38L, | Papillary Stomach Adenocarcinoma |
| | R38S | Mucinous Adenocarcinoma of the Colon and Rectum |
| | | Glioblastoma Multiforme |
| | | Cervical Squamous Cell Carcinoma |
| | | Hepatocellular Carcinoma |
| | | Uterine Endometrioid Carcinoma |
| | | Diffuse Type Stomach Adenocarcinoma |
| | | Lung Squamous Cell Carcinoma |
| | | Uterine Endometrioid Carcinoma |
| 39 | E39G, | Uterine Endometrioid Carcinoma |
| | E39K | Glioblastoma Multiforme |
| 57 | P57L | Cutaneous Melanoma |
| 65 | E65K | Lung Squamous Cell Carcinoma |
| 66 | S66C | Bladder Urothelial Carcinoma |
| 69 | I69N | Colon Adenocarcinoma |
| 71 | V71I | Head and Neck Squamous Cell Carcinoma |
| 75 | Q75E | Bladder Urothelial Carcinoma |
| | | Cervical Squamous Cell Carcinoma |
| | | Head and Neck Squamous Cell Carcinoma |
| 78 | E78* (nonsense | Lung Squamous Cell Carcinoma |
| | mutation) | |
| 80 | E80K | Uterine Mixed Endometrial Carcinoma |
| 81 | E81* (nonsense | Colon Adenocarcinoma |
| | mutation), E81del | Glioblastoma Multiforme |
| | (in frame | Colon Adenocarcinoma |
| | deletion), E81K | Uterine Serous Carcinoma/Uterine Papillary Serous |
| | | Carcinoma |
| | | Glioblastoma Multiforme |
| | | Uterine Endometrioid Carcinoma |
| | | Lung Squamous Cell Carcinoma |
| | | Mucinous Adenocarcinoma of the Colon and Rectum |
| | | Breast Invasive Ductal Carcinoma |
| | | Cervical Squamous Cell Carcinoma |
| | | Head and Neck Squamous Cell Carcinoma |
| 83 | F83L, F83S | Breast Invasive Lobular Carcinoma |
| 84 | D84H | Lung Adenocarcinoma |
| 86 | T86S | Hepatocellular Carcinoma |
| 87 | R87T | Lung Adenocarcinoma |
| 88 | R88Q | Breast Invasive Ductal Carcinoma |
| | | Rectal Adenocarcinoma |
| | | Colon Adenocarcinoma |
| | | Prostate Adenocarcinoma |
| | | Cervical Squamous Cell Carcinoma |
| | | Uterine Carcinosarcoma/Uterine Malignant Mixed |
| | | Mullerian Tumor |
| | | Tubular Stomach Adenocarcinoma |
| | | Oligodendroglioma |
| | | Mucinous Stomach Adenocarcinoma |
| | | Glioblastoma Multiforme |
| | | Stomach Adenocarcinoma |
| | | Uterine Endometrioid Carcinoma |
| | | Uterine Mixed Endometrial Carcinoma |
| | | Head and Neck Squamous Cell Carcinoma |
| | | Mucinous Adenocarcinoma of the Colon and Rectum |
| | | Breast Invasive Lobular Carcinoma |
| | | Intestinal Type Stomach Adenocarcinoma |
| | | Bladder Urothelial Carcinoma |
| 90 | C90G, C90R, | Glioblastoma Multiforme |
| | C90Y | Cervical Squamous Cell Carcinoma |
| 93 | R93P, R93Q, | Mucinous Adenocarcinoma of the Colon and Rectum |
| | R93W | Stomach Adenocarcinoma |
| | | Glioblastoma Multiforme |
| | | Uterine Endometrioid Carcinoma |
| | | Tubular Stomach Adenocarcinoma |
| | | Mucinous Stomach Adenocarcinoma |
| | | Bladder Urothelial Carcinoma |
| | | Cervical Squamous Cell Carcinoma |
| | | Colon Adenocarcinoma |
| 102 | I102del | Uterine Endometrioid Carcinoma |
| 103 | E103G, | Glioblastoma Multiforme |
| | E103_G106delins | Breast Invasive Ductal Carcinoma |
| | D (In Frame | |
| | Deletion), | |
| | E103_P104del (In | |
| | Frame Deletion) | |
| 104 | P104L, P104R, | Breast Invasive Ductal Carcinoma |
| | P104T | Head and Neck Squamous Cell Carcinoma |
| | | Lung Adenocarcinoma |
| | | Colon Adenocarcinoma |
| | | Uterine Serous Carcinoma/Uterine Papillary Serous |
| | | Carcinoma |
| 105 | V105del, | Uterine Endometrioid Carcinoma |
| | V105_R108del | Breast Invasive Ductal Carcinoma |
| 106 | G106D, G106R, | Uterine Mixed Endometrial Carcinoma |
| | G106S, G106V, | Breast Invasive Ductal Carcinoma |
| | G106_R108del | Mucinous Adenocarcinoma of the Colon and Rectum |
| | (In Frame | Mucinous Carcinoma |
| | Deletion), | Oligodendroglioma |
| | G106_N107del | Uterine Carcinosarcoma/Uterine Malignant Mixed |
| | (In Frame | Mullerian Tumor |
| | Deletion) | Uterine Serous Carcinoma/Uterine Papillary Serous |
| | | Carcinoma |
| | | Uterine Endometrioid Carcinoma |
| | | Rectal Adenocarcinoma |
| | | Lung Squamous Cell Carcinoma |
| | | Cervical Squamous Cell Carcinoma |
| | | Tubular Stomach Adenocarcinoma |
| | | Uterine Endometrioid Carcinoma |
| 107 | N107S | Uterine Endometrioid Carcinoma |
| | | Lung Adenocarcinoma |
| 108 | R108C, R108H, | Prostate Adenocarcinoma |
| | R108L | Uterine Endometrioid Carcinoma |
| | | Glioblastoma Multiforme |
| | | Uterine Carcinosarcoma/Uterine Malignant Mixed |
| | | Mullerian Tumor |
| | | Mucinous Adenocarcinoma of the Colon and Rectum |
| | | Tubular Stomach Adenocarcinoma |
| | | Colon Adenocarcinoma |
| | | Uterine Serous Carcinoma/Uterine Papillary Serous |
| | | Carcinoma |
| | | Uterine Mixed Endometrial Carcinoma |
| | | Breast Invasive Ductal Carcinoma |
| | | Lung Squamous Cell Carcinoma |
| 109 | E109_I112delins | Breast Invasive Ductal Carcinoma |
| | D (In Frame | |
| | Deletion) | |
| 110 | E110del | Uterine Endometrioid Carcinoma |
| | | Oligodendroglioma |
| | | Breast Invasive Lobular Carcinoma |
| | | Breast Invasive Ductal Carcinoma |
| | | Uterine Mixed Endometrial Carcinoma |
| | | Colon Adenocarcinoma |
| | | Head and Neck Squamous Cell Carcinoma |
| | | Lung Adenocarcinoma |
| | | Papillary Thyroid Cancer |
| 111 | K111del, K111E, | Uterine Endometrioid Carcinoma |
| | K111N, K111R, | Breast Invasive Ductal Carcinoma |
| | K111_L113del | Oligodendroglioma |
| | (In Frame | Head and Neck Squamous Cell Carcinoma |
| | Deletion) | Colon Adenocarcinoma |
| | | Intestinal Type Stomach Adenocarcinoma |
| | | Stomach Adenocarcinoma |
| | | Uterine Endometrioid Carcinoma |
| | | Lung Adenocarcinoma |
| | | Esophageal Adenocarcinoma |
| | | Lung Squamous Cell Carcinoma |
| | | Glioblastoma Multiforme |
| 113 | L113del | Uterine Endometrioid Carcinoma |
| 115 | R115L, R115P | Serous Ovarian Cancer |
| | | Bladder Urothelial Carcinoma |
| | | Uterine Carcinosarcoma/Uterine Malignant Mixed |
| | | Mullerian Tumor |
| | | Uterine Endometrioid Carcinoma |
| | | Uterine Serous Carcinoma/Uterine Papillary Serous |
| | | Carcinoma |
| | | Rectal Adenocarcinoma |
| | | Cervical Squamous Cell Carcinoma |
| 116 | E116K | Rectal Adenocarcinoma |
| 118 | G118D | Glioblastoma Multiforme |
| | | Breast Invasive Ductal Carcinoma |
| | | Uterine Endometrioid Carcinoma |
| | | Bladder Urothelial Carcinoma |
| | | Oligodendroglioma |
| | | Esophageal Adenocarcinoma |
| | | Astrocytoma |
| | | Lung Squamous Cell Carcinoma |
| | | Breast Invasive Lobular Carcinoma |
| | | Uterine Serous Carcinoma/Uterine Papillary Serous |
| | | Carcinoma |
| | | Mucinous Carcinoma |
| | | Uterine Mixed Endometrial Carcinoma |
| | | Stomach Adenocarcinoma |
| | | Pancreatic Adenocarcinoma |
| | | Papillary Thyroid Cancer |
| | | Rectal Adenocarcinoma |
| 123 | M123I | Lung Adenocarcinoma |
| 124 | P124A | Lung Adenocarcinoma |
| 151 | V151M | Bladder Urothelial Carcinoma |
| | | Astrocytoma |
| 165 | Y165H | Uterine Mixed Endometrial Carcinoma |
| 170 | N170S | Uterine Endometrioid Carcinoma |
| 182 | Y182H | Stomach Adenocarcinoma |
| 213 | H213N | Cutaneous Melanoma |
| 224 | A224S | Colon Adenocarcinoma |
| 239 | L239R | Colon Adenocarcinoma |
| 258 | D258N | Rectal Adenocarcinoma |
| 262 | L262I | Cutaneous Melanoma |
| 266 | P266T | Uterine Endometrioid Carcinoma |
| 267 | L267M | Cutaneous Melanoma |
| 272 | Y272* (Nonsense | Renal Clear Cell Carcinoma |
| | Mutation) | |
| 274 | R274K | Bladder Urothelial Carcinoma |
| 279 | L279I | Uterine Endometrioid Carcinoma |
| 282 | M282V | Uterine Endometrioid Carcinoma |
| 292 | S292I | Glioblastoma Multiforme |
| 296 | Q296E | Lung Adenocarcinoma |
| 300 | D300V | Lung Squamous Cell Carcinoma |
| 310 | R310C | Uterine Endometrioid Carcinoma |
| 322 | T322A | Uterine Endometrioid Carcinoma |
| 335 | R335G | Head and Neck Squamous Cell Carcinoma |
| 337 | K337N | Rectal Adenocarcinoma |
| 339 | L339I | Cervical Squamous Cell Carcinoma |
| | | Uterine Endometrioid Carcinoma |
| 342 | T342S | Lung Adenocarcinoma |
| 344 | V344A, V344G, | Uterine Endometrioid Carcinoma |
| | V344M | Mucinous Adenocarcinoma of the Colon and Rectum |
| | | Colon Adenocarcinoma |
| | | Cervical Squamous Cell Carcinoma |
| | | Rectal Adenocarcinoma |
| | | Head and Neck Squamous Cell Carcinoma |
| | | Breast Invasive Carcinoma (NOS) |
| | | Uterine Mixed Endometrial Carcinoma |
| | | Glioblastoma Multiforme |
| 345 | N345H, N345I, | Breast Invasive Lobular Carcinoma |
| | N345K, N345T, | Uterine Carcinosarcoma/Uterine Malignant Mixed |
| | N345Y | Mullerian Tumor |
| | | Uterine Serous Carcinoma/Uterine Papillary Serous |
| | | Carcinoma |
| | | Uterine Endometrioid Carcinoma |
| | | Breast Invasive Carcinoma (NOS) |
| | | Lung Adenocarcinoma |
| | | Mucinous Adenocarcinoma of the Colon and Rectum |
| | | Bladder Urothelial Carcinoma |
| | | Colon Adenocarcinoma |
| | | Leiomyosarcoma |
| | | Glioblastoma Multiforme |
| | | Uterine Endometrioid Carcinoma |
| | | Seminoma |
| | | Tubular Stomach Adenocarcinoma |
| | | Breast Invasive Ductal Carcinoma |
| | | Head and Neck Squamous Cell Carcinoma |
| | | Stomach Adenocarcinoma |
| | | Diffuse Type Stomach Adenocarcinoma |
| | | Prostate Adenocarcinoma |
| | | Breast Invasive Ductal Carcinoma |
| | | Oligodendroglioma |
| 350 | D350G, D350N | Lung Squamous Cell Carcinoma |
| | | Breast Invasive Ductal Carcinoma |
| | | Uterine Endometrioid Carcinoma |
| | | Colon Adenocarcinoma |
| | | Uterine Endometrioid Carcinoma |
| | | Mucinous Stomach Adenocarcinoma |
| | | Lung Adenocarcinoma |
| | | Breast Invasive Lobular Carcinoma |
| 351 | I351T | Uterine Endometrioid Carcinoma |
| 357 | R357Q | Uterine Endometrioid Carcinoma |
| | | Mucinous Adenocarcinoma of the Colon and Rectum |
| | | Colon Adenocarcinoma |
| 359 | G359R | Uterine Endometrioid Carcinoma |
| 363 | G363A | Head and Neck Squamous Cell Carcinoma |
| 364 | G364R | Uterine Mixed Endometrial Carcinoma |
| | | Intestinal Type Stomach Adenocarcinoma |
| | | Colon Adenocarcinoma |
| 365 | E365K, E365V | Uterine Endometrioid Carcinoma |
| | | Bladder Urothelial Carcinoma |
| | | Mucinous Adenocarcinoma of the Colon and Rectum |
| | | Diffuse Type Stomach Adenocarcinoma |
| | | Breast Invasive Ductal Carcinoma |
| | | Head and Neck Squamous Cell Carcinoma |
| 366 | P366R | Breast Invasive Ductal Carcinoma |
| 378 | C378F, C378R, | Uterine Serous Carcinoma/Uterine Papillary Serous |
| | C378Y | Carcinoma |
| | | Uterine Endometrioid Carcinoma |
| | | Oligodendroglioma |
| 379 | S379T | Cutaneous Melanoma |
| 380 | N380S | Diffuse Type Stomach Adenocarcinoma |
| 390 | D390N | Lung Adenocarcinoma |
| 392 | Y392H | Uterine Endometrioid Carcinoma |
| 398 | R398H | Breast Invasive Ductal Carcinoma |
| 399 | A399T | Cervical Squamous Cell Carcinoma |
| 401 | R401Q | Uterine Endometrioid Carcinoma |
| 405 | S405F | Intestinal Type Stomach Adenocarcinoma |
| 406 | I406V | Uterine Mixed Endometrial Carcinoma |
| 412 | R412Q | Stomach Adenocarcinoma |
| 417 | E417K | Bladder Urothelial Carcinoma |
| 418 | E418K | Uterine Endometrioid Carcinoma |
| | | Rectal Adenocarcinoma |
| | | Mucinous Carcinoma |
| | | Head and Neck Squamous Cell Carcinoma |
| | | Bladder Urothelial Carcinoma |
| 420 | C420R | Uterine Endometrioid Carcinoma |
| | | Tubular Stomach Adenocarcinoma |
| | | Lung Squamous Cell Carcinoma |
| | | Breast Invasive Ductal Carcinoma |
| | | Uterine Serous Carcinoma/Uterine Papillary Serous |
| | | Carcinoma |
| | | Colon Adenocarcinoma |
| | | Intestinal Type Stomach Adenocarcinoma |
| | | Stomach Adenocarcinoma |
| | | Head and Neck Squamous Cell Carcinoma |
| | | Breast Invasive Carcinoma (NOS) |
| | | Astrocytoma |
| | | Cervical Squamous Cell Carcinoma |
| 432 | Y432C | Cervical Squamous Cell Carcinoma |
| 447 | P447_L455del (In | Breast Invasive Ductal Carcinoma |
| | frame Deletion) | |
| 449 | P449L, P449S | Uterine Endometrioid Carcinoma |
| 450 | H450_P458del | Breast Invasive Ductal Carcinoma |
| | (In Frame | |
| | Deletion) | |
| 451 | G451R, G451V, | Head and Neck Squamous Cell Carcinoma |
| | G451_D454del | Bladder Urothelial Carcinoma |
| | (In Frame | Colon Adenocarcinoma |
| | Deletion) | |
| 452 | L452_G460del | Breast Invasive Ductal Carcinoma |
| | (In Frame | |
| | Deletion) | |
| 453 | E453del, E453K, | Oligodendroglioma |
| | E453Q, | Uterine Mixed Endometrial Carcinoma |
| | E453_G460delins | Intestinal Type Stomach Adenocarcinoma |
| | DDF (in Frame | Breast Invasive Ductal Carcinoma |
| | Deletion), | Breast Invasive Lobular Carcinoma |
| | E453_L455del | Head and Neck Squamous Cell Carcinoma |
| | | Astrocytoma |
| | | Stomach Adenocarcinoma |
| | | Bladder Urothelial Carcinoma |
| | | Lung Squamous Cell Carcinoma |
| | | Cervical Squamous Cell Carcinoma |
| | | Lung Adenocarcinoma |
| | | Mucinous Carcinoma |
| | | Uterine Endometrioid Carcinoma |
| | | Uterine Serous Carcinoma/Uterine Papillary Serous |
| | | Carcinoma |
| | | Glioblastoma Multiforme |
| | | Colon Adenocarcinoma |
| 454 | D454Y | Uterine Endometrioid Carcinoma |
| 455 | L455_G463del | Glioblastoma Multiforme |
| | (In Frame | |
| | Deletion) | |
| 463 | G463_N465delins | Uterine Endometrioid Carcinoma |
| | D (In Frame | |
| | Deletion) | |
| 469 | E469A, | Rectal Adenocarcinoma |
| | E469delinsDK | Breast Invasive Ductal Carcinoma |
| | (In Frame | |
| | Insertion) | |
| 471 | P471A, P471L | Bladder Urothelial Carcinoma |
| | | Rectal Adenocarcinoma |
| | | Uterine Endometrioid Carcinoma |
| | | Oligoastrocytoma |
| | | Hepatocellular Carcinoma |
| 474 | E474A | Prostate Adenocarcinoma |
| 475 | L475F | Cutaneous Melanoma |
| 479 | W479* | Uterine Endometrioid Carcinoma |
| 495 | H495L, H495Y | Lung Squamous Cell Carcinoma |
| | | Uterine Endometrioid Carcinoma |
| 499 | S499F | Bladder Urothelial Carcinoma |
| 519 | R519G | Head and Neck Squamous Cell Carcinoma |
| 520 | D520V | Breast Invasive Lobular Carcinoma |
| 522 | E522A | Uterine Endometrioid Carcinoma |
| 531 | L531V | Breast Invasive Ductal Carcinoma |
| 539 | P539R, P539S | Breast Invasive Ductal Carcinoma |
| | | Pancreatic Adenocarcinoma |
| | | Uterine Endometrioid Carcinoma |
| | | Lung Squamous Cell Carcinoma |
| 542 | E542A, E542G, | Uterine Serous Carcinoma/Uterine Papillary Serous |
| | E542K, E542Q, | Carcinoma |
| | E542V | Uterine Endometrioid Carcinoma |
| | | Colon Adenocarcinoma |
| | | Prostate Adenocarcinoma |
| | | Breast Invasive Ductal Carcinoma |
| | | Breast Invasive Lobular Carcinoma |
| | | Endocervical Adenocarcinoma |
| | | Intestinal Type Stomach Adenocarcinoma |
| | | Prostate Adenocarcinoma |
| | | Papillary Renal Cell Carcinoma |
| | | Oligoastrocytoma |
| | | Hepatocellular Carcinoma |
| | | Bladder Urothelial Carcinoma |
| | | Mucinous Adenocarcinoma of the Colon and Rectum |
| | | Diffuse Type Stomach Adenocarcinoma |
| | | Lung Squamous Cell Carcinoma |
| | | Signet Ring Cell Carcinoma of the Stomach |
| | | Head and Neck Squamous Cell Carcinoma |
| | | Breast Invasive Carcinoma (NOS) |
| | | Mucinous Carcinoma |
| | | Breast Invasive Ductal Carcinoma |
| | | Cervical Squamous Cell Carcinoma |
| | | Glioblastoma Multiforme |
| | | Lung Adenocarcinoma |
| 545 | E545A, E545D, | Uterine Serous Carcinoma/Uterine Papillary Serous |
| | E545G, E545K, | Carcinoma |
| | E545Q | Uterine Endometrioid Carcinoma |
| | | Colon Adenocarcinoma |
| | | Prostate Adenocarcinoma |
| | | Breast Invasive Ductal Carcinoma |
| | | Breast Invasive Lobular Carcinoma |
| | | Endocervical Adenocarcinoma |
| | | Intestinal Type Stomach Adenocarcinoma |
| | | Papillary Renal Cell Carcinoma |
| | | Oligoastrocytoma |
| | | Hepatocellular Carcinoma |
| | | Bladder Urothelial Carcinoma |
| | | Mucinous Adenocarcinoma of the Colon and Rectum |
| | | Diffuse Type Stomach Adenocarcinoma |
| | | Lung Squamous Cell Carcinoma |
| | | Signet Ring Cell Carcinoma of the Stomach |
| | | Head and Neck Squamous Cell Carcinoma |
| | | Breast Invasive Carcinoma (NOS) |
| | | Mucinous Carcinoma |
| | | Cervical Squamous Cell Carcinoma |
| | | Glioblastoma Multiforme |
| | | Oligodendroglioma |
| | | Lung Adenocarcinoma |
| | | Serous Ovarian Cancer |
| | | Uterine Carcinosarcoma/Uterine Malignant Mixed |
| | | Mullerian Tumor |
| | | Astrocytoma |
| | | Rectal Adenocarcinoma |
| | | Stomach Adenocarcinoma |
| | | Cutaneous Melanoma |
| | | Esophageal Squamous Cell Carcinoma |
| | | Breast Invasive Mixed Mucinous Carcinoma |
| | | Intrahepatic Cholangiocarcinoma |
| | | Renal Clear Cell Carcinoma |
| | | Seminoma |
| | | Esophageal Adenocarcinoma |
| | | Tubular Stomach Adenocarcinoma |
| | | Uterine Mixed Endometrial Carcinoma |
| 546 | Q546E, Q546H, | Uterine Endometrioid Carcinoma |
| | Q546K, Q546P, | Rectal Adenocarcinoma |
| | Q546R | Oligodendroglioma |
| | | Stomach Adenocarcinoma |
| | | Esophageal Adenocarcinoma |
| | | Bladder Urothelial Carcinoma |
| | | Breast Invasive Carcinoma (NOS) |
| | | Breast Invasive Ductal Carcinoma |
| | | Colon Adenocarcinoma |
| | | Glioblastoma Multiforme |
| | | Uterine Serous Carcinoma/Uterine Papillary Serous |
| | | Carcinoma |
| | | Undifferentiated Pleomorphic Sarcoma/Malignant Fibrous |
| | | Histiocytoma/High-Grade Spindle Cell Sarcoma |
| | | Astrocytoma |
| | | Uterine Carcinosarcoma/Uterine Malignant Mixed |
| | | Mullerian Tumor |
| | | Oligoastrocytoma |
| | | Breast Invasive Lobular Carcinoma |
| | | Tubular Stomach Adenocarcinoma |
| | | Head and Neck Squamous Cell Carcinoma |
| | | Cervical Squamous Cell Carcinoma |
| | | Intestinal Type Stomach Adenocarcinoma |
| 547 | E547D, E547K | Lung Squamous Cell Carcinoma |
| | | Stomach Adenocarcinoma |
| 552 | W552C | Bladder Urothelial Carcinoma |
| 569 | L569I | Uterine Serous Carcinoma/Uterine Papillary Serous |
| | | Carcinoma |
| | | Prostate Adenocarcinoma |
| 576 | S576Y | Uterine Endometrioid Carcinoma |
| 581 | A581S | Cutaneous Melanoma |
| 589 | D589N | Cervical Squamous Cell Carcinoma |
| 600 | E600K, E600V | Uterine Endometrioid Carcinoma |
| | | Bladder Urothelial Carcinoma |
| | | Lung Adenocarcinoma |
| | | Breast Invasive Lobular Carcinoma |
| | | Papillary Stomach Adenocarcinoma |
| 603 | D603H | Breast Invasive Ductal Carcinoma |
| 604 | C604R | Uterine Endometrioid Carcinoma |
| | | Uterine Carcinosarcoma/Uterine Malignant Mixed |
| | | Mullerian Tumor |
| | | Uterine Serous Carcinoma/Uterine Papillary Serous |
| | | Carcinoma |
| | | Head and Neck Squamous Cell Carcinoma |
| 606 | Y606C | Head and Neck Squamous Cell Carcinoma |
| 607 | P607Q | Cutaneous Melanoma |
| 609 | P609H | Colon Adenocarcinoma |
| 614 | F614I | Breast Invasive Ductal Carcinoma |
| 617 | R617Q, R617W | Uterine Endometrioid Carcinoma |
| 617 | R617W | Uterine Endometrioid Carcinoma |
| 629 | S629C | Breast Invasive Ductal Carcinoma |
| 636 | V636L | Bladder Urothelial Carcinoma |
| 642 | E642K | Uterine Serous Carcinoma/Uterine Papillary Serous |
| | | Carcinoma |
| 643 | Q643H | Uterine Endometrioid Carcinoma |
| 658 | L658F | Colon Adenocarcinoma |
| 667 | F667L | Uterine Endometrioid Carcinoma |
| | | Lung Squamous Cell Carcinoma |
| | | Uterine Serous Carcinoma/Uterine Papillary Serous |
| | | Carcinoma |
| 673 | S673T | Breast Invasive Ductal Carcinoma |
| 674 | E674* (Nonsense | Papillary Thyroid Cancer |
| | mutation), | Cutaneous Melanoma |
| | E674D, E674Q | Bladder Urothelial Carcinoma |
| 682 | Q682K, | Cutaneous Melanoma |
| | Q682Rfs*18 | Glioblastoma Multiforme |
| | (Frame Shift | |
| | Deletion) | |
| 683 | R683M | Cutaneous Melanoma |
| 684 | F684L | Uterine Endometrioid Carcinoma |
| 693 | R693H | Cervical Squamous Cell Carcinoma |
| 710 | E710Q | Bladder Urothelial Carcinoma |
| 711 | K711N | Astrocytoma |
| 722 | E722K | Colon Adenocarcinoma |
| 725 | D725G, D725N | Colon Adenocarcinoma |
| | | Uterine Endometrioid Carcinoma |
| 726 | E726K | Cervical Squamous Cell Carcinoma |
| | | Uterine Endometrioid Carcinoma |
| | | Breast Invasive Ductal Carcinoma |
| | | Hepatocellular Carcinoma |
| | | Lung Adenocarcinoma |
| | | Esophageal Squamous Cell Carcinoma |
| | | Esophageal Adenocarcinoma |
| | | Rectal Adenocarcinoma |
| | | Head and Neck Squamous Cell Carcinoma |
| | | Lung Squamous Cell Carcinoma |
| | | Breast Invasive Lobular Carcinoma |
| | | Bladder Urothelial Carcinoma |
| | | Colon Adenocarcinoma |
| 729 | K729N | Cutaneous Melanoma |
| 732 | M732I | Colon Adenocarcinoma |
| 737 | E737K | Cutaneous Melanoma |
| 741 | R741Q | Serous Ovarian Cancer |
| 744 | F744I | Stomach Adenocarcinoma |
| 746 | D746Y | Cutaneous Melanoma |
| 749 | Q749H | Cutaneous Melanoma |
| 752 | L752V | Bladder Urothelial Carcinoma |
| 766 | L766F | Breast Invasive Ductal Carcinoma |
| 770 | R770Q | Uterine Endometrioid Carcinoma |
| 773 | S773F | Cutaneous Melanoma |
| 777 | R777M, R777K | Cutaneous Melanoma |
| | | Colon Adenocarcinoma |
| 791 | E791Q | Bladder Urothelial Carcinoma |
| 811 | M811I | Uterine Endometrioid Carcinoma |
| 816 | I816S | Uterine Endometrioid Carcinoma |
| 818 | R818C, R818H | Cutaneous Melanoma |
| | | Uterine Endometrioid Carcinoma |
| 849 | E849K | Serous Ovarian Cancer |
| 852 | R852Q | Leiomyosarcoma |
| | | Uterine Serous Carcinoma/Uterine Papillary Serous |
| | | Carcinoma |
| 865 | G865D | Cutaneous Melanoma |
| 866 | L866F, L866W | Cervical Squamous Cell Carcinoma |
| | | Uterine Endometrioid Carcinoma |
| 879 | Q879R | Stomach Adenocarcinoma |
| 886 | K886E | Undifferentiated Pleomorphic Sarcoma/Malignant Fibrous |
| | | Histiocytoma/High-Grade Spindle Cell Sarcoma |
| 901 | C901F | Uterine Endometrioid Carcinoma |
| | | Astrocytoma |
| | | Breast Invasive Ductal Carcinoma |
| | | Head and Neck Squamous Cell Carcinoma |
| 903 | G903E | Uterine Serous Carcinoma/Uterine Papillary Serous |
| | | Carcinoma |
| 905 | C905S | Head and Neck Squamous Cell Carcinoma |
| 909 | F909C | Esophageal Adenocarcinoma |
| 914 | G914R | Uterine Serous Carcinoma/Uterine Papillary Serous |
| | | Carcinoma |
| | | Astrocytoma |
| 929 | L929M | Uterine Endometrioid Carcinoma |
| 930 | F930V | Uterine Endometrioid Carcinoma |
| 939 | D939G | Breast Invasive Carcinoma (NOS) |
| | | Uterine Endometrioid Carcinoma |
| | | Breast Invasive Ductal Carcinoma |
| 948 | K948E | Intestinal Type Stomach Adenocarcinoma |
| 951 | R951C | Rectal Adenocarcinoma |
| 953 | P953S | Uterine Endometrioid Carcinoma |
| 956 | L956F | Bladder Urothelial Carcinoma |
| 958 | Q958R | Uterine Mixed Endometrial Carcinoma |
| 970 | E970K | Esophageal Squamous Cell Carcinoma |
| | | Head and Neck Squamous Cell Carcinoma |
| | | Mucinous Adenocarcinoma of the Colon and Rectum |
| | | Colon Adenocarcinoma |
| 971 | C971R | Head and Neck Squamous Cell Carcinoma |
| 978 | E978K | Bladder Urothelial Carcinoma |
| 979 | R979G | Pancreatic Adenocarcinoma |
| 985 | Y985* | Pleural Mesothelioma, Biphasic Type |
| 989 | L989V | Breast Invasive Ductal Carcinoma |
| 992 | R992L, R992P | Bladder Urothelial Carcinoma |
| | | Uterine Serous Carcinoma/Uterine Papillary Serous |
| | | Carcinoma |
| | | Mucinous Carcinoma |
| 997 | L997I | Uterine Endometrioid Carcinoma |
| 1002 | F1002L | Uterine Endometrioid Carcinoma |
| 1004 | M1004I, | Uterine Endometrioid Carcinoma |
| | M1004R, | Breast Invasive Ductal Carcinoma |
| | M1004V | Bladder Urothelial Carcinoma |
| | | Lung Squamous Cell Carcinoma |
| 1005 | M1005V | Oligodendroglioma |
| 1006 | L1006R | Uterine Endometrioid Carcinoma |
| 1007 | G1007R | Uterine Endometrioid Carcinoma |
| | | Breast Invasive Ductal Carcinoma |
| | | Head and Neck Squamous Cell Carcinoma |
| | | Colon Adenocarcinoma |
| | | Endocervical Adenocarcinoma |
| 1012 | E1012Q | Bladder Urothelial Carcinoma |
| 1015 | S1015Y | Mucinous Adenocarcinoma of the Colon and Rectum |
| 1016 | F1016C | Uterine Endometrioid Carcinoma |
| 1017 | D1017N | Pancreatic Adenocarcinoma |
| 1020 | A1020T | Uterine Endometrioid Carcinoma |
| 1021 | Y1021C, Y1021H | Uterine Carcinosarcoma/Uterine Malignant Mixed |
| | | Mullerian Tumor |
| | | Colon Adenocarcinoma |
| | | Breast Invasive Ductal Carcinoma |
| | | Uterine Serous Carcinoma/Uterine Papillary Serous |
| | | Carcinoma |
| | | Uterine Endometrioid Carcinoma |
| | | Stomach Adenocarcinoma |
| | | Mucinous Adenocarcinoma of the Colon and Rectum |
| | | Tubular Stomach Adenocarcinoma |
| 1025 | T1025A, T1025S | Uterine Endometrioid Carcinoma |
| | | Breast Invasive Ductal Carcinoma |
| | | Mucinous Adenocarcinoma of the Colon and Rectum |
| | | Uterine Mixed Endometrial Carcinoma |
| 1023 | R1023Q^{†} | Colorectal Cancer |
| 1026 | L1026I | Cutaneous Melanoma |
| 1029 | D1029H | Uterine Serous Carcinoma/Uterine Papillary Serous |
| | | Carcinoma |
| 1037 | E1037K | Breast Invasive Ductal Carcinoma |
| 1040 | M1040I, M1040V | Head and Neck Squamous Cell Carcinoma |
| | | Breast Invasive Ductal Carcinoma |
| 1043 | M1043I, | Breast Invasive Lobular Carcinoma |
| | M1043L, | Tubular Stomach Adenocarcinoma |
| | M1043T, | Uterine Endometrioid Carcinoma |
| | M1043V | Mucinous Adenocarcinoma of the Colon and Rectum |
| | | Papillary Thyroid Cancer |
| | | Esophageal Squamous Cell Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine Papillary Serous |
| | | Carcinoma |
| | | Glioblastoma Multiforme |
| | | Head and Neck Squamous Cell Carcinoma |
| 1044 | N1044I, N1044K, | Uterine Endometrioid Carcinoma |
| | N1044Y | Breast Invasive Ductal Carcinoma |
| 1045 | D1045A, | Uterine Endometrioid Carcinoma |
| | D1045V | Lung Squamous Cell Carcinoma |
| 1047 | H1047L, | Esophageal Squamous Cell Carcinoma |
| | H1047Q, | Uterine Endometrioid Carcinoma |
| | H1047R, H1047Y | Hepatocellular Carcinoma |
| | | Cutaneous Melanoma |
| | | Mucinous Adenocarcinoma of the Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial Carcinoma |
| | | Breast Invasive Ductal Carcinoma |
| | | Renal Clear Cell Carcinoma |
| | | Uterine Serous Carcinoma/Uterine Papillary Serous |
| | | Carcinoma |
| | | Head and Neck Squamous Cell Carcinoma |
| | | Lung Squamous Cell Carcinoma |
| | | Breast Invasive Lobular Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach Adenocarcinoma |
| | | Diffuse Type Stomach Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic Sarcoma/Malignant Fibrous |
| | | Histiocytoma/High-Grade Spindle Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial Carcinoma |
| 1058 | I1058M | Uterine Carcinosarcoma/Uterine Malignant Mixed |
| | | Mullerian Tumor |
| 1065 | H1065L | Breast Invasive Lobular Carcinoma |
| 1066 | A1066V | Uterine Mixed Endometrial Carcinoma |
| 1068 | N1068Y, | Pleural Mesothelioma, Epithelioid Type |
| | N1068fs*5 | Dedifferentiated Liposarcoma |
| | (Frame Shift | Head and Neck Squamous Cell Carcinoma |
| | Insertion) | |
| 1069 | *1069Wext*4 | Glioblastoma Multiforme |
| | (nonstop | |
| | Mutation) | |

^{A}Unless noted otherwise, the mutations of Table 1 are found in cBioPortal database derived from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
^{†}Velho S, Oliveira C, Ferreira A, Ferreira AC, Suriano G, Schwartz S Jr, Duval A, Carneiro F, Machado J C, Hamelin R, Seruca R. The prevalence of PIK3CA mutations in gastric and colon cancer. Eur J Cancer. 2005 July; 41(11): 1649-54. doi: 10.1016/j.ejca.2005.04.022. PMID: 15994075.

TABLE 2
Additional PI3Kα Protein Amino Acid Substitutions/Insertions/Deletions^{A}

| | Non-Limiting | |
|---|---|---|
| Amino Acid | Exemplary | |
| Position | Mutations | Non-Limiting Exemplary PI3Kα Associated Cancer(s) |
| 1043 | M1043I, | Breast Invasive Lobular Carcinoma |
| | M1043L, | Tubular Stomach Adenocarcinoma |
| | M1043T, | Uterine Endometrioid Carcinoma |
| | M1043V | Mucinous Adenocarcinoma of the Colon and Rectum |
| | | Papillary Thyroid Cancer |
| | | Esophageal Squamous Cell Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine Papillary Serous |
| | | Carcinoma |
| | | Glioblastoma Multiforme |
| | | Head and Neck Squamous Cell Carcinoma |
| 1044 | N1044I, N1044K, | Uterine Endometrioid Carcinoma |
| | N1044Y | Breast Invasive Ductal Carcinoma |
| 1045 | D1045A, | Uterine Endometrioid Carcinoma |
| | D1045V | Lung Squamous Cell Carcinoma |
| 1047 | H1047L, | Esophageal Squamous Cell Carcinoma |
| | H1047Q, | Uterine Endometrioid Carcinoma |
| | H1047R, H1047Y | Hepatocellular Carcinoma |
| | | Cutaneous Melanoma |
| | | Mucinous Adenocarcinoma of the Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial Carcinoma |
| | | Breast Invasive Ductal Carcinoma |
| | | Renal Clear Cell Carcinoma |
| | | Uterine Serous Carcinoma/Uterine Papillary Serous |
| | | Carcinoma |
| | | Head and Neck Squamous Cell Carcinoma |
| | | Lung Squamous Cell Carcinoma |
| | | Breast Invasive Lobular Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach Adenocarcinoma |
| | | Diffuse Type Stomach Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic Sarcoma/Malignant Fibrous |
| | | Histiocytoma/High-Grade Spindle Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial Carcinoma |
| 1058 | I1058M | Uterine Carcinosarcoma/Uterine Malignant Mixed |
| | | Mullerian Tumor |
| 1065 | H1065L | Breast Invasive Lobular Carcinoma |
| 1066 | A1066V | Uterine Mixed Endometrial Carcinoma |
| 1068 | N1068Y, | Pleural Mesothelioma, Epithelioid Type |
| | N1068fs*5 | Dedifferentiated Liposarcoma |
| | (Frame Shift | Head and Neck Squamous Cell Carcinoma |
| | Insertion) | |

^{A}Unless noted otherwise, the mutations of Table 2 are found in cBioPortal database derived from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
† Velho S, Oliveira C, Ferreira A, Ferreira AC, Suriano G, Schwartz S Jr, Duval A, Carneiro F, Machado JC, Hamelin R, Seruca R. The prevalence of PIK3CA mutations in gastric and colon cancer. Eur J Cancer. 2005 July; 41(11): 1649-54. doi: 10.1016/j.ejca.2005.04.022. PMID: 15994075.

In some embodiments, the dysregulation of a PIK3CA gene, a PI3Kα protein, or expression or activity or level of any of the same, includes a splice variation in a PI3Kα mRNA which results in an expressed protein that is an alternatively spliced variant of PI3Kα having at least one residue deleted (as compared to the wild type PI3Kα protein) resulting in a constitutive activity of a PI3Kα protein domain.

In some embodiments, the dysregulation of a PIK3CA gene, a PI3Kα protein, or expression or activity or level of any of the same, includes at least one point mutation in a PIK3CA gene that results in the production of a PI3Kα protein that has one or more amino acid substitutions or insertions or deletions in a PIK3CA gene that results in the production of a PI3Kα protein that has one or more amino acids inserted or removed, as compared to the wild type PI3Kα protein. In some cases, the resulting mutant PI3Kα protein has increased activity, as compared to a wild type PI3Kα protein or a PI3Kα protein not including the same mutation. In some embodiments, the compounds described herein selectively inhibit the resulting mutant PI3Kα protein relative to a wild type PI3Kα protein or a PI3Kα protein not including the same mutation.

Exemplary Sequence of Human Phosphatidylinositol
4,5-bisphosphate 3-kinase isoform
alpha (UniProtKB entry P42336)
(SEQ ID NO: 1)
MPPRPSSGEL WGIHLMPPRI LVECLLPNGM IVTLECLREA
TLITIKHELF KEARKYPLHQ LLQDESSYIF VSVTQEAERE
EFFDETRRLC DLRLFQPFLK VIEPVGNREE KILNREIGFA
IGMPVCEFDM VKDPEVQDFR RNILNVCKEA VDLRDLNSPH
SRAMYVYPPN VESSPELPKH IYNKLDKGQI IVVIWVIVSP
NNDKQKYTLK INHDCVPEQV IAEAIRKKTR SMLLSSEQLK
LCVLEYQGKY ILKVCGCDEY FLEKYPLSQY KYIRSCIMLG
RMPNLMLMAK ESLYSQLPMD CFTMPSYSRR ISTATPYMNG
ETSTKSLWVI NSALRIKILC ATYVNVNIRD IDKIYVRTGI
YHGGEPLCDN VNTQRVPCSN PRWNEWLNYD IYIPDLPRAA
RLCLSICSVK GRKGAKEEHC PLAWGNINLF DYTDTLVSGK
MALNLWPVPH GLEDLLNPIG VTGSNPNKET PCLELEFDWF
SSVVKFPDMS VIEEHANWSV SREAGFSYSH AGLSNRLARD
NELRENDKEQ LKAISTRDPL SEITEQEKDF LWSHRHYCVT
IPEILPKLLL SVKWNSRDEV AQMYCLVKDW PPIKPEQAME
LLDCNYPDPM VRGFAVRCLE KYLTDDKLSQ YLIQLVQVLK
YEQYLDNLLV RFLLKKALTN QRIGHFFFWH LKSEMHNKTV
SQRFGLLLES YCRACGMYLK HLNRQVEAME KLINLTDILK
QEKKDETQKV QMKFLVEQMR RPDFMDALQG FLSPLNPAHQ
LGNLRLEECR IMSSAKRPLW LNWENPDIMS ELLFQNNEII
FKNGDDLRQD MLTLQIIRIM ENIWQNQGLD LRMLPYGCLS
IGDCVGLIEV VRNSHTIMQI QCKGGLKGAL QFNSHTLHQW
LKDKNKGEIY DAAIDLFTRS CAGYCVATFI LGIGDRHNSN
IMVKDDGQLF HIDFGHFLDH KKKKFGYKRE RVPFVLTQDF
LIVISKGAQE CTKTREFERF QEMCYKAYLA IRQHANLFIN
LFSMMLGSGM PELQSFDDIA YIRKTLALDK TEQEALEYFM
KQMNDAHHGG WTTKMDWIFH TIKQHALN

In some embodiments, Compound 1, or a pharmaceutically acceptable thereof, is useful for treating a cancer that has been identified as having one or more PI3Kα mutations. Accordingly, provided herein are methods for treating a subject diagnosed with (or identified as having) a cancer that include administering to the subject Compound 1, or a pharmaceutically acceptable salt thereof, and one or more additional therapeutic agents. In some embodiments, Compound 1, or a pharmaceutically acceptable thereof, is capable of crossing the blood-brain barrier (BBB) and inhibiting mutant PI3Kα in the brain and/or other central nervous system (CNS) structures. In some embodiments, Compound 1, or pharmaceutically acceptable salt thereof, is capable of crossing the BBB in a therapeutically effective amount.

Also provided herein are methods for treating a subject identified or diagnosed as having a PI3Kα-associated cancer that include administering to the subject Compound 1, or a pharmaceutically acceptable salt thereof, and one or more additional therapeutic agents. In some embodiments, the subject that has been identified or diagnosed as having a PI3Kα-associated cancer through the use of a regulatory agency-approved, e.g., FDA-approved test or assay for identifying dysregulation of a PIK3CA gene, a PI3Kα protein, or expression or activity or level of any of the same, in a subject or a biopsy sample from the subject or by performing any of the non-limiting examples of assays described herein. In some embodiments, the test or assay is provided as a kit. In some embodiments, the cancer is an PI3Kα-associated cancer.

The term “regulatory agency” refers to a country's agency for the approval of the medical use of pharmaceutical agents with the country. For example, a non-limiting example of a regulatory agency is the U.S. Food and Drug Administration (FDA).

Also provided are methods for treating cancer in a subject in need thereof, the method comprising: (a) detecting a PI3Kα-associated cancer in the subject; and (b) administering to the subject Compound 1, or a pharmaceutically acceptable salt thereof, and one or more additional therapeutic agents. In some embodiments, the subject was previously treated with another anticancer treatment, e.g., at least partial resection of the tumor or radiation therapy. In some embodiments, the subject is determined to have a PI3Kα-associated cancer through the use of a regulatory agency-approved, e.g., FDA-approved test or assay for identifying dysregulation of a PIK3CA gene, a PI3Kα protein, or expression or activity or level of any of the same, in a subject or a biopsy sample from the subject or by performing any of the non-limiting examples of assays described herein. In some embodiments, the test or assay is provided as a kit. In some embodiments, the cancer is an PI3Kα-associated cancer.

Also provided are methods of treating a subject that include performing an assay on a sample obtained from the subject to determine whether the subject has a dysregulation of a PIK3CA gene, a PI3Kα protein, or expression or activity or level of any of the same, and administering (e.g., specifically or selectively administering) Compound 1, or a pharmaceutically acceptable salt thereof, and one or more additional therapeutic agents to the subject determined to have a dysregulation of a PIK3CA gene, a PI3Kα protein, or expression or activity or level of any of the same. In some embodiments of these methods, the subject was previously treated with another anticancer treatment, e.g., at least partial resection of a tumor or radiation therapy. In some embodiments, the subject is a subject suspected of having a PI3Kα-associated cancer, a subject presenting with one or more symptoms of a PI3Kα-associated cancer, or a subject having an elevated risk of developing a PI3Kα-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided is Compound 1, or a pharmaceutically acceptable salt thereof, and one or more additional therapeutic agents, for use in treating a PI3Kα-associated cancer in a subject identified or diagnosed as having a PI3Kα-associated cancer through a step of performing an assay (e.g., an in vitro assay) on a sample obtained from the subject to determine whether the subject has a dysregulation of a PIK3CA gene, a PI3Kα protein, or expression or activity or level of any of the same, where the presence of a dysregulation of a PIK3CA gene, a PI3Kα protein, or expression or activity or level of any of the same, identifies that the subject has a PI3Kα-associated cancer. Also provided is the use of Compound 1, or a pharmaceutically acceptable salt thereof, and one or more additional therapeutic agents, for the manufacture of a medicament for treating a PI3Kα-associated cancer in a subject identified or diagnosed as having a PI3Kα-associated cancer through a step of performing an assay on a sample obtained from the subject to determine whether the subject has a dysregulation of a PIK3CA gene, a PI3Kα protein, or expression or activity or level of any of the same where the presence of dysregulation of a PIK3CA gene, a PI3Kα protein, or expression or activity or level of any of the same, identifies that the subject has a PI3Kα-associated cancer. Some embodiments of any of the methods or uses described herein further include recording in the subject's clinical record (e.g., a computer readable medium) that the subject is determined to have a dysregulation of a PIK3CA gene, a PI3Kα protein, or expression or activity or level of any of the same, through the performance of the assay, should be administered Compound 1, or a pharmaceutically acceptable salt thereof, and one or more additional therapeutic agents. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is Compound 1, or a pharmaceutically acceptable salt thereof, and one or more additional therapeutic agents, for use in the treatment of a cancer in a subject in need thereof, or a subject identified or diagnosed as having a PI3Kα-associated cancer. Also provided is the use of Compound 1, or a pharmaceutically acceptable salt thereof, and one or more additional therapeutic agents, for the manufacture of a medicament for treating a cancer in a subject identified or diagnosed as having a PI3Kα-associated cancer. In some embodiments, a subject is identified or diagnosed as having a PI3Kα-associated cancer through the use of a regulatory agency-approved, e.g., FDA-approved, kit for identifying dysregulation of a PIK3CA gene, a PI3Kα protein, or expression or activity or level of any of the same, in a subject or a biopsy sample from the subject. As provided herein, a PI3Kα-associated cancer includes those described herein and known in the art.

In some embodiments, the subject has been identified or diagnosed as having a cancer with a dysregulation of a PIK3CA gene, a PI3Kα protein, or expression or activity or level of any of the same. In some embodiments, the subject has a tumor that is positive for a dysregulation of a PIK3CA gene, a PI3Kα protein, or expression or activity or level of any of the same. In some embodiments, the subject can be a subject with a tumor(s) that is positive for a dysregulation of a PIK3CA gene, a PI3Kα protein, or expression or activity or level of any of the same. In some embodiments, the subject can be a subject whose tumors have a dysregulation of a PIK3CA gene, a PI3Kα protein, or expression or activity or level of any of the same. In some embodiments, the subject is suspected of having a PI3Kα-associated cancer. In some embodiments, provided herein are methods for treating a PI3Kα-associated cancer in a subject in need of such treatment, the method comprising a) detecting a dysregulation of a PIK3CA gene, a PI3Kα protein, or the expression or activity or level of any of the same in a sample from the subject; and b) administering Compound 1, or a pharmaceutically acceptable salt thereof, and one or more additional therapeutic agents. In some embodiments, the dysregulation of a PIK3CA gene, a PI3Kα protein, or the expression or activity or level of any of the same includes one or more PI3Kα protein point mutations/insertions/deletions. Non-limiting examples of PI3Kα protein point mutations/insertions/deletions are described in Table 1 and Table 2. In some embodiments, the PI3Kα protein point mutation/insertion/deletion is H1047X, where X is any amino acid. In some embodiments, the PI3Kα protein point mutations/insertions/deletions are selected from the group consisting of E542A, E542G, E542K, E542Q, E542V, E545A, E545D, E545G, E545K, E545Q, M1043I, M1043L, M1043T, M1043V, H1047L, H1047Q, H1047R, H1047Y, and G1049R. In some embodiments, the cancer with a dysregulation of a PIK3CA gene, a PI3Kα protein, or expression or activity or level of any of the same is determined using a regulatory agency-approved, e.g., FDA-approved, assay or kit. In some embodiments, the tumor with a dysregulation of a PIK3CA gene, a PI3Kα protein, or expression or activity or level of any of the same is determined using a regulatory agency-approved, e.g., FDA-approved, assay or kit.

In some embodiments, the subject has a clinical record indicating that the subject has a tumor that has a dysregulation of a PIK3CA gene, a PI3Kα protein, or expression or activity or level of any of the same. Also provided are methods of treating a subject that include administering Compound 1, or a pharmaceutically acceptable salt thereof, and one or more additional therapeutic agents, to a subject having a clinical record that indicates that the subject has a dysregulation of a PIK3CA gene, a PI3Kα protein, or expression or activity or level of any of the same.

In some embodiments, the methods provided herein include performing an assay on a sample obtained from the subject to determine whether the subject has a dysregulation of a PIK3CA gene, a PI3Kα protein, or expression or level of any of the same. In some such embodiments, the method also includes administering to a subject determined to have a dysregulation of a PIK3CA gene, a PI3Kα protein, or expression or activity, or level of any of the same Compound 1, or a pharmaceutically acceptable salt thereof, and one or more additional therapeutic agents. In some embodiments, the method includes determining that a subject has a dysregulation of a PIK3CA gene, a PI3Kα protein, or expression or level of any of the same via an assay performed on a sample obtained from the subject. In such embodiments, the method also includes administering to a subject Compound 1, or a pharmaceutically acceptable salt thereof, and one or more additional therapeutic agents. In some embodiments, the dysregulation in a PIK3CA gene, a PI3Kα protein, or expression or activity or level of any of the same is one or more point mutation in the PIK3CA gene (e.g., any of the one or more of the PI3Kα point mutations described herein). The one or more point mutations in a PIK3CA gene can result, e.g., in the translation of a PI3Kα protein having one or more of the following amino acid substitutions, deletions, and insertions: E542A, E542G, E542K, E542Q, E542V, E545A, E545D, E545G, E545K, E545Q, M1043I, M1043L, M1043T, M1043V, H1047L, H1047Q, H1047R, H1047Y, and G1049R. The one or more mutations in a PIK3CA gene can result, e.g., in the translation of an PI3Kα protein having one or more of the following amino acids: 542, 545, 1043, and 1047 and 1049. In some embodiments, the dysregulation in a PIK3CA gene, a PI3Kα protein, or expression or activity or level of any of the same is one or more PI3Kα amino acid substitutions (e.g., any of the PI3Kα amino acid substitution described herein). Some embodiments of these methods further include administering to the subject another anticancer agent (e.g., an immunotherapy).

In some embodiments, an assay used to determine whether the subject has a dysregulation of a PIK3CA gene, or a PI3Kα protein, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labeled nucleic acid probe or at least one labeled antibody or antigen-binding fragment thereof. Assays can utilize other detection methods known in the art for detecting dysregulation of a PIK3CA gene, a PI3Kα protein, or expression or activity or levels of any of the same (see, e.g., the references cited herein). In some embodiments, the sample is a biological sample or a biopsy sample (e.g., a paraffin-embedded biopsy sample) from the subject. In some embodiments, the subject is a subject suspected of having a PI3Kα-associated cancer, a subject having one or more symptoms of a PI3Kα-associated cancer, and/or a subject that has an increased risk of developing a PI3Kα-associated cancer).

In some embodiments, dysregulation of a PIK3CA gene, a PI3Kα protein, or the expression or activity or level of any of the same can be identified using a liquid biopsy (variously referred to as a fluid biopsy or fluid phase biopsy). See, e.g., Karachialiou et al., Ann. Transl. Med., 3(3):36, 2016. Liquid biopsy methods can be used to detect total tumor burden and/or the dysregulation of a PIK3CA gene, a PI3Kα protein, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a PIK3CA gene, a PI3Kα protein, or the expression or activity or level of any of the same. In some embodiments, liquid biopsies can be used to detect the presence of dysregulation of a PIK3CA gene, a PI3Kα protein, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a PIK3CA gene, a PI3Kα protein, or the expression or activity or level of any of the same.

Also provided herein is a method of inhibiting cell proliferation, comprising contacting a cell with Compound 1, and one or two independently selected additional therapeutic agents, as defined herein.

Further provided herein is a method of increasing cell death, comprising contacting a cell with Compound 1, and one or two independently selected additional therapeutic agents, as defined herein.

In some embodiments, the contacting is in vitro. In some embodiments, the contacting is in vivo. In some embodiments, the contacting is in vivo, wherein the method comprises administering Compound 1, or a pharmaceutically acceptable salt thereof, and and one or two independently selected additional therapeutic agents as defined herein, to a subject having a cell having aberrant PI3Kα activity. In some embodiments, the cell is a cancer cell. In some embodiments, the cancer cell is any cancer as described herein. In some embodiments, the cancer cell is a PI3Kα-associated cancer cell.

As used herein, the term “contacting” refers to the bringing together of indicated moieties in an in vitro system or an in vivo system. For example, “contacting” a PI3Kα protein with a compound provided herein includes the administration of a compound provided herein to an individual or subject, such as a human, having a PI3Kα protein, as well as, for example, introducing a compound provided herein into a sample containing a cellular or purified preparation containing the PI3Kα protein.

Also provided herein is a method of increasing tumor cell death in a subject, comprising administering to the subject Compound 1, or a pharmaceutically acceptable salt thereof, and one or two independently selected additional therapeutic agents, as defined herein.

### Combinations

The methods described herein relate to, inter alia, treating cancers (e.g., the cancers described herein, such as breast cancer) with Compound 1, or a pharmaceutically acceptable salt thereof, and one or more additional therapeutic agents.

Such additional therapeutic agents include additional therapeutic molecules (e.g., small molecules or antibodies), as well as radiation therapy (or radiotherapy) and surgery, such as open surgery or minimally invasive surgery. Compounds of Formula (I), or pharmaceutically acceptable salts thereof, such as Compound 1, therefore may also be useful as adjuvants to cancer treatment, that is, they can be used in combination with one or more additional therapies or therapeutic agents, for example, a chemotherapeutic agent that works by the same or by a different mechanism of action.

In some embodiments, a compound of Formula (I), or a pharmaceutically acceptable salt thereof, (e.g., Compound 1, or a pharmaceutically acceptable salt thereof) can be used prior to administration of one or more independently selected additional therapeutic agents or additional therapy. For example, a subject in need thereof can be administered one or more doses of a compound of Formula (I), or a pharmaceutically acceptable salt thereof, (e.g., Compound 1, or a pharmaceutically acceptable salt thereof) for a period of time and then undergo at least partial resection of the tumor. In some embodiments, the treatment with one or more doses of a compound of Formula (I), or a pharmaceutically acceptable salt thereof, (e.g., Compound 1, or a pharmaceutically acceptable salt thereof) reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof, (e.g., Compound 1, or a pharmaceutically acceptable salt thereof) for a period of time and under one or more rounds of radiation therapy. In some embodiments, the treatment with one or more doses of a compound of Formula (I), or a pharmaceutically acceptable salt thereof, (e.g., Compound 1, or a pharmaceutically acceptable salt thereof) reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a multi-kinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a multi-kinase inhibitor, immunotherapy, or radiation (e.g., radioactive iodine)). In some embodiments, a subject has a cancer (e.g., a locally advanced or metastatic tumor) that has no standard therapy. In some embodiments, a subject is PI3Kα inhibitor naïve. For example, the subject is naïve to treatment with a selective PI3Kα inhibitor. In some embodiments, a subject is not PI3Kα inhibitor naïve. In some embodiments, a subject is kinase inhibitor naïve. In some embodiments, a subject is not kinase inhibitor naïve. In some embodiments, a subject has undergone prior therapy. For example, treatment with a multi-kinase inhibitor (MKI) or another PI3K inhibitor, such as buparlisib (BKM120), alpelisib (BYL719), WX-037, idelalisib, duvelisib, copanlisib, umbralisib, (ALIQOPA™, BAY80-6946), dactolisib (NVP-BEZ235, BEZ-235), taselisib (GDC-0032, RG7604), sonolisib (PX-866), fimepinostat (CUDC-907), bimiralisib (PQR309), ZSTK474, SF1126, AZD8835, inavolisib (GDC-0077), ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, paxalisib (GDC-0084, RG7666), VS-5584 (SB2343), PKI-402, wortmannin, LY294002, PI-103, rigosertib (ON-01910 sodium salt), voxtalisib (XL-765), LY2023414, SAR260301, KIN-193 (AZD-6428), acalisib (GS-9820), AMG319, or GSK2636771.

In some embodiments of any the methods described herein, the compound of Formula (I) (or a pharmaceutically acceptable salt thereof) is administered in combination with a therapeutically effective amount of at least one additional therapeutic agent selected from one or more additional therapies or therapeutic (e.g., chemotherapeutic) agents.

Non-limiting examples of additional therapeutic agents include: other PI3Kα-targeted therapeutic agents (i.e., other PI3Kα inhibitors), EGFR inhibitors, VEGFR inhibitors/VEGF inhibitors, HER2 inhibitors, MEK pathway targeted therapeutic agents (including RAS pathway targeted therapeutic agents, which includes mTOR modulators/inhibitors, as described herein), SHP2 inhibitors, ULK inhibitors, CDK4/6 inhibitors, NTRK/ROS inhibitors, ALK inhibitors, RET inhibitors, MET inhibitors, PARP inhibitors, PIM (e.g., PIM1 and PIM3) inhibitors, other kinase inhibitors (e.g., Trk inhibitors or multi-kinase inhibitors), KAT6A inhibitors, farnesyl transferase inhibitors, aromatase inhibitors, selective estrogen receptor modulators or degraders (SERMs/SERDs, including ERα inhibitors or ERα degraders), vinca alkaloids, anti-metabolites, anti-androgens (e.g., androgen receptor (AR) antagonists, AR degraders, AR modulators), alkylating agents, checkpoint inhibitors, modulators of the apoptosis pathway, cytotoxic chemotherapeutics (also called antineoplastic chemotherapeutics), angiogenesis-targeted therapies (e.g., angiogenesis inhibitors), immune-targeted agents (including immunotherapy), radiotherapy, glucocorticoids, antidiarrheal agents (such as loperamide and diphenoxylate-atropine), antihistamines, and retinoic acid.

As used herein, a PIM inhibitor is any inhibitor of a Provirus Integration site for Moloney leukemia virus kinase (also sometimes referred to as Proviral Insertion site in Murine leukemia virus protein kinase), e.g., PIM1, PIM2, and PIM3, and any isoforms thereof (e.g., PIM-1L (molecular mass of 44 kDa) and PIM-1S (molecular mass of 33 kDa)). PIM kinases regulate cell proliferation, survival, metabolism, cellular trafficking and signaling and are overexpressed in a number of human cancers. Non-limiting examples of PIM1 inhibitors include A47, Abemaciclib (Verzenio; NCT03905889), AZD1208 (NCT01588548), AZD1897, ETH-155008, ETP-390101, ETP-45299, ETP-47551, INCB053914 (Uzansertib), JP11646, K00135, K00486, LGB321, LGH447 (PIM447), PIM447, SEL24/MEN1703 (SEL24-B489), SGI-1776, and TP-3654 (See, Belon and Nicot (2023) Mol. Cancer 22(1): 18; Mahata S., et al. (2022) Med. Oncol. 39(5): 74; Asasti V., et al. (2019) Eur J Med Chem. 172:95-108; Le X., et al. (2016) Cancer Discov. 6(10): 1134-47; Keeton E K, et al. (2014) Blood 123: 905-13; Garcia P., et al. (2013) ASH 122:1666; Grundler R, et al. (2009) J Exp Med. 206(9):1957-70; Pogacic V., et aL, (2007) Cancer Res. 67(14):6916-6924).

Exemplary SHP2 inhibitors include JAB-3312, SHP099, SHP099 hydrochloride, SHP504, RMC-3943, AS1949490, SHP394, SHP389, and RMC-4630.

In some embodiments, the SHP2 inhibitor is RMC-4630.

Exemplary ULK inhibitors include ULK-101, SBP-7455, SBI-0206965, ULK1-IN-2, MRT68921, MRT68921 dihydrochloride, MRT67307, MRT67307 hydrochloride, XST-14, and GW406108X (CW108X).

Exemplary NTRK/ROS inhibitors include entrectinib (NMS-E628, RXDX-101, ROZLYTREK®), taletrectinib (DS-6051b, AB-106), or repotrectinib (TPX-0005),

Exemplary ALK inhibitors include crizotinib (XALKORI®, PF-02341066), ceritinib (ZYKADIA®, LDK-378), alectinib (ALECENSA®, CH5424802, RO5424802, AF802), brigatinib (ALUNBRIG®, AP-26113), lorlatinib (LORBRENA®, PF-06463922), entrectinib (NMS-E628, RXDX-101, ROZLYTREK®), ASP3026, TSR-011, PF-06463922, ensartinib (X-396). or CEP-37440.

Exemplary RET inhibitors include selpercatinib (RETEVMO®, LOXO-292), zeteletinib (BOS-172738, DS-5010), GSK3179106, amuvatinib hydrochloride (MP470 hydrochloride, HPK 56 hydrochloride), TPX-0046, or pralsetinib (GAVRETO®, BLU-667).

Exemplary MET inhibitors include capmatinib (TABRECTA®, INC280; INCB28060), tepotinib (TEPMETKO®), tivantinib (ARQ197), savolitinib (ORPATHYS®, Volitinib, HMPL-504, AZD-6094), foretinib (XL880, GSK1363089, GSK089, EXEL-2880), pamufetinib (TAS-115), c-Met-IN-2, PHA-665752, SU11274, SYN1143, or amuvatinib hydrochloride (MP470 hydrochloride, HPK 56 hydrochloride).

Exemplary TRK or multi-targeted kinase inhibitors include altiratinib (DCC-2701), CH7057288, larotrectinib (VITRAKVI®), entrectinib, ANA-12, repotrectinib (TPX-0005), sitravatinib (MGCD516, MG-516), lestaurtinib (CEP-701, KT-5555), tyrphostin AG 879 (AG 879), and selitrectinib (LOXO-195).

Exemplary tyrosine kinase inhibitors include axitinib (INLYTA®), dasatinib (SPRYCEL®), erlotinib (TARCEVA®), imatinib (GLIVEC), nilotinib (TASIGNA®), pazopanib (VOTRIENT®), sunitinib (SUTENT®), and vemurafenib

Exemplary vinca alkaloids include vinorelbine, vinblastine, vincristine, vindesine, and vinflunine.

Exemplary antimetabolites include methotrexate, 5-fluorouracil (5-FU), 6-mercaptopurine (6-MP), capecitabine (XELODA®), floxuridine, cytarabine (ARA-C®), fludarabine, gemcitabine (GEMZAR®), hydroxycarbamide, pemetrexed (ALIMTA®), phototrexate, decitabine, vidaza, DFP-11207, and RX-3117, TAS-114

Exemplary alkylating agents include cyclophosphamide, lomustine, carmustine, streptozocin, bendamustine, chlorambucil, cyclophosphamide, ifosfamide, mechlorethamine, melphalan, chlorambucil, melphalan, busulfan, dacarbazine, temozolomide, altretamine, thiotepa, carboplatin, cisplatin, lurbinectedin, trabectedin, carmustine, oxaliplatin, and oxaliplatin.

Exemplary checkpoint inhibitors include nivolumab, pembrolizumab, cemiplimab, atezolizumab, durvalumab, avelumab, ipilimumab, LAG525 (IMP701), REGN3767 (R3767), BI 754,091, tebotelimab (MGD013), eftilagimod alpha (IMP321), FS118, MBG453, Sym023, TSR-022, MGC018, FPA150, EOS100850, AB928, CPI-006, monalizumab, COM701, CM24, NEO-201, defactinib, PF-04136309, MSC-1, Hu5F9-G4 (5F9), ALX148, TTI-662, RRx-001, lacnotuzumab (MCS110), LY3022855, SNDX-6352, emactuzumab (RG7155), pexidartinib (PLX3397), CAN04, canakinumab (ACZ885), BMS-986253, pepinemab (VX15/2503), trebananib, FP-1305, enapotamab vedotin (EnaV), and bavituximab.

Exemplary modulators of the apoptosis pathway include obataclax, oblimersen, ABT-737, navitoclax (ABT-263), venetoclax (ABT-199), Z-VAD-FMK, emricasan, Q-VD-Oph, Z-VAD(OH)-FMK, belnacasan (caspase-1), Z-DEVD-FMK (caspase-3), Q-VD-OphZ-IETD-FMK (caspase-8), PAC-1 (procaspase-3), nutlin-3, nutlin-3a, idasanutlin, HDM201, APR-246, CBL0137, pifithrin-α, pifithrin-μ, Z-VAD-FMK, Emricasan, Q-VD-Oph, Z-VAD(OH)-FMK, pomalidomide, lenalidomide, YM155, Venetoclax (Bel-2), S63845 (MCL-1), and A-1331852 (BCL-XL).

Exemplary cytotoxic chemotherapeutics include 5-fluorouracil, gemcitibine, methotrexate, NB1011, cyclophosphamide, dacarbazine, melphalan, tracectedin, temozolomide, doxorubicin, daunrorubincin, mitoxantrone, vinblastine, paclitaxel, docetaxel, irinotecan, etoposide, and platinum agents such as carboplatin, cisplatin, and oxaliplatin.

Exemplary topoisomerase inhibitors include etoposide, irinotecan, Camptothecin (CPT), topotecan (TPT), irinotecan, belotecan, indenoisoquinoline, phenanthridines, and indolocarbazoles. Additional examples of topoisomerase inhibitors include aminocamptothecin, CT-2106, crisnatol mesylate, DE-310, elinafide, lucanthone, MLN576, and mitindomide.

Exemplary angiogenesis-targeted therapies include bevacizumab, itraconazole, carboxyamidotriazole, TNP-470, CM101, IFN-α, IL-12, platelet factor-4, suramin, SU5416 thrombospondin, angiostatin, endostatin, 2-methoxyestradiol, tecogalan, tetrathiomolybdate, thalidomide, thrombospondin, prolactin, linomide, ramucirumab, tasquinimod, ranibizumab, sorafenib, sunitinib, pazopanib, everolimus, lenalidomide (e.g., REVLIMID®), and pomalidomide (e.g., POMALYST® or Imnovid®), marimistat, 2-methoxyestradiol (PANZEM), SU5415, SU6668, pemaxanib, sunitinib, vandetanib, vitaxin, YM598, ZD6126, and aflibercept.

In some embodiments, the angiogenesis targeted therapy is lenalidomide.

In some embodiments, the angiogenesis targeted therapy is pomalidomide.

In some embodiments, the EGFR inhibitor is osimertinib (AZD9291, merelectinib, TAGRISSO®), erlotinib (TARCEVA®), gefitinib (IRESSA®), cetuximab (ERBITUX®), necitumumab (PORTRAZZA®, IMC-11F8), neratinib (HKI-272, NERLYNX®), panitumumab (ABX-EGF, VECTIBIX®), vandetanib (CAPRELSA®), rociletinib (CO-1686), olmutinib (OLITA®, HM61713, BI-1482694), naquotinib (ASP8273), nazartinib (EGF816, NVS-816), maverlertinib (PF-06747775), icotinib (BPI-2009H), afatinib (BIBW 2992, GILOTRIF®), dacomitinib (PF-00299804, PF-804, PF-299, PF-299804), avitinib (AC00010), AC0010MA, EAI045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, β,β-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), lazertinib (YH25448), epitinib (HMPL-813), olafertinib (CK-101, RX518), MM-151, zorifertinib (AZD3759), vandetanib (ZD6474), PF-06459988, varlintinib (ASLAN001, ARRY-334543), mobocertinib (AP32788, TAK-788), pimurutamab (HLX07), befotertinib (D-0316), AEE788 (NVP-AEE788), aumolertinib (formerly almonertinib, HS-10296), avitinib, lapatinib (GW572016), pyrotinib (SHR1258), SCT200, CPGJ602, Sym004 (combination of futuximab and modotuximab), EMD 55900 (MAb-425), modotuximab (TAB-H49), futuximab (992 DS), zalutumumab, RO5083945, laprituximab emtansine (IMGN289), amivantamab (RYBREVANT™, JNJ-61186372), LY3164530, Pan-HER (Sym013), AMG 595, tuxobertinib (BDTX-189), avatinib, disruptin, CL-387785 (EKI-785, WAY-EKI 785), EGFRBi-Armed Autologous T Cells, and EGFR CAR-T Therapy. In some embodiments, the EGFR-targeted therapeutic agent is selected from gefitinib, erlotinib, afatinib, lapatinib, neratinib, osimertinib (AZD-9291, e.g., TAGRISSO®), CL-387785 (EKI-785, WAY-EKI 785), rociletinib (CO-1686), WZ4002, OMP-305B83, trastuzumab (e.g., TRAZIMERA™ HERCEPTIN®), RG-7597, and amivantanab.

In some embodiments, the EGFR inhibitor is lazertinib. In some embodiments, the EGFR inhibitor is amivantanab. In some embodiments, the EGFR inhibitor is trastuzumab.

Exemplary HER2 inhibitors include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), fam-trastuzumab deruxtecan (ENHERTU®), lapatinib, KU004, neratinib (e.g., NERLYNX®), 57 envatinib 57 govitecan-hziy (TRODELVY®) dacomitinib (e.g., VIZIMPRO®), afatinib (GILOTRIF®), tucatinib (irbinitinib, ONT-380, ARRY-380, e.g., TUKYSA™), erlotinib (e.g., TARCEVA®), pyrotinib, poziotinib, CP-724714, CUDC-101, sapitinib (AZD8931), tanespimycin (17-AAG), IPI-504, dacomitinib (PF299804, PF299), pelitinib, margetuximab, AEE-788 (NVP-AEE788), enfortumab vedotin (PADCEV®), and datopotamab deruxtecan.

In some embodiments, the HER2 inhibitor is fam-trastuzumab deruxtecan. In some embodiments, the HER2 inhibitor is 58envatinib58 govitecan-hziy. In some embodiments, the HER2 inhibitor is datopotamab deruxtecan.

Exemplary VEGFR inhibitors/VEGF inhibitors include pazopanib, sunitinib, 58 envatinib 58, cabozantinib, sorafenib, regorafenib, ponatinib, 58 envatinib axitinib, ziv-aflibercept, vandetanib, tivozanib, vatalanib, AZD-2932, aflibercept, vanucizumab, BI836880, double antiangiogenic protein (DAAP), and ramucirumab.

A “MEK pathway targeted therapeutic agent” as used herein includes any compound exhibiting inactivation activity of any protein in a MEK pathway including any protein in the RAS pathway and the RAF pathway (e.g., kinase inhibition, allosteric inhibition, inhibition of dimerization, and induction of degradation) A “RAS pathway targeted therapeutic agent” as used herein includes any compound exhibiting inactivation activity of any protein in a RAS pathway (e.g., kinase inhibition, allosteric inhibition, inhibition of dimerization, and induction of degradation). Non-limiting examples of a protein in a RAS pathway include any one of the proteins in the RAS-RAF-MAPK pathway or PI3K/AKT pathway such as RAS (e.g., KRAS, HRAS, and NRAS), RAF (ARAF, BRAF, CRAF), MEK, ERK, PI3K, AKT, and mTOR. In some embodiments, a RAS pathway modulator can be selective for a protein in a RAS pathway, e.g., the RAS pathway modulator can be selective for RAS (also referred to as a RAS modulator). In some embodiments, a RAS modulator is a covalent inhibitor. In some embodiments, a RAS pathway targeted therapeutic agent is a “KRAS pathway modulator.” A KRAS pathway modulator includes any compound exhibiting inactivation activity of any protein in a KRAS pathway (e.g., kinase inhibition, allosteric inhibition, inhibition of dimerization, and induction of degradation). Non-limiting examples of a protein in a KRAS pathway include any one of the proteins in the KRAS-RAF-MAPK pathway or PI3K/AKT pathway such as KRAS, RAF, BRAF, MEK, ERK, PI3K (i.e., other PI3K inhibitors, as described herein), AKT, and mTOR. In some embodiments, a KRAS pathway modulator can be selective for a protein in a RAS pathway, e.g., the KRAS pathway modulator can be selective for KRAS (also referred to as a KRAS modulator). In some embodiments, a KRAS modulator is a covalent inhibitor.

Non-limiting examples of RAS-targeted therapeutic agents include sotorasib (AMG 510, Lumakras®), adagrasib (MRTX849), tipifarnib (R115777, zarnestra), cysmethynil, UCM-1336, deltarasin, NHTD, RM007, RM008, gefitinib, apatinib, oncrasin-1, vismodegib (GDC-0449), N-(1-Acryloylazetidin-3-yl)-2-(5-bromo-3-(5-methoxy-1,2,3,4-tetrahydroisoquinoline-2-carbonyl)-1H-indol-1-yl) acetamide, 2-((4-((1-(2-(2,4-Dichlorophenoxy) acetyl) piperidin-4-yl) amino)-4-oxobutyl) disulfaneyl)-N,N-dimethylethan-1-aminium, ARS-1620, ARS-853, bemcentinib (BGB324), ABT-737, selumetinib (AZD6244), dactolisib (NVP-BEZ235), PPIN-1, PPIN-2, pan-RAS Inhibitor 3144 (RAS-IN-3144), deltarasin, SML-8-73-1, SML-10-70-1, 1-(2-hydroxyethyl)-4-(2-methyl-3,5-diphenylpyrazolo[1,5-a]pyrimidin-7-yl) piperazin-1-ium, (2R,4aR)-3-acryloyl-11-chloro-9-fluoro-10-(6-fluoro-2-hydroxycyclohexa-2,4-dien-1-yl)-2,6-dimethyl-2,3,4,4a-tetrahydro-1H-pyrazino [1′,2′:4,5]pyrazino[2,3-c]quinolin-5(6H)-one, NHTD, PD98059, wortmannin, talniflumate, gefitinib, CPD-0857, KY1022, KYA1797K (ab229170), 0375-0604 (DUN09716), 7773, NSC-658497, JNJ-74699157, PKF115-584 (calphostin C), Kobe0065, Kobe2602, salirasib, 3,3′-(ethylazanediyl)bis(N-phenylpropanamide), ML264, GDC-6036, LY3499446, and D-1553.

Non-limiting examples of KRAS-targeted therapeutic agents (e.g., KRAS inhibitors) include BI 1701963, sotorasib (AMG 510), ARS-3248 (JNJ-74699157), ARS1620, AZD4785 (ION651987), SML-8-73-1, SML-10-70-1, VSA9, AA12, adagrasib (MRTX-849), LY3499446, ARS853, and siG12D LODER.

Non-limiting examples of HRAS-targeted therapeutic agent (e.g., HRAS inhibitors) include tipifarnib (ZARNESTRA®). Further non-limiting examples of RAS-targeted therapeutic agents include BRAF inhibitors, MEK inhibitors, ERK inhibitors, PI3K inhibitors, AKT inhibitors, and mTOR inhibitors. In some embodiments, the

In some embodiments, the MEK inhibitor is trametinib (MEKINIST®, GSK1120212), cobimetinib (COTELLIC®), binimetinib (MEKTOVI®, MEK162), selumetinib (AZD6244), mirdametinib (PD0325901), pimasertib (MSC1936369B), SHR7390, TAK-733, RO5126766 (CH5126766), CS3006, WX-554, PD98059, CI1040 (PD184352), hypothemycin, or a combination thereof.

In some embodiments, the ERK inhibitor is amerliorex (FRI-20, ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, temuterkib (LY-3214996), rineterkib (LTT-462), KO-947, MK-8353 (SCH900353), SCH772984, ulixertinib (BVD-523), CC-90003, ravoxertinib (GDC-0994, RG-7482), ASN007, 5Z-7-oxozeaenol (FR148083, L783279, LL-Z 1640-2), 5-iodotubercidin (NSC 113939), ONC201 (TIC10), or a combination thereof.

In some embodiments, the anti-androgen is leuprolide (LUPRON®, ELIGARD®), goserelin (ZOLDEX®), triptorelin (TRELSTAR®), leuprolide mesylate (CAMCEVI®), flutamide (EULEXIN®), bicalutamide (CASODEX®), nilutamide (NILANDRON®), degarelix (FIRMAGON®), relugolix (ORGOVYX®), enzalutamide (MDV3100, XTANDI®), abiraterone (ZYTIGA®), flutamide (EULEXIN®), AR inhibitor EPI-506, apalutamide (ERLEADA®), and darolutamide (NUBEQA®).

In some embodiments, the other PI3K inhibitor is another PI3Kα inhibitor. In some embodiments, the other PI3K inhibitor is a pan-PI3K inhibitor. In some embodiments, the other PI3K inhibitor is selected from buparlisib (BKM120), alpelisib (BYL719, PIQRAY®), idelalisib, duvelisib, umbralisib, WX-037, copanlisib (ALIQOPA®, BAY80-6946), dactolisib (NVP-BEZ235, BEZ-235), taselisib (GDC-0032, RG7604), sonolisib (PX-866), fimepinostat (CUDC-907), bimiralisib (PQR309), ZSTK474, SF1126, AZD8835, inavolisib (GDC-0077), ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, paxalisib (GDC-0084, RG7666), VS-5584 (SB2343), PKI-402, wortmannin, LY294002, PI-103, rigosertib (ON-01910 sodium salt), voxtalisib (XL-765), LY2023414, SAR260301, KIN-193 (AZD-6428), acalisib (GS-9820), AMG319, GSK2636771, or a combination thereof.

In some embodiments, the AKT inhibitor is selected from miltefosine (IMPADIVO®), wortmannin, NL-71-101, H-89, GSK690693, CCT128930, capivasertib (AZD5363), ipatasertib (GDC-0068, RG7440), A-674563, A-443654, AT7867, AT13148, uprosertib (GSK2141795), afuresertib (GSK2110183), DC120, 2-[4-(2-aminoprop-2-yl)phenyl]-3-phenylquinoxaline, MK-2206, edelfosine, miltefosine, perifosine (KRX-0401), erucylphophocholine, erufosine, SR13668, OSU-A9, PH-316, PHT-427 (CS-0223), PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, miransertib (ARQ092), BAY 1125976, 3-oxo-tirucallic acid, lactoquinomycin, boc-Phe-vinyl ketone, Perifosine (D-21266), TCN, TCN-P, ONC201 (TIC10), and TAS117.

In some embodiments, the AKT inhibitor is capivasertib.

In some embodiments, the mTOR inhibitor is an analog of rapamycin. Examples of analogs of rapamycin include sapanisertib (MLN0128), vistusertib (AZD-2014), onatasertib (CC-223), CC-115, everolimus (RAD001), temsirolimus (CCI-779), ridaforolimus (AP-23573), sirolimus (rapamycin), ridaforolimus (MK-8669), everolimus (RAD001, e.g., AFINITOR® or ZORTRESS®), umirolimus, zotarolimus, and RMC-5552. In some embodiments, the mTOR inhibitor is an ATP-competitive mTOR kinase inhibitor, which compete with ATP in the catalytic site of mTOR. Examples of ATP-competitive mTOR kinase inhibitors can include torin-1, torin-2, and vistusertib. Types of ATP-competitive mTOR kinase inhibitors can include mTOR/PI3J dual inhibitors and mTORC1/mTORC2 dual inhibitors (also called TORCdIs). Examples of mTOR/PI3K dual inhibitors include dactolisib, voxtalisib, BGT226, SF1126, PKI-587, NVPBE235. Example of mTORC1/mTORC2 dual inhibitors include sapanisertib (codenamed INK128), AZD8055, and AZD2014.

Non-limiting examples of farnesyl transferase inhibitors include lonafarnib, tipifarnib, BMS-214662, L778123, L744832, and FTI-277.

In some embodiments, a chemotherapeutic agent includes an anthracycline, alkylating agents, a taxane, a platinum-based agent, mitomycin, gemcitabine, pemetrexed, eribulin (HALAVEN™), or combinations thereof.

Non-limiting examples of a taxane include paclitaxel, docetaxel, abraxane, and taxotere.

In some embodiments, the anthracycline is selected from daunorubicin, doxorubicin, epirubicin, idarubicin.

In some embodiments, the platinum-based agent is selected from carboplatin, cisplatin, oxaliplatin, nedplatin, triplatin tetranitrate, phenanthriplatin, picoplatin, satraplatin, and lobaplatin. Any of the platinum-based agents can be conjugated to a nanocarrier, such as a gold nanocluster, a gold nanoparticle, or a superparamagnetic iron oxide nanoparticles. See, for example, Zhang et al. 2022. Theranostics. 12(5):2115-2132.

Non-limiting examples of PARP inhibitors include olaparib (LYNPARZA®), talazoparib, rucaparib, niraparib, veliparib, BGB-290 (pamiparib), CEP-9883, CEP 9722, E7016 (GPI 21016), iniparib, senaparib (IMP4297), venadaparib-idience (NOV1401, IDX-1197), stenoparib (2X-121), ABT-767, atamparib (RBN-2397), talazoparib (BMN 673), olaparib (KU-0059436, AZD2281, e.g. LYNPARZA™), iniparib (BSI-201, SAR240550), rucaparib (A G-014699, PF-01367338), INO-1001, and amelparib (JPI-289).

Non-limiting examples of aromatase inhibitors include aminoglutethimide, testolactone, anastrozole, letrozole, exemestane, vorozole, formestane, and fadrozole.

Non-limiting examples of selective estrogen receptor modulators or degraders (SERMs/SERDs) include clomifene, cyclofenil, anordrin, broparoestrol, nafoxidine, ormeloxifene, raloxifene, toremifene, lasofoxifene, bazedoxifene, ospemifene, afimoxifene, enclomiphene, serophene, arzoxifene, tamoxifen, etacstil (GW-5638, DPC974), fulvestrant (FASLODEX®), brilanestrant, elacestrant (ORSERDU™), giredestrant, amcenestrant (SAR439859), camizestrant (AZD9833), rintodestrant, imlunestrant, LSZ102, LY3484356, ZN-c5, taragarestrant (D-0502), AZD9496, clotrimazole, fenticonazole, SHR9549, and palazestrant (OP-1250).

In some embodiments, the SERM/SERD is palazestrant. In some embodiments, the SERM/SERD is elacestrant. In some embodiments, the SERM/SERD is camizestrant.

Non-limiting examples of glucocorticoids include dexamethasone, beclomethasone, betamethasone, budesonide, cortisone, hydrocortisone, methylprednisolone, prednisolone, prednisone, and triamcinolone.

In some embodiments, the glucocorticoid is dexamethasone.

In some embodiments, the additional therapeutic agent is retinoic acid.

Non-limiting examples of epigenetic agents include EZH2 inhibitors (e.g., tazemetostat, such as TAZVERIK®, 3-deazaneplanocin A (DZNep or C-c3Ado), EPZ005687, Ell, GSK126, and UNC1999) and HDAC inhibitors (e.g., vorinostat (SAHA) and panobinostat (LBH589)). Non-limiting examples of HDAC inhibitors include hydroxamic acids (or hydroxamates), such as trichostatin A, vorinostat (SAHA), belinostat (PXD101), LAQ824, and panobinostat (LBH589); cyclic tetrapeptides, such as trapoxin B, and depsipeptides; benzamides, such as entinostat (MS-275), tacedinaline (CI994), and mocetinostat (MGCD0103); electrophilic ketones; and aliphatic acid compounds such as phenylbutyrate and valproic acid.

In some embodiments, the epigenetic agent is an EZH2 inhibitor. In some embodiments, the epigenetic agent is tazemetostat. In some embodiments, the EZH2 inhibitor is tazemetostat.

In some embodiments, the epigenetic agent is a HDAC inhibitor. In some embodiments, the epigenetic agent is vorinostat. In some embodiments, the epigenetic agent is panobinostat. In some embodiments, the HDAC inhibitor is vorinostat. In some embodiments, the HDAC inhibitor is panobinostat.

Non-limiting examples of KAT6A inhibitors include WM-8014, PF-07248144, CTx-648 (PF-9363), and CTX-0124143. In some embodiments, the KAT6A inhibitor is WM-8014. In some embodiments, the KAT6A inhibitor is PF-07248144. In some embodiments, the KAT6A inhibitor is CTx-648. In some embodiments, the KAT6A inhibitor is CTX-0124143.

Non-limiting examples of immunotherapy include immune checkpoint therapies, atezolizumab (TECENTRIQ®), albumin-bound paclitaxel. Non-limiting examples of immune checkpoint therapies include inhibitors that target CTLA-4, PD-1, PD-L1, BTLA, LAG-3, A2AR, TIM-3, B7-H3, VISTA, IDO, and combinations thereof. In some embodiments the CTLA-4 inhibitor is ipilimumab (YERVOY®). In some embodiments, the PD-1 inhibitor is selected from nivolumab (OPDIVO®), pembrolizumab (KEYTRUDA®), cemiplimab (LIBTAYO®), atezolizumab (TECENTRIQ®), durvalumab (IMFINZI®), avelumab (BAVENCIO®), dostarlimab (JEMPERLI®), retifanlimab (ZYNYZ®), vopratelimab (JTX-4014), spartalizumab (PDR001), camrelizumab (SHR1210), sintilimab (IBI308), tislelizumab (BGB-A317), toripalimab (JS 001), INCMGA00012 (MGA012), AMP-224, AMP-514 (MEDI0680), or Acrixolimab (YBL-006). In some embodiments, the PD-1 inhibitor is selected from pembrolizumab (KEYTRUDA®), nivolumab (OPDIVO®), cemiplimab (LIBTAYO®), or combinations thereof. In some embodiments, the PD-L1 inhibitor is selected from atezolizumab (TECENTRIQ®), avelumab (BAVENCIO®), durvalumab (IMFINZI®), or combinations thereof. In some embodiments, the LAG-3 inhibitor is leramilimab (IMP701, LAG525). In some embodiments, the A2AR inhibitor is ciforadenant (CPI-444). In some embodiments, the TIM-3 inhibitor is sabatolimab (MBG453). In some embodiments, the B7-H3 inhibitor is enoblituzumab. In some embodiments, the VISTA inhibitor is onvatilimab (JNJ-61610588). In some embodiments, the IDO inhibitor is indoximod. See, for example, Table 1 of Marin-Acevedo, et al., J Hematol Oncol. 11: 39 (2018), which is incorporated in its entirety herein.

In some embodiments, the CDK4/6 inhibitor is selected from palbociclib (IBRANCE®, TQB3616, PD-0332991), ribociclib (KISQALI®), abemaciclib (VERZENIO®), voruciclib (P1446A-05), trilaciclib, dalpiciclib (SHR6390), roniciclib (BAY1000394), dinaciclib, flavopiridol (alvociib, L868275, HMR-1275), roscovitine (R-roscovitine, CYC202, seliciclib), riviciclib (P276-00, P276), AT7519, TG02 (SB1317), RGB-286638, dinaciclib (SCH 727965), PHA-793887, ZK-304709, xytocydine, SNSO32 (BMS-387032), R547 (R₀ 4584820), RGB286147, prvalanol A (NG60), meriolin 3, JNJ7706621, indirubin, AZD-5438, 10Z-hymenialdisine, AGO24322, PF-06873600, and KIN-8741.

In some embodiments, the CDK4/6 inhibitor is KIN-8741. In some embodiments, the CDK4/6 inhibitor is palbociclib. In some embodiments, the CDK4/6 inhibitor is ribociclib. In some embodiments, the CDK4/6 inhibitor is trilaciclib. In some embodiments, the CDK4/6 inhibitor is dalpiciclib. In some embodiments, the CDK4/6 inhibitor is voruciclib. In some embodiments, the CDK4/6 inhibitor is roniciclib. In some embodiments, the CDK4/6 inhibitor is dinaciclib.

In some embodiments, the additional therapy or therapeutic agent is selected from fulvestrant, capecitabine, trastuzumab, ado-trastuzumab emtansine, pertuzumab, paclitaxel, nab-paclitaxel, enzalutamide, olaparib, pegylated liposomal doxorubicin (PLD), trametinib, palbociclib (IBRANCE®), buparlisib, sotrastaurin (AEB071), everolimus, exemestane, cisplatin, letrozole, ganitumab (AMG 479), LSZ102, ribociclib (LEE011), cetuximab, luminespib (NVP-AUY922, AUY922), infigratinib (BGJ398), binimetinib (MEK162, ARRY-162, ARRY-438162), LJM716, PIM447 (LGH447, LGB321), imatinib, gemcitabine, encorafenib (LGX818), and amcenestrant.

In some embodiments, the additional therapeutic agent is everolimus. In some embodiments, additional therapeutic agents may also be administered to treat potential side-effects for particular anticancer therapies and/or as palliative therapy, for example, opioids and corticosteroids. In some embodiments, the additional therapy or therapeutic agent described herein is selected from the group consisting of a glucagon-like peptide-1 (GLP-1) receptor agonist, a sodium-glucose transport protein 2 (SGLT-2) inhibitor, a dipeptidyl peptidase 4 (DPP-4) inhibitor, metformin, and combinations thereof.

Non-limiting examples of GLP-1 receptor agonists include liraglutide (NN2211, e.g., VICTOZA®,), dulaglutide (LY2189265, e.g., TRULICITY®), exenatide (e.g., BYETTA®, BYDUREON®, Exendin-4), taspoglutide, lixisenatide (e.g., LYXUMIA®), albiglutide (e.g., TANZEUM®), semaglutide (e.g., OZEMPIC®, RYBELSUS®), ZP2929, NNC0113-0987 (QBR110395), BPI-3016, and TT401.

Non-limiting examples of SGLT-2 inhibitors include bexagliflozin, canagliflozin (e.g., INVOKANA®), dapagliflozin (e.g., FARXIGA®), empagliflozin (e.g., JARDIANCE®), ertugliflozin (e.g., STEGLATRO™), ipragliflozin (e.g., SUGLAT®), luseogliflozin (e.g., LUSEFI®), remogliflozin, serfliflozin, licofliglozin, sotagliflozin (e.g., ZYNQUISTA™), and tofogliflozin.

Non-limiting examples of DPP-4 inhibitors include, sitagliptin (e.g., JANUVIA®), vildagliptin, saxagliptin (e.g., ONGLYZA®), linagliptin (e.g., TRADJENDA®), gemigliptin, anagliptin, teneligliptin, alogliptin, trelagliptin (e.g., NESINA®), omarigliptin, evogliptin, and dutogliptin.

In some embodiments, the additional therapeutic agent is metformin. In some embodiments, the methods described herein further comprise administering a therapeutically effective amount of metformin to the subject.

In some embodiments, the method comprises administering Compound 1, or a pharmaceutically acceptable salt thereof, and one additional therapeutic agent, for example, an aromatase inhibitor, a CDK4/6 inhibitor, a SERM/SERD, radiation therapy, an anti-HER2 antibody or antibody-drug conjugate (ADC) thereof, an immunotherapy, a checkpoint inhibitor (e.g., an anti-PD-1 or PD-L1 antibody, an anti-CLTA4 antibody), VEGFR inhibitors/VEGF inhibitors, KAT6A inhibitors (also called MOZ inhibitors or MYST3 inhibitors), PI3Kα inhibitors, MEK pathway targeted therapeutic agents (including RAS pathway targeted therapeutic agents, which includes mTOR inhibitors, as described herein), SHP2 inhibitors, ULK inhibitors, NTRK/ROS inhibitors, ALK inhibitors, RET inhibitors, MET inhibitors, PARP inhibitors, PIM (e.g., PIM1 and PIM3) inhibitors, other kinase inhibitors (e.g., Trk inhibitors or multi-kinase inhibitors), farnesyl transferase inhibitors, vinca alkaloids, anti-metabolites, anti-androgens, alkylating agents, checkpoint inhibitors, modulators of the apoptosis pathway; cytotoxic chemotherapeutics, angiogenesis-targeted therapies, immune-targeted agents including immunotherapy, or an anti-EGFR antibody.

In some embodiments, the additional therapeutic agent is an antibody or ADC as described herein. In some embodiments, the antibody is daratumumab (e.g., DARZALEX®).

In some embodiments, the method comprises administering Compound 1, or a pharmaceutically acceptable salt thereof, and one additional therapeutic agent, for example, a HER2 inhibitor, a SERM/SERD, a CDK4/6 inhibitor, a MEK inhibitor, a checkpoint inhibitor (e.g., an anti-PD-1 or PD-L1 antibody, an anti-CLTA4 antibody), a multi-kinase inhibitor, and a PI3K inhibitor.

In some embodiments, the method comprises administering Compound 1, or a pharmaceutically acceptable salt thereof, and one additional therapeutic agent, for example, trastuzumab, pertuzumab, trastuzumab emtansine, fam-trastuzumab deruxtecan, lapatinib, neratinib, dacomitinib, afatinib, tucatinib, erlotinib, pyrotinib, tanespimycin, dacomitinib, pelitinib, margetuximab, clomifene, cyclofenil, broparoestrol, ormeloxifene, raloxifene, toremifene, lasofoxifene, bazedoxifene, ospemifene, enclomiphene, serophene, tamoxifen, fulvestrant, elacestrant, camizestrant, rintodestrant, clotrimazole, fenticonazole, nivolumab, pembrolizumab, cemiplimab, atezolizumab, durvalumab, avelumab, ipilimumab, palbociclib, ribociclib, abemaciclib, trilaciclib, dalpiciclib, trametinib, cobimetinib, binimetinib, selumetinib, mirdametinib, pimasertib, alpelisib, idelalisib, duvelisib, copanlisib, umbralisib, ribociclib, triaciclib, dalpiciclib, voruciclib, and roniciclib.

In some embodiments, the additional therapeutic agent is fulvestrant.

In some embodiments, the additional therapeutic agent is lapatinib.

In some embodiments, the additional therapeutic agent is abemaciclib.

In some embodiments, the additional therapeutic agent is trametinib.

In some embodiments, the additional therapeutic agent is binimetinib.

In some embodiments, the additional therapeutic agent is alpelisib.

In some embodiments, the additional therapeutic agent is palbociclib.

In some embodiments, the additional therapeutic agent is ribociclib.

In some embodiments, the additional therapeutic agent is trilaciclib.

In some embodiments, the additional therapeutic agent is dalpiciclib.

In some embodiments, the additional therapeutic agent is voruciclib.

In some embodiments, the additional therapeutic agent is roniciclib.

In some embodiments, the additional therapeutic agent is dinaciclib.

In some embodiments, the additional therapeutic agents are palbociclib and clomifene.

In some embodiments, the additional therapeutic agents are palbociclib and cyclofenil.

In some embodiments, the additional therapeutic agents are palbociclib and anordrin.

In some embodiments, the additional therapeutic agents are palbociclib and broparoestrol.

In some embodiments, the additional therapeutic agents are palbociclib and nafoxidine.

In some embodiments, the additional therapeutic agents are palbociclib and ormeloxifene.

In some embodiments, the additional therapeutic agents are palbociclib and raloxifene.

In some embodiments, the additional therapeutic agents are palbociclib and toremifene.

In some embodiments, the additional therapeutic agents are palbociclib and lasofoxifene.

In some embodiments, the additional therapeutic agents are palbociclib and bazedoxifene.

In some embodiments, the additional therapeutic agents are palbociclib and ospemifene.

In some embodiments, the additional therapeutic agents are palbociclib and afimoxifene.

In some embodiments, the additional therapeutic agents are palbociclib and enclomiphene.

In some embodiments, the additional therapeutic agents are palbociclib and serophene.

In some embodiments, the additional therapeutic agents are palbociclib and arzoxifene.

In some embodiments, the additional therapeutic agents are palbociclib and tamoxifen.

In some embodiments, the additional therapeutic agents are palbociclib and etacstil.

In some embodiments, the additional therapeutic agents are palbociclib and fulvestrant.

In some embodiments, the additional therapeutic agents are palbociclib and brilanestrant.

In some embodiments, the additional therapeutic agents are palbociclib and elacestrant.

In some embodiments, the additional therapeutic agents are palbociclib and giredestrant.

In some embodiments, the additional therapeutic agents are palbociclib and amcenestrant.

In some embodiments, the additional therapeutic agents are palbociclib and camizestrant.

In some embodiments, the additional therapeutic agents are palbociclib and rintodestrant.

In some embodiments, the additional therapeutic agents are palbociclib and imlunestrant.

In some embodiments, the additional therapeutic agents are palbociclib and LSZ102.

In some embodiments, the additional therapeutic agents are palbociclib and LY3484356.

In some embodiments, the additional therapeutic agents are palbociclib and ZN-c5.

In some embodiments, the additional therapeutic agents are palbociclib and taragarestrant.

In some embodiments, the additional therapeutic agents are palbociclib and AZD9496.

In some embodiments, the additional therapeutic agents are palbociclib and clotrimazole.

In some embodiments, the additional therapeutic agents are palbociclib and fenticonazole.

In some embodiments, the additional therapeutic agents are palbociclib and SHR9549.

In some embodiments, the additional therapeutic agents are palbociclib and palazestrant.

In some embodiments, the additional therapeutic agents are ribociclib and clomifene.

In some embodiments, the additional therapeutic agents are ribociclib and cyclofenil.

In some embodiments, the additional therapeutic agents are ribociclib and anordrin.

In some embodiments, the additional therapeutic agents are ribociclib and broparoestrol.

In some embodiments, the additional therapeutic agents are ribociclib and nafoxidine.

In some embodiments, the additional therapeutic agents are ribociclib and ormeloxifene.

In some embodiments, the additional therapeutic agents are ribociclib and raloxifene.

In some embodiments, the additional therapeutic agents are ribociclib and toremifene.

In some embodiments, the additional therapeutic agents are ribociclib and lasofoxifene.

In some embodiments, the additional therapeutic agents are ribociclib and bazedoxifene.

In some embodiments, the additional therapeutic agents are ribociclib and ospemifene.

In some embodiments, the additional therapeutic agents are ribociclib and afimoxifene.

In some embodiments, the additional therapeutic agents are ribociclib and enclomiphene.

In some embodiments, the additional therapeutic agents are ribociclib and serophene.

In some embodiments, the additional therapeutic agents are ribociclib and arzoxifene.

In some embodiments, the additional therapeutic agents are ribociclib and tamoxifen.

In some embodiments, the additional therapeutic agents are ribociclib and etacstil.

In some embodiments, the additional therapeutic agents are ribociclib and fulvestrant.

In some embodiments, the additional therapeutic agents are ribociclib and brilanestrant.

In some embodiments, the additional therapeutic agents are ribociclib and elacestrant.

In some embodiments, the additional therapeutic agents are ribociclib and giredestrant.

In some embodiments, the additional therapeutic agents are ribociclib and amcenestrant.

In some embodiments, the additional therapeutic agents are ribociclib and camizestrant.

In some embodiments, the additional therapeutic agents are ribociclib and rintodestrant.

In some embodiments, the additional therapeutic agents are ribociclib and imlunestrant.

In some embodiments, the additional therapeutic agents are ribociclib and LSZ102.

In some embodiments, the additional therapeutic agents are ribociclib and LY3484356.

In some embodiments, the additional therapeutic agents are ribociclib and ZN-c5.

In some embodiments, the additional therapeutic agents are ribociclib and taragarestrant.

In some embodiments, the additional therapeutic agents are ribociclib and AZD9496.

In some embodiments, the additional therapeutic agents are ribociclib and clotrimazole.

In some embodiments, the additional therapeutic agents are ribociclib and fenticonazole.

In some embodiments, the additional therapeutic agents are ribociclib and SHR9549.

In some embodiments, the additional therapeutic agents are ribociclib and palazestrant.

In some embodiments, the additional therapeutic agents are abemaciclib and clomifene.

In some embodiments, the additional therapeutic agents are abemaciclib and cyclofenil.

In some embodiments, the additional therapeutic agents are abemaciclib and anordrin.

In some embodiments, the additional therapeutic agents are abemaciclib and broparoestrol.

In some embodiments, the additional therapeutic agents are abemaciclib and nafoxidine.

In some embodiments, the additional therapeutic agents are abemaciclib and ormeloxifene.

In some embodiments, the additional therapeutic agents are abemaciclib and raloxifene.

In some embodiments, the additional therapeutic agents are abemaciclib and toremifene.

In some embodiments, the additional therapeutic agents are abemaciclib and lasofoxifene.

In some embodiments, the additional therapeutic agents are abemaciclib and bazedoxifene.

In some embodiments, the additional therapeutic agents are abemaciclib and ospemifene.

In some embodiments, the additional therapeutic agents are abemaciclib and afimoxifene.

In some embodiments, the additional therapeutic agents are abemaciclib and enclomiphene.

In some embodiments, the additional therapeutic agents are abemaciclib and serophene.

In some embodiments, the additional therapeutic agents are abemaciclib and arzoxifene.

In some embodiments, the additional therapeutic agents are abemaciclib and tamoxifen.

In some embodiments, the additional therapeutic agents are abemaciclib and etacstil.

In some embodiments, the additional therapeutic agents are abemaciclib and fulvestrant.

In some embodiments, the additional therapeutic agents are abemaciclib and brilanestrant.

In some embodiments, the additional therapeutic agents are abemaciclib and elacestrant.

In some embodiments, the additional therapeutic agents are abemaciclib and giredestrant.

In some embodiments, the additional therapeutic agents are abemaciclib and amcenestrant.

In some embodiments, the additional therapeutic agents are abemaciclib and camizestrant.

In some embodiments, the additional therapeutic agents are abemaciclib and rintodestrant.

In some embodiments, the additional therapeutic agents are abemaciclib and imlunestrant.

In some embodiments, the additional therapeutic agents are abemaciclib and LSZ102.

In some embodiments, the additional therapeutic agents are abemaciclib and LY3484356.

In some embodiments, the additional therapeutic agents are abemaciclib and ZN-c5.

In some embodiments, the additional therapeutic agents are abemaciclib and taragarestrant.

In some embodiments, the additional therapeutic agents are abemaciclib and AZD9496.

In some embodiments, the additional therapeutic agents are abemaciclib and clotrimazole.

In some embodiments, the additional therapeutic agents are abemaciclib and fenticonazole.

In some embodiments, the additional therapeutic agents are abemaciclib and SHR9549.

In some embodiments, the additional therapeutic agents are abemaciclib and palazestrant.

the method comprises administering Compound 1, or a pharmaceutically acceptable salt thereof, and two additional independently selected therapeutic agents, for example, a HER2 inhibitor, a SERM/SERD, a CDK4/6 inhibitor, a MEK inhibitor, a checkpoint inhibitor (e.g., an anti-PD-1 or PD-L1 antibody, an anti-CLTA4 antibody), a multi-kinase inhibitor, and a PI3K inhibitor.

In some embodiments, the method comprises administering Compound 1, or a pharmaceutically acceptable salt thereof, and two additional independently selected therapeutic agents, for example, trastuzumab, pertuzumab, trastuzumab emtansine, fam-trastuzumab deruxtecan, lapatinib, neratinib, dacomitinib, afatinib, tucatinib, erlotinib, pyrotinib, tanespimycin, dacomitinib, pelitinib, margetuximab, clomifene, cyclofenil, broparoestrol, ormeloxifene, raloxifene, toremifene, lasofoxifene, bazedoxifene, ospemifene, enclomiphene, serophene, tamoxifen, fulvestrant, elacestrant, camizestrant, rintodestrant, clotrimazole, fenticonazole, nivolumab, pembrolizumab, cemiplimab, atezolizumab, durvalumab, avelumab, ipilimumab, palbociclib, ribociclib, abemaciclib, trilaciclib, dalpiciclib, trametinib, cobimetinib, binimetinib, selumetinib, mirdametinib, pimasertib, alpelisib, idelalisib, duvelisib, copanlisib, and umbralisib.

In some embodiments, the additional therapeutic agents are fulvestrant and lapatinib.

In some embodiments, the additional therapeutic agents are fulvestrant and trametinib.

In some embodiments, the additional therapeutic agents are fulvestrant and binimetinib.

In some embodiments, the additional therapeutic agents are fulvestrant and alpelisib.

In some embodiments, the additional therapeutic agents are fulvestrant and trilaciclib.

In some embodiments, the additional therapeutic agents are fulvestrant and dalpiciclib.

In some embodiments, the additional therapeutic agents are fulvestrant and voruciclib.

In some embodiments, the additional therapeutic agents are fulvestrant and roniciclib.

In some embodiments, the additional therapeutic agents are fulvestrant and dinaciclib.

In some embodiments, the method comprises administering Compound 1, or a pharmaceutically acceptable salt thereof, and an ERα inhibitor or degrader (e.g., a SERM or SERD).

In some embodiments, the method comprises administering Compound 1, or a pharmaceutically acceptable salt thereof, and a CDK4/6 inhibitor.

In some embodiments, the method comprises administering Compound 1, or a pharmaceutically acceptable salt thereof, and a HER2 inhibitor.

In some embodiments, the method comprises administering Compound 1, or a pharmaceutically acceptable salt thereof, and alpelisib.

In some embodiments, the method consists essentially of administering Compound 1, or a pharmaceutically acceptable salt thereof, and an ERα inhibitor or degrader.

In some embodiments, the method consists essentially of administering Compound 1, or a pharmaceutically acceptable salt thereof, and a CDK4/6 inhibitor.

In some embodiments, the method consists essentially of administering Compound 1, or a pharmaceutically acceptable salt thereof, and a HER2 inhibitor.

In some embodiments, the method consists essentially of administering Compound 1, or a pharmaceutically acceptable salt thereof, and alpelisib.

In some embodiments, the method consists essentially of administering Compound 1, or a pharmaceutically acceptable salt thereof, an ERα inhibitor or degrader, and a CDK4/6 inhibitor.

In some embodiments, the method consists essentially of administering Compound 1, or a pharmaceutically acceptable salt thereof, fulvestrant, and a CDK4/6 inhibitor.

In some embodiments, the method consists essentially of administering Compound 1, or a pharmaceutically acceptable salt thereof, fulvestrant, and palbociclib. In some embodiments, the method consists essentially of administering Compound 1, or a pharmaceutically acceptable salt thereof, fulvestrant, and ribociclib. In some embodiments, the method consists essentially of administering Compound 1, or a pharmaceutically acceptable salt thereof, fulvestrant, and abemaciclib.

In some embodiments, the method comprises administering Compound 1, or a pharmaceutically acceptable salt thereof, and fulvestrant. In some embodiments, the method comprises administering Compound 1, or a pharmaceutically acceptable salt thereof, and fulvestrant, wherein fulvestrant is administered at a dose in a range of about 250 mg to about 500 mg. In some embodiments, the method comprises administering Compound 1, or a pharmaceutically acceptable salt thereof, and fulvestrant, wherein fulvestrant is administered at a dose of about 250 mg. In some embodiments, the method comprises administering Compound 1, or a pharmaceutically acceptable salt thereof, and fulvestrant, wherein fulvestrant is administered at a dose of about 500 mg. In some embodiments, the method comprises administering Compound 1, or a pharmaceutically acceptable salt thereof, and fulvestrant, wherein fulvestrant is administered as two 5 mL injections on days 1, 15, 29 and once monthly thereafter, where each 5 mL injection includes 250 mg of fulvestrant.

In some embodiments, the method comprises administering Compound 1, or a pharmaceutically acceptable salt thereof, and lapatinib. In some embodiments, the method comprises administering Compound 1, or a pharmaceutically acceptable salt thereof, and lapatinib, wherein lapatinib is administered at a dose in a range of about 1250 mg to about 1500 mg. In some embodiments, the method comprises administering Compound 1, or a pharmaceutically acceptable salt thereof, and lapatinib, wherein lapatinib is administered at a dose of about 1250 mg. In some embodiments, the method comprises administering Compound 1, or a pharmaceutically acceptable salt thereof, and lapatinib, wherein lapatinib is administered at a dose of about 1500 mg. In some embodiments, the method comprises administering Compound 1, or a pharmaceutically acceptable salt thereof, and lapatinib, wherein lapatinib is administered as 5 tablets once daily, where each tablet includes 250 mg of lapatinib. In some embodiments, the method comprises administering Compound 1, or a pharmaceutically acceptable salt thereof, and lapatinib, wherein lapatinib is administered as 6 tablets once daily, where each tablet includes 250 mg of lapatinib.

In some embodiments, the method comprises administering Compound 1, or a pharmaceutically acceptable salt thereof, and abemaciclib. In some embodiments, the method comprises administering Compound 1, or a pharmaceutically acceptable salt thereof, and abemaciclib, wherein abemaciclib is administered at a dose in a range of about 150 mg to about 400 mg. In some embodiments, the method comprises administering Compound 1, or a pharmaceutically acceptable salt thereof, and abemaciclib, wherein abemaciclib is administered at a dose of about 150 mg. In some embodiments, the method comprises administering Compound 1, or a pharmaceutically acceptable salt thereof, and abemaciclib, wherein abemaciclib is administered at a dose of about 200 mg. In some embodiments, the method comprises administering Compound 1, or a pharmaceutically acceptable salt thereof, and abemaciclib, wherein abemaciclib is administered at a dose of about 300 mg. In some embodiments, the method comprises administering Compound 1, or a pharmaceutically acceptable salt thereof, and abemaciclib, wherein abemaciclib is administered at a dose of about 400 mg. In some embodiments, the method comprises administering Compound 1, or a pharmaceutically acceptable salt thereof, and abemaciclib, wherein abemaciclib is administered as 50 mg, 100 mg, 150 mg, or 200 mg tablets. In some embodiments, the method comprises administering Compound 1, or a pharmaceutically acceptable salt thereof, and abemaciclib, wherein 150 mg of abemaciclib is administered tablets twice daily. In some embodiments, the method comprises administering Compound 1, or a pharmaceutically acceptable salt thereof, and abemaciclib, wherein 200 mg of abemaciclib is administered tablets twice daily.

In some embodiments, the method comprises administering Compound 1, or a pharmaceutically acceptable salt thereof, and a MEK inhibitor.

In some embodiments, the method comprises administering Compound 1, or a pharmaceutically acceptable salt thereof, and trametinib. In some embodiments, the method comprises administering Compound 1, or a pharmaceutically acceptable salt thereof, and trametinib, wherein trametinib is administered at a dose in a range of about 1 mg to about 2 mg. In some embodiments, the method comprises administering Compound 1, or a pharmaceutically acceptable salt thereof, and trametinib, wherein trametinib is administered at a dose of about 1 mg, when the subject is a pediatric patient with a body weight of about 26 kg to about 37 kg. In some embodiments, the method comprises administering Compound 1, or a pharmaceutically acceptable salt thereof, and trametinib, wherein trametinib is administered at a dose of about 1.5 mg, when the subject is a pediatric patient with a body weight of about 38 kg to about 50 kg. In some embodiments, the method comprises administering Compound 1, or a pharmaceutically acceptable salt thereof, and trametinib, wherein trametinib is administered at a dose of about 2 mg, when the subject is a pediatric patient with a body weight of about 51 kg or greater. In some embodiments, the method comprises administering Compound 1, or a pharmaceutically acceptable salt thereof, and trametinib, wherein trametinib is administered at a dose of about 2 mg. In some embodiments, the method comprises administering Compound 1, or a pharmaceutically acceptable salt thereof, and trametinib, wherein trametinib is administered as 0.5 mg or 2 mg tablets. In some embodiments, the method comprises administering Compound 1, or a pharmaceutically acceptable salt thereof, and trametinib, wherein 2 mg of trametinib is administered once daily. In some embodiments, the method comprises administering Compound 1, or a pharmaceutically acceptable salt thereof, and trametinib, wherein 1.5 mg of trametinib is administered once daily. In some embodiments, the method comprises administering Compound 1, or a pharmaceutically acceptable salt thereof, and trametinib, wherein 1 mg of trametinib is administered once daily.

In some embodiments, the method comprises administering Compound 1, or a pharmaceutically acceptable salt thereof, and binimetinib. In some embodiments, the method comprises administering Compound 1, or a pharmaceutically acceptable salt thereof, and binimetinib, wherein binimetinib is administered at a dose in a range of about 30 mg to about 90 mg. In some embodiments, the method comprises administering Compound 1, or a pharmaceutically acceptable salt thereof, and binimetinib, wherein binimetinib is administered at a dose of about 30 mg. In some embodiments, the method comprises administering Compound 1, or a pharmaceutically acceptable salt thereof, and binimetinib, wherein binimetinib is administered at a dose of about 45 mg. In some embodiments, the method comprises administering Compound 1, or a pharmaceutically acceptable salt thereof, and binimetinib, wherein binimetinib is administered at a dose of about 60 mg. In some embodiments, the method comprises administering Compound 1, or a pharmaceutically acceptable salt thereof, and binimetinib, wherein binimetinib is administered at a dose of about 90 mg. In some embodiments, the method comprises administering Compound 1, or a pharmaceutically acceptable salt thereof, and binimetinib, wherein binimetinib is administered as 15 mg tablets. In some embodiments, the method comprises administering Compound 1, or a pharmaceutically acceptable salt thereof, and binimetinib, wherein 45 mg of binimetinib is administered as three 15 mg tablets twice daily. In some embodiments, the method comprises administering Compound 1, or a pharmaceutically acceptable salt thereof, and binimetinib, wherein 30 mg of binimetinib is administered as two 15 mg tablets twice daily.

In some embodiments, the method comprises administering Compound 1, or a pharmaceutically acceptable salt thereof, and palbociclib. In some embodiments, the method comprises administering Compound 1, or a pharmaceutically acceptable salt thereof, and palbociclib, wherein palbociclib is administered at a dose in a range of about 75 mg to about 125 mg. In some embodiments, the method comprises administering Compound 1, or a pharmaceutically acceptable salt thereof, and palbociclib, wherein palbociclib is administered at a dose of about 75 mg. In some embodiments, the method comprises administering Compound 1, or a pharmaceutically acceptable salt thereof, and palbociclib, wherein palbociclib is administered at a dose of about 100 mg. In some embodiments, the method comprises administering Compound 1, or a pharmaceutically acceptable salt thereof, and palbociclib, wherein palbociclib is administered at a dose of about 125 mg. In some embodiments, the method comprises administering Compound 1, or a pharmaceutically acceptable salt thereof, and palbociclib, wherein palbociclib is administered as 75 mg, 100 mg, or 125 mg tablets. In some embodiments, the method comprises administering Compound 1, or a pharmaceutically acceptable salt thereof, and palbociclib, wherein 75 mg of palbociclib is administered once daily. In some embodiments, the method comprises administering Compound 1, or a pharmaceutically acceptable salt thereof, and palbociclib, wherein 100 mg of palbociclib is administered once daily. In some embodiments, the method comprises administering Compound 1, or a pharmaceutically acceptable salt thereof, and palbociclib, wherein 125 mg of palbociclib is administered once daily. In some embodiments, the method comprises administering Compound 1, or a pharmaceutically acceptable salt thereof, and palbociclib, wherein 125 mg of palbociclib is administered once daily as a 125 mg tablet.

### Pharmaceutical Compositions

Some embodiments provide a pharmaceutical composition comprising a compound of Formula (I), or a pharmaceutically acceptable salt thereof, and one or more pharmaceutically acceptable excipients.

Some embodiments provide a pharmaceutical composition comprising Compound 1, or a pharmaceutically acceptable salt thereof, and one or more pharmaceutically acceptable excipients.

In some embodiments, the pharmaceutical composition comprises one or two additional therapeutic agents, i.e., as a fixed-dose combination. In some embodiments, the pharmaceutical composition comprises one additional therapeutic agent. In some embodiments, the pharmaceutical composition comprises two additional therapeutic agents.

### Dosing

In some embodiments, Compound 1, or a pharmaceutically acceptable salt thereof, is administered at a dose in a range of about 1 mg to about 500 mg, e.g., about 1 mg to about 450 mg, about 1 mg to about 400 mg, about 1 mg to about 350 mg, about 1 mg to about 300 mg, about 1 mg to about 250 mg, about 1 mg to about 200 mg, about 1 mg to about 150 mg, about 1 mg to about 100 mg, about 1 mg to about 50 mg, about 1 mg to about 40 mg, about 1 mg to about 30 mg, about 1 mg to about 25 mg, about 1 mg to about 20 mg, about 1 mg to about 15 mg, about 1 mg to about 10 mg, or about 1 mg to about 5 mg.

In some embodiments, Compound 1, or a pharmaceutically acceptable salt thereof, is administered at a dose in a range of about 1 mg to about 100 mg, e.g., about 1 mg to about 80 mg, about 1 mg to about 75 mg, about 1 mg to about 60 mg, about 1 mg to about 50 mg, about 1 mg to about 40 mg, about 1 mg to about 30 mg, or about 1 mg to about 20 mg.

In some embodiments, Compound 1, or a pharmaceutically acceptable salt thereof, is administered at a dose in a range of about 20 mg to about 100 mg, e.g., about 20 mg to about 80 mg, about 30 mg to about 80 mg, about 30 mg to about 70 mg, about 30 mg to about 60 mg, about 20 mg to about 50 mg, about 40 mg to about 70 mg, about 40 mg to about 60 mg, or about 45 mg to about 55 mg.

In some embodiments, Compound 1, or a pharmaceutically acceptable salt thereof, is administered at a dose in a range of about 1 mg to about 50 mg, e.g., about 1 mg to about 45 mg, about 1 mg to about 40 mg, about 1 mg to about 35 mg, about 1 mg to about 30 mg, about 1 mg to about 25 mg, about 1 mg to about 20 mg, about 1 mg to about 15 mg, about 1 mg to about 10 mg, or about 1 mg to about 5 mg.

In some embodiments, the total daily dosage of Compound 1, or a pharmaceutically acceptable salt thereof, is as described herein. In some embodiments, Compound 1, or a pharmaceutically acceptable salt thereof, is administered once per day. In some embodiments, Compound 1, or a pharmaceutically acceptable salt thereof, is administered twice per day.

In some embodiments, Compound 1, or a pharmaceutically acceptable salt thereof, is administered as a monotherapy at the dosages described herein.

In some embodiments, Compound 1, or a pharmaceutically acceptable salt thereof, is administered in combination with one or more additional therapeutic agents as described herein, at the dosages of Compound 1 described herein. In some embodiments, the dosages of the one or more additional therapeutic agents are the standard dosages of the one or more additional therapeutic agents, for example, as indicated on a regulatory agency-approved label (e.g., the USFDA or EMA).

### EXAMPLES

### Compound Preparation

### Compound 1: (R)-1-(2-aminopyrimidin-5-yl)-3-(1-(5,7-difluoro-3-methylbenzofuran-2-yl)-2,2,2-trifluoroethyl)urea

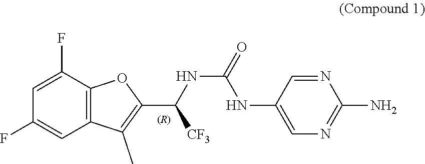

### Step 1

To a mixture of pyrimidine-2,5-diamine (3.0 g, 27.2 mmol) and NaHCO₃ (11.4 g, 135.9 mmol) in THF (300 mL) was added phenyl carbonochloridate (4.5 g, 28.5 mmol) dropwise at 0° C. The mixture was stirred at 25° C. for 4 h. After reaction, the reaction mixture was concentrated to give a residue which was purified by silica gel chromatography column (DCM/MeOH from 0-10%) to give phenyl (2-aminopyrimidin-5-yl)carbamate (2.2 g, 34%) as a brown solid. MS (ESI): mass calcd. for C₁₁H₁₀N₄O₂, 230.1, m/z found 231.1 [M+H]⁺.

### Step 2

To a solution of 1-(3,5-difluoro-2-hydroxyphenyl)ethan-1-one (20 g, 116.2 mmol) in DMF (200 mL) were added methyl 2-bromoacetate (19.4 g, 127.9 mmol) and K₂CO₃ (24.1 g, 174.4 mmol) at room temperature. The reaction mixture was stirred at room temperature for 2 h. After reaction, the insoluble material was filtered off and DBU (17.7 g, 116.2 mmol) was added into the filtrate which was stirred again at 80° C. for 2 h. After reaction, the reaction mixture was concentrated to give a residue which was purified by silica gel chromatography column (PE/DCM from 0-35%) to give methyl 5,7-difluoro-3-methylbenzofuran-2-carboxylate (8.4 g, 32%) as off-white solid. MS (ESI): mass calcd. for C₁₁H₈F₂O₃, 226.0, m/z found 227.0 [M+H]⁺.

### Step 3

To a solution of 5,7-difluoro-3-methylbenzofuran-2-carboxylate (8.1 g, 35.8 mmol) in THF (160 mL) was added LiAlH₄ (21.5 mL, 21.5 mmol, 1M in THF) at 0° C. The reaction mixture was stirred at 0° C. for 2 h. then slowly quenched with saturated aqueous potassium carbonate (150 mL) and extracted with EA (200 mL×3). The combined organic layer was washed with brine and dried with Na₂SO₄ and concentrated to give a residue which was purified by silica gel chromatography column (PE/EA from 0-30%) to give (5,7-difluoro-3-methylbenzofuran-2-yl)methanol (6.5 g, 91%) as an off-white solid. MS (ESI): mass calcd. for C₁₀H₈F₂O₂, 198.0, m/z found 181.0 [M−H₂O+H]⁺.

### Step 4

To a solution of (5,7-difluoro-3-methylbenzofuran-2-yl) methanol (6.5 g, 32.6 mmol) in ACN (65 mL) was added IBX (13.7 g, 49.0 mmol) at room temperature. The reaction mixture was stirred at 80° C. for 2 h. The insoluble material was then filtered off and the filtrate was concentrated to give a residue which was purified by silica gel chromatography column (PE/EA from 0˜15%) to give 5,7-difluoro-3-methylbenzofuran-2-carbaldehyde (6.1 g, 95%) as an off-white solid. MS (ESI): mass calcd. for C₁₀H₆F₂O₂, 196.0, m/z found 197.1 [M+H]⁺.

### Step 5

To a solution of 5,7-difluoro-3-methylbenzofuran-2-carbaldehyde (6.1 g, 31.1 mmol) in DMF (92 mL) were added trimethyl(trifluoromethyl)silane (8.89 g, 62.2 mmol) and K₂CO₃ (2.1 g, 15.5 mmol) at 0° C. The reaction mixture was stirred at room temperature for 0.5 h and then another batch of K₂CO₃ (4.3 g, 31.0 mmol) was added into the reaction mixture. The mixture was stirred at room temperature for 16 h and then H₂O (2.8 g, 115.5 mmol) was added, and the reaction mixture was further stirred at 0° C. for 1 h. The mixture was quenched with ice water and extracted with EA (200 mL×3). The combined organic layer was washed with brine and dried with Na₂SO₄ and concentrated to give a residue which was purified by silica gel chromatography column (PE/EA from 0-20%) to give 1-(5,7-difluoro-3-methylbenzofuran-2-yl)-2,2,2-trifluoroethan-1-ol (6.5 g, 78%) as light yellow oil. MS (ESI): mass calcd. for C₁₁H₇F₅O₂, 266.0, m/z found 249.1 [M−H₂O+H]⁺.

### Step 6

To a solution of 1-(5,7-difluoro-3-methylbenzofuran-2-yl)-2,2,2-trifluoroethan-1-ol (370 mg, 1.39 mmol) in ACN (10 mL) was added IBX (584 mg, 2.08 mmol). The reaction mixture was refluxed for 16 h. After reaction, the mixture was filtered and washed with EA. The filtrate was collected and concentrated to give a residue which was purified by silica gel chromatography column (PE/EA from 0-20%) to 1-(5,7-difluoro-3-methylbenzofuran-2-yl)-2,2,2-trifluoroethan-1-one (320 mg, 87%) as yellow oil. ¹H NMR (400 MHz, DMSO) δ 7.82-7.74 (m, 2H), 2.65 (s, 3H).

### Step 7

A mixture of 1-(5,7-difluoro-3-methylbenzofuran-2-yl)-2,2,2-trifluoroethan-1-one (320 mg, 1.21 mmol), hydroxylamine hydrochloride (585 mg, 8.48 mmol) and NaOAc (992 mg, 12.10 mmol) in EtOH (10 mL) was refluxed for 16 h. After reaction, the mixture was concentrated and redissolved in MeOH (10 mL) which was added Raney Ni (50 mg) and one drop of ammonia. The mixture was stirred under H₂ at room temperature for 6 h. After reaction, the mixture was filtered and the filtrate was concentrated to give a residue which was purified by silica gel chromatography column (PE/EA from 0-50%) to give 1-(5,7-difluoro-3-methylbenzofuran-2-yl)-2,2,2-trifluoroethan-1-amine (150 mg, 47%) as light yellow oil. MS (ESI): mass calcd. for C₁₁H8F₅NO, 265.0, m/z found 249.1 [M-NH₃+H]⁺.

### Step 8

To a mixture of 1-(5,7-difluoro-3-methylbenzofuran-2-yl)-2,2,2-trifluoroethan-1-amine (150 mg, 0.57 mmol), DIEA (219 mg, 1.70 mmol) in DMF (10 mL) was added phenyl (2-aminopyrimidin-5-yl)carbamate (143 mg, 0.62 mmol) at 0° C. The reaction mixture was then stirred at room temperature for 16 h then quenched with water and extracted with EA (50 mL×3). The combined organic layer was concentrated to give a residue which was purified by silica gel chromatography column (PE/EA from 0-50%) to give (rac)-1-(2-aminopyrimidin-5-yl)-3-(1-(5,7-difluoro-3-methylbenzofuran-2-yl)-2,2,2-trifluoroethyl)urea (150 mg, 66%) as light yellow oil. MS (ESI): mass calcd. for: C₁₆H₁₂F₅N₅O₂, 401.1, m/z found 402.1 [M+H]⁺.

### Step 9

(rac)1-(2-aminopyrimidin-5-yl)-3-(1-(5,7-difluoro-3-methylbenzofuran-2-yl)-2,2,2-trifluoroethyl)urea (150 mg) was separated by chiral HPLC to give the (S) enantiomer of Compound 1 (peak 1, 61 mg, 41%) and Compound 1 (peak 2.58 mg, 39%) MS (ESI): mass calcd. for C₁₆H₁₂F₅N₅O₂, 401.1, m/z found 402.1 [M+H]⁺.

### (S) Enantiomer of Compound 1 (Peak 1):

¹H NMR (400 MHz, DMSO-d6) δ 8.21 (s, 2H), 8.20 (s, 1H), 7.81 (d, J=8.0 Hz, 1H), 7.46-7.39 (m, 2H), 6.40 (s, 2H), 6.09-5.98 (m, 1H), 2.29 (s, 3H).

Compound 1 (peak 2):

¹H NMR (400 MHz, DMSO-d6) δ 8.19 (s, 2H), 8.18 (s, 1H), 7.79 (d, J=8.0 Hz, 1H), 7.46-7.34 (m, 2H), 6.37 (s, 2H), 6.03-5.97 (m, 1H), 2.27 (s, 3H).

### Assays

Homogenous Time-Resolved Fluorescence (HTRF)—pAKT-T47D

Compound 1 was assayed using homogeneous time-resolved fluorescence (HTRF).

### Materials, Reagents, and Equipment

Gibco RPMI 1640 Medium, no phenol red; Gibco RPMI 1640 Medium; Gibco Trypsin-EDTA (0.5%), no phenol red; Gibco DPBS; Trypan blue solution 0.4% (Corning); Avantor Seradigm Premium Grade Fetal Bovine Serum (FBS); Greiner 784080-384 well TC treated white plates; pAKT (Ser473) HTRF; Gibco Insulin, human recombinant, zinc solution; Gibco Recovery Cell Culture Freezing Medium; Countess II FL Automated Cell Counter (ThermoFisher); Countess II Slides (ThermoFisher); Microscope; and PHERAstar FSX Microplate Reader (BMG LABTECH, Inc.).

### Procedure

The scinamic cell line ID was T47D.1, the HTRF detection was pAKT (S473), a PI3Kα H1047R mutation was present, the seeding density was 5000, the timepoint was 1 hour, and the medium used was RPMI+10% FBS (no phenol red)+0.2 units/ml bovine insulin.

### Cell Culture Maintenance:

The cell density was not permitted to reach 100% confluence. The cells were split 1:5 when they reached ˜80% confluence.
   Cells were split twice a week (Mon and Fri).
   Cells over passage 18 were not used (˜2 months of maintenance).
   Antibiotics were not used for tissue culture maintenance or assays.

### For Freezing Cells:

1. Trypsinized cells were collected and counted. Cells were pelleted at 1000 rpm, 5 minutes and supernatant was aspirated.
2. Pelleted cells were gently resuspended at 3e6 cells/1 mL of freezing medium (Gibco Freezing Medium). For example, if there were 9e6 total cells, cell pellet was resuspended in 3 mL of freezing medium.
3. Measured aliquot of 1 mL of resuspended cells/cryovial. Cells were frozen in appropriate cell freezing container (i.e. Mr. Frosty or Corning CoolCell Freezing System) at −80° C.
4. Cells were transferred to Liquid Nitrogen Cryotank for long term storage.

### For Thawing Cells:

1. Cells were removed from liquid nitrogen tank. Cryovials were thawed in 37° C. waterbath until small “ice pellet” remained. This was then sprayed down with 70% ethanol before moving to TC/BSC hood.
2. Added 9 ml of fresh media to a 15 mL conical tube. Added 10 mL of fresh media to a T75 TC treated flask.
3. Gently transferred 1 mL of cells in freezing medium from cryovial to 15 mL conical tube containing media.
4. Centrifuged at 1000 rpm, 5 mins to pellet cells.
5. Aspirated media/freezing media.
6. Gently resuspended cell pellet in 5 mL fresh media and transferred to T75 flask with 10 mLs of fresh media. Place flasks in 37° C. incubator, 5% C02.

### Protocol

### Day 1

The procedure was as follows:
1. Prepared ARP:
   a. Stamped 12.5 nL from 10 mM source plate to destination plate using Echo. Sealed plate immediately and froze at −20° C. if it was not used on the same day.
   b. If a frozen ARP was used, the plate was thawed and spun at 1000 rpm×1 min.
2. Preparation of cells (adherent):
   a. Aspirated media from cells. Washed cells with sterile 1×PBS. Aspirated PBS and added appropriate amount of Trypsin.
   b. Once cells were fully trypsinized, added appropriate media to resuspend cells. Transferred cells to a 15 mL or 50 mL conical tube.
   c. Counted cells on the Countess II Cell Counter.
3. Plating of cells:
   a. Prepared cells at appropriate plating density. Dispensed 12 μL of diluted cells per well of a Greiner 784080-384 well TC treated white plate using a Multidrop Combi to columns 1-23. Added 12 μL of appropriate phenol free media only to column 24.
   b. Placed plates in 37° C. tissue culture incubator for appropriate treatment time (refer to “Assay” table).
4. Prepared HTRF Lysis Buffer
   a. Calculate the amount of HTRF lysis buffer master mix needed to perform the desired experiments plus any extra dead volume required for dispensing (4 μL required per well). Dilute the Blocking Reagent into 4× Lysis Buffer at a ratio of 1:25 (i.e. 0.1 mL Blocking Reagent Solution plus 2.4 mL 4× Lysis Buffer).
   b. Add 4 uL Lysis buffer master mix to all wells with sample or DMSO. Centrifuge the plates for 1 minute at 1000 rpm.
   c. Incubate at room temperature for 30 minutes.
5. Prepared HTRF Antibody
   a. Calculated the amount of HTRF antibody master mix needed to perform the desired experiments plus any extra dead volume required for dispensing (4 mL required per well). Eu Cryptate antibody and d2 antibody were added to detection buffer each at
   a ratio of 1:40 (i.e. 100 μL Eu Cryptate+100 μL d2 Cryptate+3800 μL detection buffer).
   b. 4 μL of antibody master mix was added to each well including the media only column 24.
   c. Centrifuged the plates for 1 minute at 1000 rpm. Placed lid on and created a “humidity chamber” by placing the plates into a ziplock bag with wet paper towels or something similar and incubated overnight at room temperature, keeping away from light.

### Day 2

6. Measured on the PHERAstar/Envision using the HTRF protocol. When plates were read, all wells were read.

The IC₅₀ (nM) of Compound 1 in the T47D pAKT assay was between 31 and 42 nM.

### Surface Plasmon Resonance (SPR)

PI3K binding can also be determined by SPR. SPR experiments are performed on a Biacore 8K instrument. A biotinylated recombinant PI3Kα H1047R protein containing a full-length p110-α subunit harboring the H1047R mutation with an N-terminal AviTag, complexed with a truncated p85-α subunit (amino acid residues 322-694) is used. The protein is first incubated with 1 μM wortmannin for 30 min at RT to covalently block the ATP binding site, then immobilized onto a streptavidin sensor chip by flowing the protein through the sensor chip at typically 20 g/mL concentration and 2 μL/min flow rate for 1200 seconds. Compound binding affinity is measured in the multi-cycle kinetics mode, at 90 μL/min flow rate with 90 seconds association time and 240 seconds dissociation time. The running buffer contains 50 mM Tris, pH 7.5, 150 mM NaCl, 0.01% Brij35, 1 mM DTT, 1 mM MgCl₂, 0.05% Tween-20 and 2% DMSO. Temperature is maintained at 25° C. during experiments, and data were fit into the 1:1 binding model.

### Crystallization

Crystals of the PI3Kα (H1047R)/p85α heterodimer in complex with GDC-0077 and Compound 1 were obtained at a concentration of 10 mg/ml (20 mM Tris/HCl, 150 mM NaCl, 1 mM TCEP, pH 8.0) and pre-incubated with 1.5 excess of GDC-0077 and Compound 1 (150 mM in dimethyl sulfoxide (DMSO) for 1 hour; 0.8 μl of the protein solution was then mixed with 0.8 μl of reservoir solution (0.1 M MES pH 6.8, 0.5 M NaCl, 5% (w/v) polyethylene glycol 3350) and equilibrated at 293 K over 60 μl of reservoir solution. Well-diffracting crystals were selected for data collection after 5 days. Crystals were cryoprotected in reservoir solution supplemented with 30% ethylene glycol and flash frozen in liquid nitrogen before data collection. A complete 2.9 Å data set of a PI3Kα (H1047R)/p85α/GDC-0077/Compound 1 crystal was collected at the European Synchotron Radiation Facility (ESRF, Grenoble, FR, beamline ID30a1) and the data were integrated, analyzed, and scaled by the programs XDS, Pointless, and STARANISO from within the autoPROC pipeline, respectively.

### Structure Solution and Refinement.

An isomorphous reference model of PI3Kα in complex with p85α and GDC-0077 was used as a starting model for restrained refinement of the H1047R dataset with REFMAC5. Several rounds of refinement with the programs REFMAC5 and then BUSTER resulted in the final model. Atomic displacement factors were modelled with a single isotropic B-factor per atom and a single TLS group per chain. Non-crystallographic symmetry restraints were used. The restraints for the compounds GDC-0077 and Compound 1 were generated using GRADE from Global Phasing using the big planes option. The final model consisted of two heterodimers in the asymmetric unit, both bound with Compound 1 and GDC-0077. The chain A and B heterodimer was better resolved than the other heterodimer and was therefore used in the analysis of Compound 1 binding. Statistics for the crystal structure are reported in Table 13. Images were generated using PyMOL (www.pymol.org).

TABLE 13
Compound 1 X-ray crystallography statistics

| Data collection | | |
|---|---|---|
| Resolution (Å) | 34.42-2.93 | (3.33-2.93) |
| Space group | P21 | |
| Cell dimensions | | |
| a, b, c, (Å) | 86.2, 124.5, 165.4 | |
| α, β, γ (°) | 90, 92.9, 90 | |
| No. unique reflections | 55,994 | (5600) |
| I/σ(I) | 11.5 | (1.7) |
| Completeness (%) | 94.1 | (65.3) |
| Multiplicity | 4.8 | (5.0) |
| Rₘₑₐₛ | 0.11 | (1.10) |
| Rₚᵢₘ | 0.05 | (0.49) |
| CC(1/2) | 0.998 | (0.582) |
| Resolution (Å) | 34.42-2.93 | (3.33-2.93) |
| Refinement | | |
| R_{work} | 0.210 | (0.307) |
| R_{free} | 0.248 | (0.376) |

| | |
|---|---|
| RMS deviations | |
| Bond lengths (Å) | 0.007 |
| Bond angles (°) | 0.79 |

Values in parentheses represent the highest resolution shell. RMS, root mean square.

### PI3Kα ATPase Assay

Full-length WT, M1043X, H1047X, or G1049R enzyme (1-10 nM) was incubated with vehicle or compound at room temperature for 1 hour, followed by addition of ATP (90 μM final) to initiate the enzyme reaction. Assay buffer contained 50 mM Tris, 150 mM NaCl, 0.01% Brij 35, 15 mM MgCl₂, 0.05% Tween-20, and 1 mM DTT. ADP production was measured after a 100 min incubation at room temperature, using the ADP-Glo kit (Promega #V9102).

### Kinome Selectivity Profiling

In vitro kinase profiling assay of Compound 1 was assessed across 373 kinases (KinaseProfiler™, IC₅₀Profiler™; Eurofins Cerep, Le Bois l'Évêque, France).

### Cellular Assays

For cell-line details, please see Table 14.

TABLE 14
Cell lines and plating density used for
HTRF (pAKT) and viability (CTGLO).

| | | HTRF | CTGlo |
|---|---|---|---|
| | | seeding | seeding |
| | | density | density |
| Cell Line | RRID | (384 well) | (384 well) |
| T47D | CVCL_0178 | 5000 | 1500 |
| CAL-33 | CVCL_1106 | 5000 | 1200 |
| SKBR3 | CVCL_1108 | 5000 | 1500 |
| NCI-H1048 | CVCL_1171 | 10,000 | 1500 |
| HCC1954 | CVCL_0253 | 5000 | 2000 |
| OAW42 | CVCL_2450 | 5000 | 500 |
| Detroit562 | CVCL_1259 | 5000 | 500 |
| BT-20 | CVCL_1453 | 5000 | 1000 |
| GP2d | CVCL_1615 | 5000 | 1000 |
| CAL-148 | CVCL_0553 | 5000 | 1000 |
| EFM-19 | CVCL_0033 | 5000 | 3000 |

pAKT Homogenous Time-Resolve Fluorescence (HTRF)

A 384-well phospho-AKT (S473) HTRF (PerkinElmer #64AKSPEH) assay was used for target engagement. Each well was seeded with cell numbers indicated in Supplementary Table S3 in a volume of 12.5 uL in phenol red-free media and then treated for 1 hour at 37° C., before reading on a PHERAstar plate reader.

### Cell Proliferation

Cell viability was measured using CellTiterGlo (Promega #G9243). Indicated cell lines were seeded according to Supplementary Table S3 in 50 μL of media in 384 well plates, with 72 hours of treatment at 37° C. with 5% CO₂.

Compound 1 was submitted to the Broad Institute PRISM high throughput cell viability screen (www.theprismlab.org/).

### Human Adipocyte ³H-2-Deoxyglucose Uptake

Primary human subcutaneous adipocytes were treated with test compound or vehicle for 1 hour followed by addition of 10 nM insulin and ³H-2-deoxyglucose (Zen Bio Durham, NC USA, assay #CA-25 Lot #SL0071). Cytochalasin B treatment controlled for non-specific glucose uptake. Corrected counts per minute was determined using a scintillation counter.

### Animal Studies

All animal handling and treatment procedures were performed according to the approved Institutional Animal Care and Use Committee guidelines following the Association for Assessment and Accreditation of Laboratory Animal Care guidance. Cell line xenografts were performed in BALB/c nude mice except for the T47D model that used NSG mice implanted with 17-beta estradiol tablets (0.5 mg, 90-day release). PK/PD studies with xenograft tumors were established using standard protocols. PDX models were performed by XenoSTART (San Antonio, TX). Compound 1 and alpelisib were formulated in 30% 2-hydroxylpropyl-beta-cyclodextrin pH 8. Tissue western blots used standard protocols with snap-frozen tissues in radioimmunoprecipitation assay buffer with protease inhibitors. Primary antibodies pAKT (S473) (AB_2315049), AKT (AB_1147620), vinculin (AB_2728768) from Cell Signaling Technologies, and secondary antibodies: (IRDye 680CW Goat anti-Mouse IgG, AB_10956588), IRDye 800CW Goat anti-Rabbit IgG (AB_621843) from LiCOR were used. IHC samples were fixed in 10% NBF for 24 hours, transferred into 70% ethanol embedding, sectioning, staining, and quantification of tumor pAKT (Cell Signaling #4060). Plasma and tissue bioanalytical analysis of Compound 1 or alpelisib were measured following protein precipitation using liquid chromatography with tandem mass spectrometry. All methods and limits of quantification were adequate regarding specificity and sensitivity to support the PK analysis.

OGTT and ITT studies in BALB/c nude were carried out after 5 days of treatment. Food was removed for 5 hours, drug administered, and then 2 g/kg oral glucose (OGTT), or 0.75 U/kg intraperitoneal insulin (ITT) (Lilly Inc, France, #H1079), was administered 1 hour later. Blood glucose was measured at times indicated following tail vein collection or terminal bleed (One touch Glucose Meter, Roche, ACCU-CHEK Performa #06454038). Insulin was measured by ELISA (Crystal Chem, #90082). Metabolic profiling in CAL33 tumor-bearing mice was performed following a 4-hour fast, then dosed with drug for 1 hour before oral administration of 300-mg ¹³C-labeled glucose (Cambridge Isotope Labs #CLM-1396-0). Tissues were collected just prior to labeled glucose administration (0 hours) or after 30 minutes, snap-frozen, and analyzed (NYU metabolic core using Hybrid Metabolomics protocol, RRID:SCR_017935).

### Example 1: Isobolograms as a Measure of Combinatorial Benefit

### Determination of Constant Enzyme Activity as Determined by Varying Substrate an Inhibitor Concentrations.

Three models that measure synergy include:

1. Loewe additivity can define additivity as a drug combined with itself, with no interactions taking place. The Loewe additivity method is widely used, but assumes that the compounds have the same Ymax and hill slope.

2. The Bliss Independence model is based on concept of pharmacological independence.

3. The HSA model, which defines additivity as the max effect of the most potent compound is the combination. See Di Veroli, et al., Bioinformatics. 2016 Sep. 15; 32(18):2866-8.

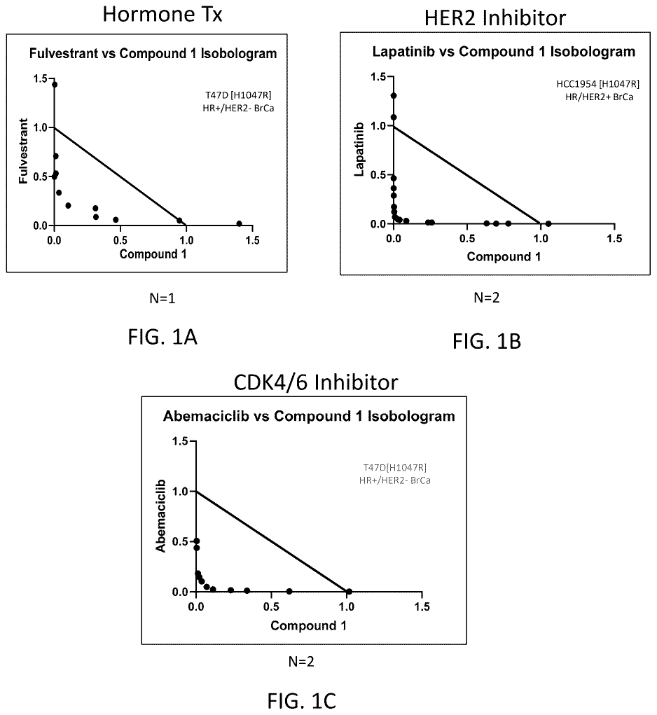

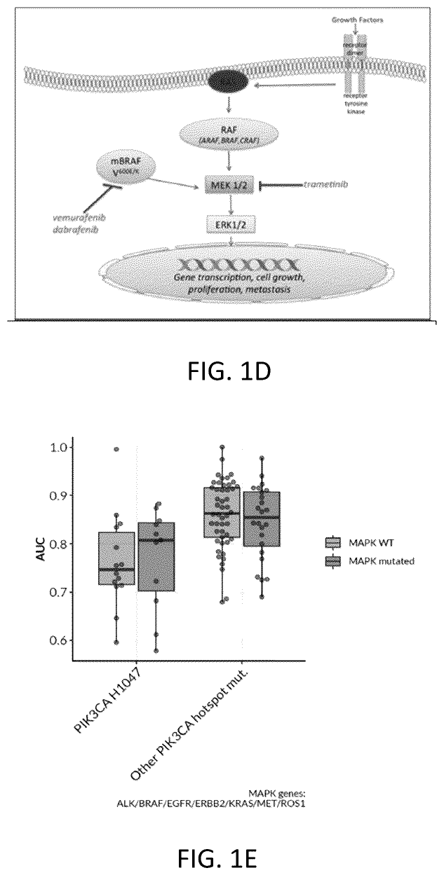

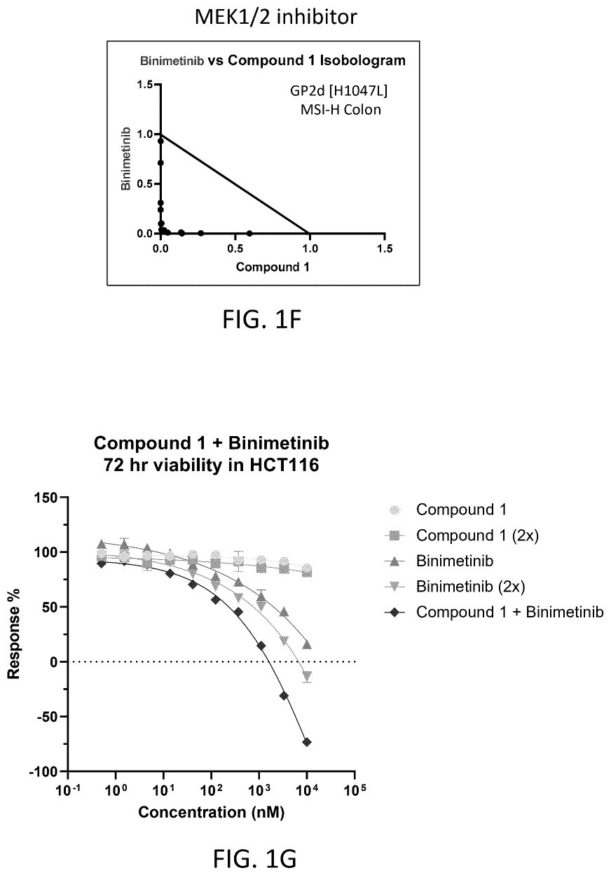

The quantifying and graphing synergy were as follows. Both the combination index and isobolograms stem from the Loewe additivity method. The calculation of the combination index (CI) using IC₅₀ quantified synergy. See, Altenburger, et. al., Handbook of Hazardous Materials. 1993, P. 15-27. A CI less than 0.75 indicates synergy, while a CI greater than 1.25 indicates antagonism. A CI between 0.75 to 1.25 is additive. Linear concentrations of the two drugs are on different axis, and the IC₅₀s of combinations are generally plotted. A linear isobole representing Loewe additivity runs between concentrations of equal value. See FIGS. 1A-1E.

### Example 2: Compound 1 in Combination with Fulvestrant Supports Clinical Benefit

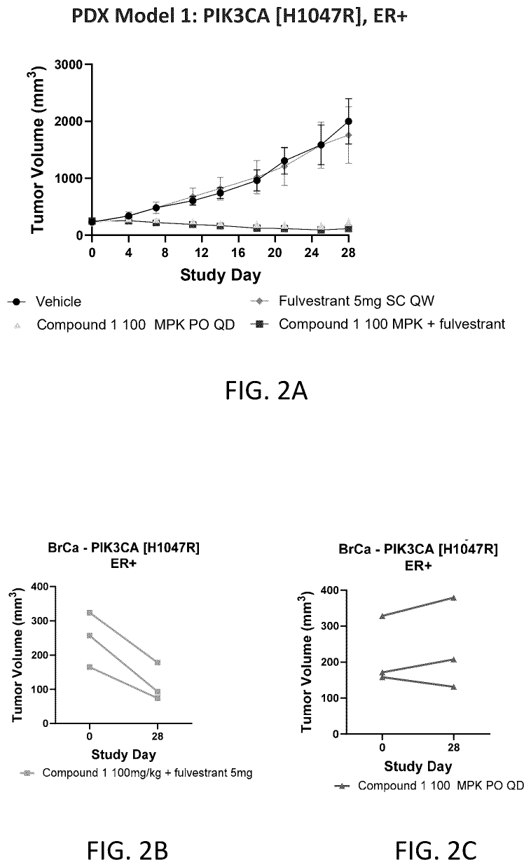

Strong tumor growth inhibition and stasis was observed with 100 mg/kg Compound 1. There was little to no growth inhibition with fulvestrant alone. Consistent tumor regressions were only observed with the combination of Compound 1 and fulvestrant. See FIGS. 2A-2C and Table 3.

TABLE 3

| Treatment | T/C |
|---|---|
| Vehicle | 100% |
| Compound 1 at 100 mg/kg QD | 5% |
| Fulvestrant at 5 mg/kg QW | 89% |
| Compound 1 at 100 mg/kg QD + | −33% |
| Fulvestrant at 5 mg/kg QW | |

### Example 3: Compound 1 in Combination with Palbociclib Supports Clinical Benefit

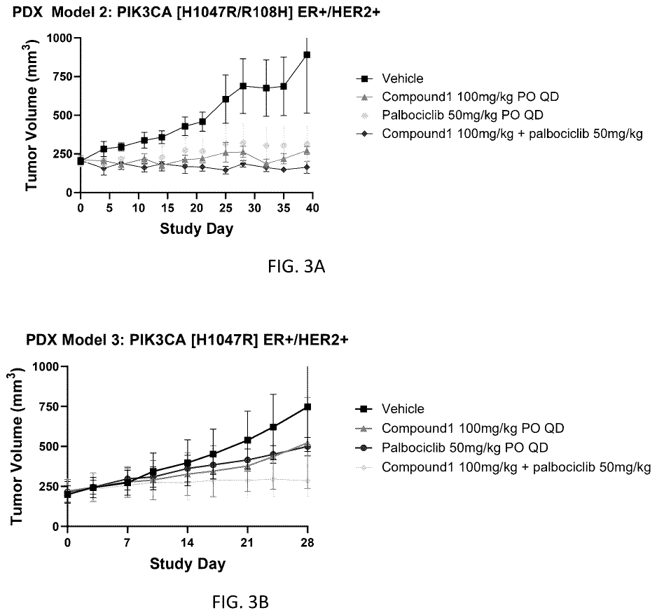

Strong tumor growth inhibition and stasis was observed with 100 mg/kg Compound 1 and with palbociclib alone. Tumor regressions were only observed with the combination of Compound 1 and palbociclib. See FIGS. 3A-3BC and Table 4.

TABLE 4

| Treatment | T/C |
|---|---|
| Vehicle | 100% |
| Compound 1 at 100 mg/kg QD | 9% |
| Palbociclib at 50 mg/kg QD | 19% |
| Compound 1 at 100 mg/kg QD + | −21% |
| Palbociclib at 50 mg/kg QD | |

### Example 4: CDX Model Study with GP21D Colon Adenocarcinoma Cells and Detroit562 Head and Neck Squamous Cell Carcinomas (HNSCCs) Cells

GP2D) and Detroit562 tumor cell lines are cultured and engrafted on to immunodeficient BALB/c nude mice. The mice are provided treatments as indicated in Tables 5 and 6.

TABLE 5

| GP2D | MAPK-driven COLO |
|---|---|
| Treatment | Dose |
| Vehicle | PO QD |
| Trametinib | 0.3 mg/kg PO |
| | QD |
| Compound 1 | 30 mg/kg |
| Compound 1 + | 30 mg/kg + |
| trametinib | 0.3 mg/kg |
| Compound 1 | 100 mg/kg |
| Compound 1 + | 100 mg/kg + |
| trametinib | 0.3 mg/kg |

TABLE 6

| Detroit562 | MAPK-driven HNSCC |
|---|---|
| Treatment | Dose |
| Vehicle | PO QD |
| Cetuximab | 5 mg/kg QW |
| | to 10 mg/kg |
| Compound 1 | 30 mg/kg |
| Compound 1 + | Same as |
| cetuximab | single agent |
| Compound 1 | 100 mg/kg |
| Compound 1 + | Same as |
| cetuximab | single agent |

### Example 5: PDX Model Study with ST1056 Breast Cancer Cells and ST433 Head and Neck Squamous Cell Carcinomas (HNSCCs) Cells

ST1056 tumor cell lines are biopsied and engrafted on to athymic nude, immunodeficient mice. Table 7 shows the study details. The mice are provided treatments as indicated in Table 8.

TABLE 7

| | |
|---|---|
| Animal Strain: | Athymic Nude, Immunodeficient Mice |
| Animal Age/Sex | 6-12 weeks/Female |
| sdafrgSSexSex: | |
| Selected Models: | ST1056 |
| Tumor Implantation: | SC Tumor Fragment (~70 mg) |
| Randomization: | Stratified by Mean Animal TV; 150-300 mm³ |
| Treatment Initiation: | Day 0 |
| Administration | 10 mL/kg; *Fixed-Volume |
| Volume: | |
| Control/ROA: | Compound 1 Vehicle/PO |
| Test Agent(s)/ROA: | Compound 1/PO |
| SOC/ROA: | *Fulvestrant/SC; Palbociclib/PO |
| Data Capture | Twice Weekly |
| Schedule: | |
| Study Type(s): | gTGI |
| Study Endpoint(s): | 1 cm³ or ≥28 Day |

TABLE 8

| | Compound 1 | |
|---|---|---|
| ST1056 [BrCa] | (mg/kg PO QD) | Dose of Other Agent(s) |
| Vehicle | — | — |
| Compound 1 | 30 | |
| Compound 1 | 100 | 100 mg/kg PO QD |
| Fulvestrant | — | *5 mg/mouse SC QW |
| Palbociclib | — | 50 mg/kg PO QD |
| Fulvestrant + Palbociclib | — | *5 mg SC QW/50 mg/kg PO QD |
| Compound 1 + Fulvestrant | 30 | *5 mg/mouse SC QW |
| Compound 1 + Palbociclib | 30 | 50 mg/kg PO QD |
| Compound 1 + Fulvestrant + | 30 | *5 mg SC QW/50 mg/kg PO QD |
| Palbociclib | | |
| Compound 1 + Fulvestrant | 100 | *5 mg/mouse SC QW |
| Compound 1 + Palbociclib | 100 | 50 mg/kg PO QD |
| Compound 1 + Fulvestrant + | 100 | *5 mg SC QW/50 mg/kg PO QD |
| Palbociclib | | |

*Administered at a fixed-volume dose.

ST433 tumor cell lines are biopsied and engrafted on to Fox Chase SCID (CB 17/Icr-Prkdcscid/IcrIcoCrl). Table 9 shows the study details and the mice are provided treatments as indicated in Table 10.

TABLE 9

| | |
|---|---|
| Animal Strain: | Fox Chase SCID Mice |
| Animal Age/Sex: | 6-12 weeks/Female |
| Selected Models: | ST443 |
| Tumor Implantation: | SC Tumor Fragment (~70 mg) |
| Randomization: | Stratified by Mean Animal TV; 150-300 mm³ |
| Treatment Initiation: | Day 0 |
| Administration | 10 mL/kg |
| Control/ROA: | Compound 1 Vehicle/PO |
| Test Agent(s)/ROA: | Compound 1/PO |
| SOC/ROA: | Cetuximab/IP |
| Data Capture | Twice Weekly |
| Study Type(s): | gTGI |
| Study Endpoint(s): | 2 cm³ or 28 Day |

TABLE 10

| ST443 [HNSCC] | Dose mg/kg | ROA/Schedule |
|---|---|---|
| Vehicle | — | PO/qd to end |
| Compound 1 at 100 mg/kg | 75 | PO/qd to end |
| Cetuximab at 0.25-10 mg/kg | 0.25-10 | IP/q7d to end |
| 1-3 times per week | | |
| Compound 1 + cetuximab at 5 | 75 | PO/qd to end |
| mg/kg QW to 10 mg/kg | 10 | IP/q7d to end |

### Example 6: Evaluation of the Anti-Tumor Effects of Compound 1 in Combination with Fulvestrant in T-47D Human Breast Cancer Xenograft Model in Immunodeficient Mice

Cell line T-47D was used for this ER⁺HER2⁻ CDX breast cancer model, T-47D (PI3Kα^{H1047R}) engrafted on immunodeficient mice.

Compound 1 (100 mg/kg) monotherapy caused robust tumor regression (141% TGI). Compound 1 in combination with fulvestrant (5 mg QW) was well-tolerated, with more consistent and deeper tumor regression. Table 11 shows that fulvestrant monotherapy (G2), or Compound 1 at a sub-maximal dose (50 mg/kg, G3) each yielded tumor growth inhibition, (52% and 92%, respectively), but together (G5), the combination demonstrated the benefits which exceeds either monotherapy alone, with 16% tumor regression.

Table 11 shows antitumor activity in T-47D xenografts. After 20 days of treatment, the mean tumor volume (TV) of the vehicle control group reached 413 mm³. Relative to the vehicle control, fulvestrant at 50 mg/kg QD and 5 mg/mouse s.c showed antitumor activity when analyzed by two-way RM ANOVA followed by Tukey post hoc comparisons of the means with TGI of 52% (P≤0.01), respectively.

Compound 1 at 50 mg/kg QD and 100 mg/kg QD, showed dose-dependent antitumor activity with the TGI value of 92% (P≤0.0001), and 141% (P≤0.0001), respectively. Compound 1 at 50 mg/kg QD combined with fulvestrant 5 mg/mouse s.c showed strong antitumor activity with the TGI value of 116% (P≤0.0001). Compound 1 at 100 mg/kg QD combined with fulvestrant 5 mg/mouse s.c a showed strong antitumor activity with the TGI value of 155% (P≤0.0001). Table 11 shows that G2, or G3 vs G5 demonstrated therapeutic synergy with tumor regression only seen in combination.

TABLE 11

| | | Tumor | Tumor | | | |
|---|---|---|---|---|---|---|
| | | Volume | Volume | ΔT/ | | |
| | | (mm³, | (mm³, | ΔC | TGI^{b} | P^{c} |
| Group | Treatment | PG-D0)^{a} | PG-D20)^{a} | (%) | (%) | Value |
| G1 | Vehicle | 182 ± 15 | 413 ± 40 | — | — | — |
| | qd × 20 days | | | | | |
| G2 | Fulvestrant 5 mg/mouse, | 182 ± 9 | 292 ± 24 | 48% | 52% | ** |
| | qw × 20 days | | | | | |
| G3 | Compound 1 | 182 ± 13 | 199 ± 15 | 8% | 92% | **** |
| | 50 mg/kg, qd × 20 days | | | | | |
| G4 | Compound 1 | 182 ± 12 | 88 ± 8 | −41% | 141% | **** |
| | 100 mg/kg, qd × 20 days | | | | | |
| G5 | Compound 1 50 mg/kg, PO | 181 ± 12 | 145 ± 23 | −16% | 116% | **** |
| | QD × 20 days + Fulvestrant | | | | | |
| | 5 mg/mouse, SC, , QW*20 | | | | | |
| G6 | Compound 1 100 mg/kg, | | | | | |
| | POQD × 20days + Fulvestrant | 182 ± 11 | 55 ± 6 | −55% | 155% | **** |
| | 5 mg/mouse, SC, QW × 20 | | | | | |

^{a}Mean ± SEM;
^{b}TGI = (1-T/C) × 100%, T/C = 100% × (TV treated final-TV treated initial)/(TV Vehicle final-TV Vehicle initial);
^{c}vs. Vehicle Control;
NS = No significant,
* P ≤ 0.05;
** P ≤ 0.01;
*** P ≤ 0.001;
**** P ≤ 0.0001 by two-way RM ANOVA followed by Tukey post hoc comparisons of the means.

### Example 7: Evaluation of Compound 1 as Monotherapy and in Combination with Other Antineoplastic Agents in Participants with Advanced Solid Tumor

This clinical trial is a multipart, open-label, phase 1/2 study evaluating the safety, tolerability, pharmacokinetics (PK), and preliminary antitumor activity of Compound 1 in participants with advanced solid tumors with certain mutations.

Part 1 will evaluate Compound 1 as monotherapy in participants with breast cancer and other solid tumor types; Part 2 will evaluate Compound 1 therapy as combination therapy with fulvestrant in participants with breast cancer.

Each study part will include a 28-day screening period, followed by treatment with Compound 1 monotherapy or combination therapy. Participants will remain in the study part to which they are initially enrolled throughout their participation in the study (ie, they will not move into other study parts).

TABLE 12
Arms and Interventions

| Arms | Assigned Interventions |
|---|---|
| Experimental: Part 1.1: Dose Escalation (Breast) | Drug: Compound 1 |
| Cohort A0: HR+/HER2− Breast cancer expressing PI3Kα | |
| H1047X mutations or other kinase domain mutations | |
| Experimental: Part 1.2-DE: Dose expansion at MTD | Drug: Compound 1 |
| Cohort A1: Gynecologic cancers | |
| Cohort A2: HNSCC | |
| Cohort A3: Other solid tumors not included in Cohorts | |
| A0, A1, or A2 | |
| Mutations for Cohorts A1, A2, and A3: PI3Kα H1047X | |
| mutations or other kinase domain mutations | |
| Cohort A4: Solid tumors expressing PI3Kα helical | |
| domain mutations (e.g., E542/E545) | |
| Experimental: Part 1.2-DS: RP2D Selection (Breast) | Drug: Compound 1 |
| Recommended Phase 2 dose (RP2D) | |
| Cohort A0: HR+/HER2− Breast cancer expressing PI3Kα | |
| H1047X mutations or other kinase domain mutations | |
| Experimental: Part 1.3: RP2D Expansion (Breast) | Drug: Compound 1 |
| Cohort A0: HR+/HER2− Breast cancer expressing PI3Kα | |
| H1047X mutations or other kinase domain mutations | |
| Experimental: Part 2.1: combination RP2D Selection | Fulvestrant will be administered |
| Cohort B: HR+/HER2− Breast cancer expressing PI3Kα | Drug: Compound 1 |
| H1047X mutations or other kinase domain mutations | according to local labeling. Other |
| | name: Faslodex |
| Experimental: Part 2.2: combination RP2D Expansion | Fulvestrant will be administered |
| Cohort B: HR+/HER2− Breast cancer expressing PI3Kα | Drug: Compound 1 |
| H1047X mutations or other kinase domain mutations | according to local labeling. Other |
| | name: Faslodex |

### Primary Outcome Measures

### Part 1.1 (Dose Escalation):

1. MTD: Number and proportion of participants who experienced at least 1 DLT during the first 28 days of treatment.

2. OBD: PK, pharnacodynamics, ORR, TEAEs/SAEs grade 2. Type, frequency, and severity of TEAEs according to CTCAE v5.0 criteria.

### Part 1.2 (Dose Selection) and Part 2.1:

1. PK, pharnacodynamics, ORR, and safety parameters

### Part 1.2 (Dose Expansion), Part 1.3 and Part 2.2:

1. ORR defined as the percentage of participants with PR or CR based on RECIST 1.1

### Selected Inclusion Criteria

1. Has an advanced or refractory solid tumor malignancy that is metastatic or locally advanced and unresectable

2. Has a new or recent tumor biopsy (collected at screening, if feasible) or archival tumor specimen within 12 months prior to screening

3. Has a tumor that harbors a documented PI3Kα mutation, as described herein, obtained either from tumor or plasma samples, determined by PCR or NGS-based assay as an FDA-approved test in US, or obtained as part of normal clinical care in a CLIA certified or similarly certified laboratory.

4. Has at least 1 measurable tumor lesion per RECIST 1.1

5. Is ≥18 years of age at the time of signing the ICF

6. Has an ECOG performance status score of 0 or 1 at screening

### Selected Exclusion Criteria

1. Has history (within ≤2 years before screening) of a solid tumor or hematological malignancy that is histologically distinct from the cancers being studied

2. Has symptomatic brain or spinal metastases

3. Has a tumor with mutations/deletions in PTEN and activating mutations in AKT or mTOR confirmed by a CLIA-certified or similarly certified laboratory, or has a tumor with mutations/deletions in PTEN and activating mutations in AKT (e.g., E17K) confirmed by a CLIA-certified or similarly certified laboratory.

4. Has an established diagnosis of diabetes mellitus type 1 or has uncontrolled diabetes mellitus type 2 requiring antihyperglycemic medication

5. Cohorts AG, A1, A2, A3, A4, and B: Has had prior treatment with PI3K/AKT/mTOR inhibitor(s), except in certain circumstances

6. Has had treatment with any local or systemic antineoplastic therapy or investigational anticancer agent within 14 days or 4 half-lives, whichever is longer, prior to the initiation of study treatment up to a maximum washout period of 28 days

7. Has toxicities from previous anticancer therapies that have not resolved to baseline levels or CTCAE grade≤1, with the exception of alopecia and peripheral neuropathy

8. Has had radiotherapy within 14 days before the initiation of study treatment

### Example 8. Compound 1 as a Mutant-Selective, Allosteric PI3Kα Inhibitor

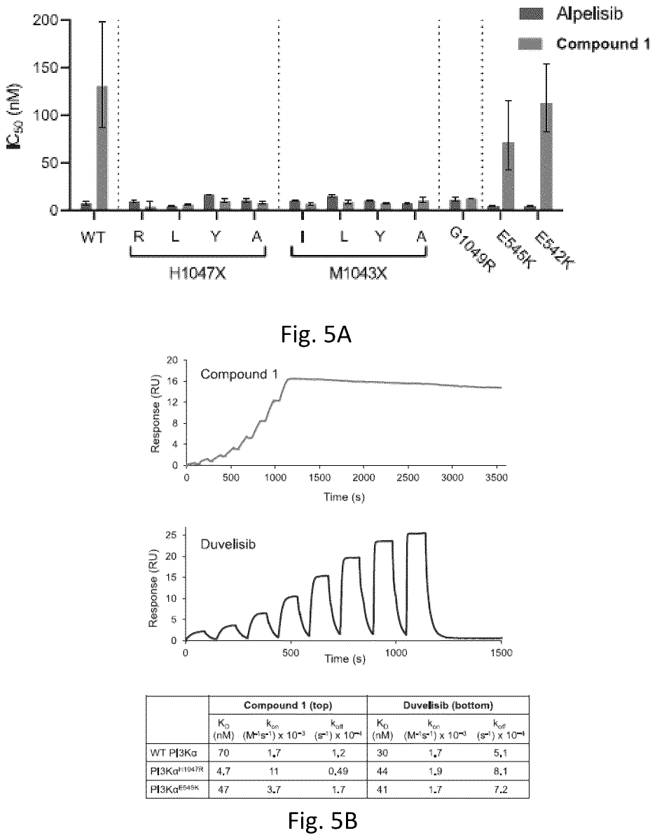

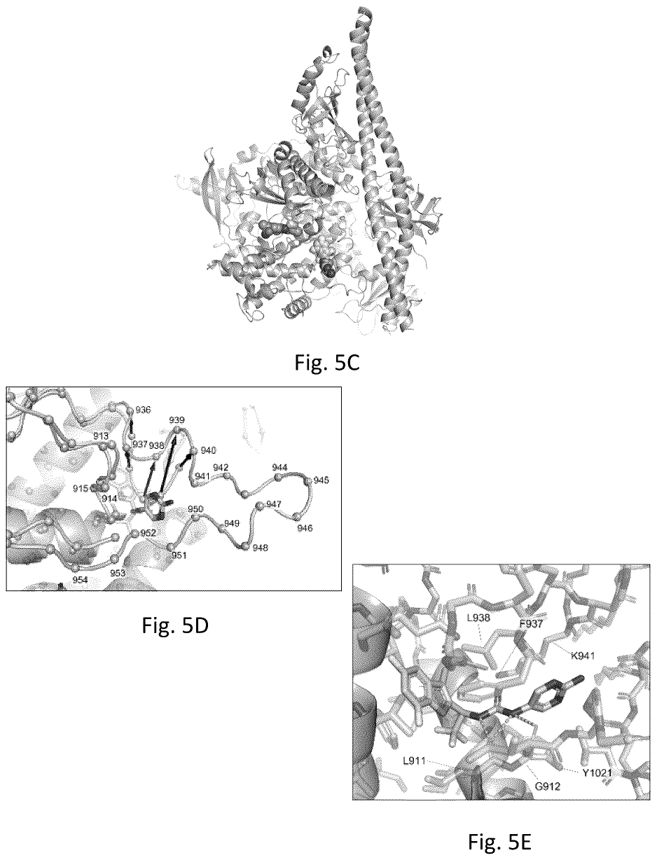

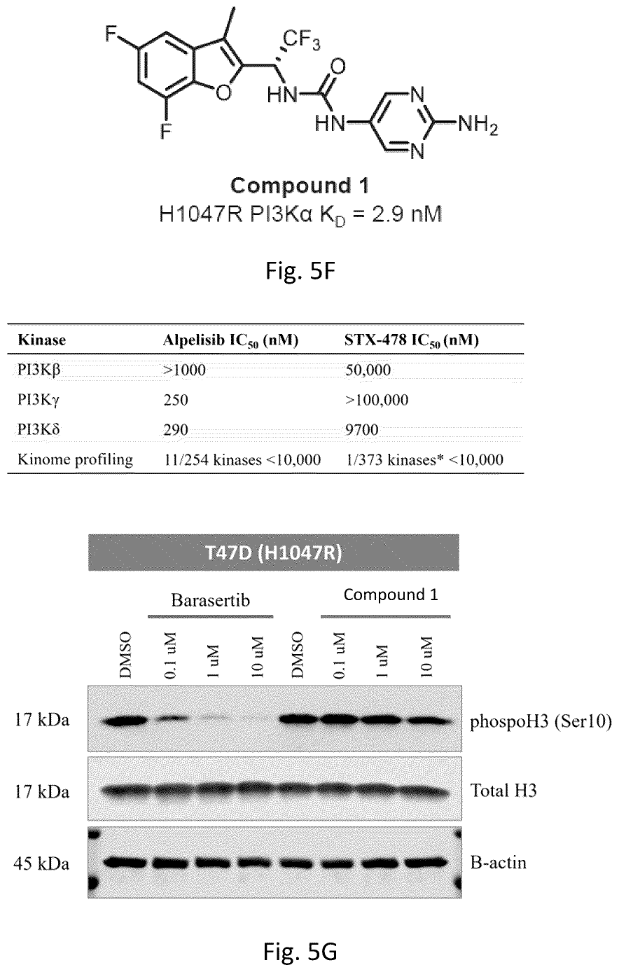

Compound 1 was confirmed to be a potent binder of H1047R PI3Kα by surface plasmon resonance (SPR; FIG. 5B). In addition to its low nanomolar binding affinity (equilibrium dissociation constant, K_{D}˜2.9 nM) for the H1047R mutant, Compound 1 had a 20-fold lower binding affinity for WT PI3Kα (K_{D}˜56 nM). In contrast, duvelisib (a non-selective, orthosteric PI3Kα inhibitor) showed approximately equal binding affinity for mutant and WT PI3Kα. Without being bound by theory, the H1047R mutant selectivity of Compound 1 may largely driven by a faster association constant (kₒₙ) and slightly slower dissociation constant (k_{off}) against H1047R versus WT PI3Kα (FIG. 5B), suggesting that the allosteric site occupied by Compound 1 may be more accessible in the H1047R-mutant form of the enzyme.

The biochemical potency and mutant selectivity of Compound 1 was compared with alpelisib in a panel of common oncogenic-mutant PI3Kα forms (FIG. 5A). Compound 1 was found to be a potent and selective inhibitor of all kinase-domain mutant PI3Kα forms found in cancer, including the most common variant H1047R (IC₅₀-9.4 nM), with 14-fold selectivity over WT PI3Kα (IC₅₀˜131 nM). Under these same assay conditions, Compound 1 had limited selectivity for hotspot helical-domain mutant (E542K/E545K) PI3Kα, while alpelisib showed no mutant selectivity as previously reported (Fritsch C, et al. Mol Cancer Ther 2014; 13(5):1117-29).

Compound 1 also demonstrated exquisite kinome-wide selectivity (FIG. 5G). A biochemical screen using 373 kinases representing approximately 70% of the human kinome, including PI3Kβ, PI3Kδ, and PI3Kγ isoforms, showed that only the AurB kinase was inhibited by >50% at 10 μM (IC₅₀=1.6 μM). Subsequent follow-up confirmed that Compound 1 showed limited AurB inhibition in cells at concentrations up to 10 μM.

Compared with published WT- and H1047R-PI3Kα x-ray crystal structures, the Compound 1 co-crystal structure revealed that Compound 1 occupies a novel allosteric site (FIG. 5C, Table 13) that forms due to a major conformational shift in residues 936-940, along with other smaller, local rearrangements (FIG. 5D). Specifically, residues F937 and L938 occupy positions that would directly clash with Compound 1. Rearrangement of side- and main-chain atoms of these residues resulted in repositioning of F937 and L938 to create space for the allosteric site. Additionally, relative to existing H1047R structures (3hhm and 3hiz; Mandelker D, et al. Proc Natl Acad Sci USA 2009; 106(40):16996-7001), the activation loop is better resolved in the Compound 1 co-structure. Compound 1 makes several specific protein contacts within the allosteric site (FIG. 5E).

### Example 9. Compound 1 Selectively Targets PI3Kα Activity and Cell Viability in PI3Kα-Mutant Cells

Compound 1 was studied and compared with alpelisib in a panel of ten human tumor cell lines harboring PI3Kα kinase-domain mutations (Table 15). In addition to H1047R, the panel included three cell lines carrying the second-most common kinase-domain mutation H1047L (EFM19, GP2D, and OAW42 cell lines), as well as three double-mutant forms (BT20, CAL148, and NCIH1048 cell lines). SKBR3 breast cancer cells are dependent on amplified WT PI3Kα activity for growth and were included as a comparator for WT selectivity (Cerami E, et al. Cancer Discov 2012; 2(5):401-4; Gao J, et al. Sci Signal 2013; 6(269):p 11).

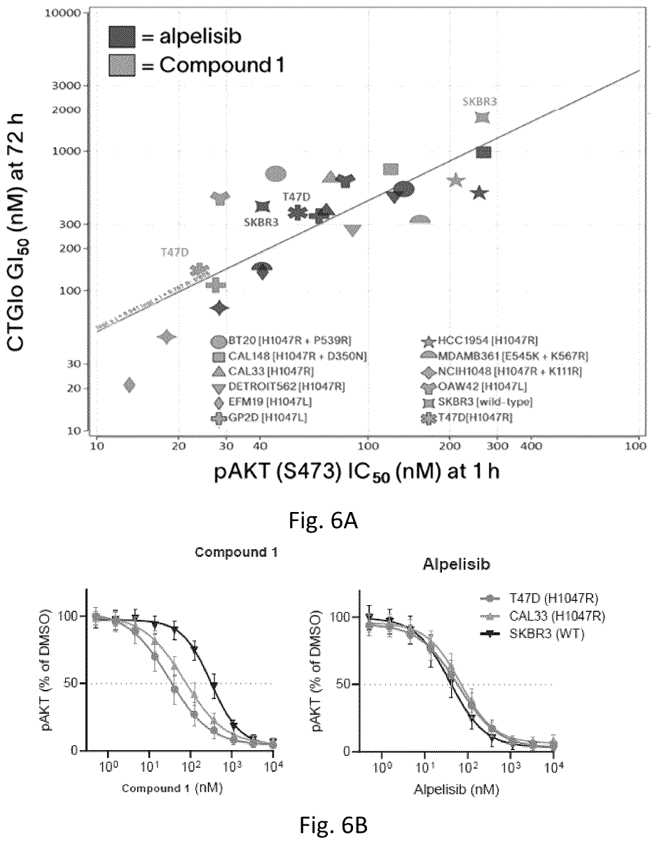

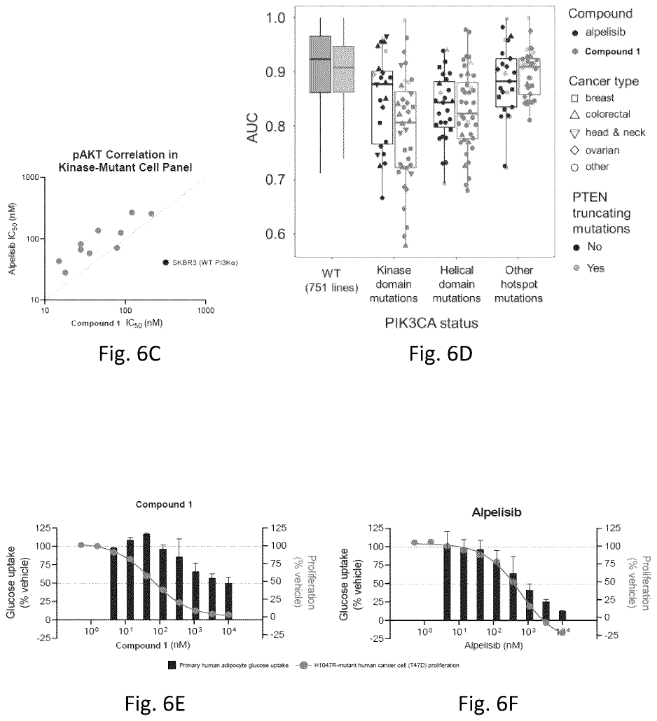

Target engagement was assessed with phosphorylated (serine 473) AKT (pAKT) as a marker of PI3Kα/AKT pathway activity. FIG. 6B shows Compound 1 and alpelisib dose response curves for PI3Kα kinase-domain mutant T47D and Cal33 cells and WT SKBR3 cells. Compound 1 effectively inhibited mutant-PI3Kα activity across the panel of cell lines, with IC₅₀ values ranging from ˜15 to ˜319 nM, which was similar to alpelisib IC₅₀ values of ˜28 to ˜268 nM (Table 15). Compound 1 was more potent than alpelisib in 9 of the 11 cell lines, the only exception being WT-PI3Kα-SKBR3 cells, which is expected based on the mutant selectivity of Compound 1 (correlation plot FIG. 6C, Table 15). In the ER⁺HER2⁻ breast cancer benchmark T47D (H1047R PI3Kα) cell line, Compound 1 was 9-fold more selective than in SKBR3 (WT PI3Kα) cells. In contrast, alpelisib showed no differentiation in selectivity between mutant- and WT-driven cell lines. When cell viability was studied in the same cell lines, there was a strong correlation between target engagement (pAKT) and cell viability (Pearson correlation coefficient=0.8 [log scale]) (FIG. 6A). These results confirmed that pAKT is a relevant translational biomarker for a mutant-selective inhibitor such as Compound 1.

TABLE 15
Compound 1 target engagement and cell viability activity in a human tumor cell
line panel.

| | | | Compound | Compound | | |
|---|---|---|---|---|---|---|
| | | | 1 | 1 | Alpelisib | |
| | | | pAKT | CTGlo | pAKT | Alpelisib |
| | | | HTRF | GMean | HTRF | CTGlo |
| | Genetic | | GMean | IC₅₀ | GMean | GMean |
| | PI3Kα | | IC₅₀ (nM) | (nM) | GI₅₀ (nM) | GI₅₀ (nM) |
| Cell line | status | RRID | (95% CI) | (95% CI) | (95% CI) | (95% CI) |
| BT20 | H1047R + | CVCL_0178 | 46 (27-79) | 654 (547-783) | 136 (58-321) | 516 (435-612) |
| | P539R | | | | | |
| CAL148 | H1047R + | CVCL_1106 | 121 (106-139) | 705 (555-895) | 268 (166-431) | 962 (774-1196) |
| | D350N | | | | | |
| CAL33 | H1047R | CVCL_1108 | 79 (67-92) | 577 (468-711) | 71 (65-77) | 340 (301-383) |
| DETROIT562 | H1047R | CVCL_1171 | 88 (63-123) | 246 (181-333) | 125 (79-199) | 436 (336-565) |
| EFM19 | H1047L | CVCL_0253 | 15 (13-16) | 20 (16-25) | 43 (37-50) | 127 (110-147) |
| GP2D | H1047L | CVCL_2450 | 28 (20-37) | 82 (53-127) | 66 (49-89) | 298 (218-408) |
| HCC1954 | H1047R | CVCL_1259 | 211 (177-252) | 457 (300-697) | 256 (210-313) | 384 (264-560) |
| NCIH1048 | H1047R + | CVCL_1453 | 18 (15-22) | 45 (38-53) | 28 (19-43) | 67 (48-94) |
| | K111R | | | | | |
| OAW42 | H1047L | CVCL_1615 | 28 (20-40) | 430 (342-541) | 82 (44-155) | 601 (529-682) |
| SKBR3 | WT | CVCL_0033 | 319 (290-351) | 1559 (1265-1921) | 41 (38-44) | 335 (285-394) |
| T47D | H1047R | CVCL_0553 | 36 (31-42) | 116 (88-151) | 58 (54-62) | 334 (299-372) |

GI₅₀, concentration of compound that reduces total cell growth by 50%;
GMean, geometric mean;
RRID, Research Resource Identifier.

Compound 1 activity was next evaluated in a high-throughput cell viability panel of approximately 900 tumor cell lines to identify markers of sensitivity. Consistent with the selectivity profile described above, cell lines with PI3Kα kinase-domain mutations were significantly more sensitive than WT cell lines (FIG. 6D). Unexpectedly, tumor cells with PI3Kα helical-domain mutations had nearly the same level of growth sensitivity to Compound 1, despite earlier results with recombinant proteins (FIG. 5E). The increased sensitivity of helical-domain mutant cell lines compared with WT PI3Kα cell lines may be explained by the dependence of mutant cell lines on PI3Kα for proliferation, whereas most WT cell lines are not. PI3Kα mutations located in hotspot helical and kinase domains appeared sensitive to Compound 1 (FIG. 6D). PTEN-inactivating mutations conferred resistance to PI3Kα inhibition in this study (P<0.0081), consistent with previous reports (Razavi P, et al. Nat Cancer 2020; 1(4):382-93).

The effect of Compound 1 on WT PI3Kα was assessed by evaluating insulin-mediated glucose uptake in human primary adipocytes (Hauner H, Int J Obes Relat Metab Disord 1998; 22(5):448-53). Differentiated primary human subcutaneous adipocytes were pre-treated with alpelisib or Compound 1, and then supplemented with [3H]-2-deoxy-glucose and 10 nM insulin. Alpelisib inhibited glucose uptake at concentrations as low as 100 nM, with nearly complete inhibition at 10 μM, whereas the concentration of Compound 1 needed to attain 50% inhibition (EC₅₀) was ≥10 μM (FIG. 6E). The maximum effect (Eₘₐₓ) of Compound 1 for glucose uptake was 38% whereas alpelisib caused deeper suppression with an Emax of 88%. An overlay of the cell viability dose-response curve obtained in T47D cells (FIG. 6A) illustrates the potentially improved therapeutic index of Compound 1 relative to alpelisib in relevant human-cell systems.

### Example 10. Compound 1 Treatment Yields Robust Anti-Tumor Efficacy in PI3Kα-Mutant Tumors Without Metabolic Dysregulation in Mice

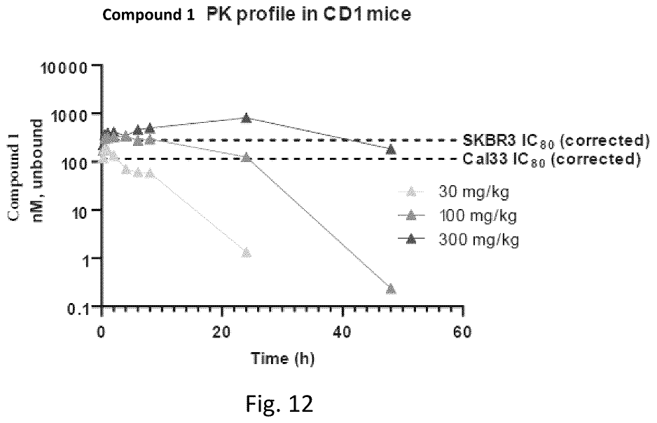

Pharmacological characterization of Compound 1 in vivo was designed to establish the metabolic safety and anti-tumor efficacy profile of Compound 1 compared with alpelisib. A 50 mg/kg once-daily (QD) dose of alpelisib was chosen as this was efficacious in published mouse xenograft models, albeit with noted glucose dysregulation (Fritsch C, et al. (2014) Mol Cancer Ther. 13(5): 1117-29). At this dose, the alpelisib plasma exposure (AUC) in mice (˜75,000 ng*hr/mL) exceeded the exposure of the maximum approved human dose by approximately two-fold (˜33,000 ng*hr/mL) (Juric D, et al. J Clin Oncol 2018; 36(13):1291-9). A lower dose (20 mg/kg) was also included. Based on the Compound 1 pharmacokinetic (PK) profile, 30- and 100-mg/kg QD doses were expected to bracket the 80% inhibitory concentration (IC₈₀) of relevant cancer cell lines, whereas the 300 mg/kg QD dose surpassed these levels (FIG. 12).

With regards to metabolic control, the primary consequence of WT PI3Kα inhibition is to block insulin action (e.g., insulin resistance), impairing glucose disposal and causing hyperglycemia (Fruman D A, Chiu H, Hopkins B D, Bagrodia S, Cantley L C, Abraham R T. The PI3K pathway in human disease. Cell 2017; 170(4):605-35; James D E, Stockli J, Birnbaum M J. The aetiology and molecular landscape of insulin resistance. Nat Rev Mol Cell Biol 2021; 22(11):751-7).

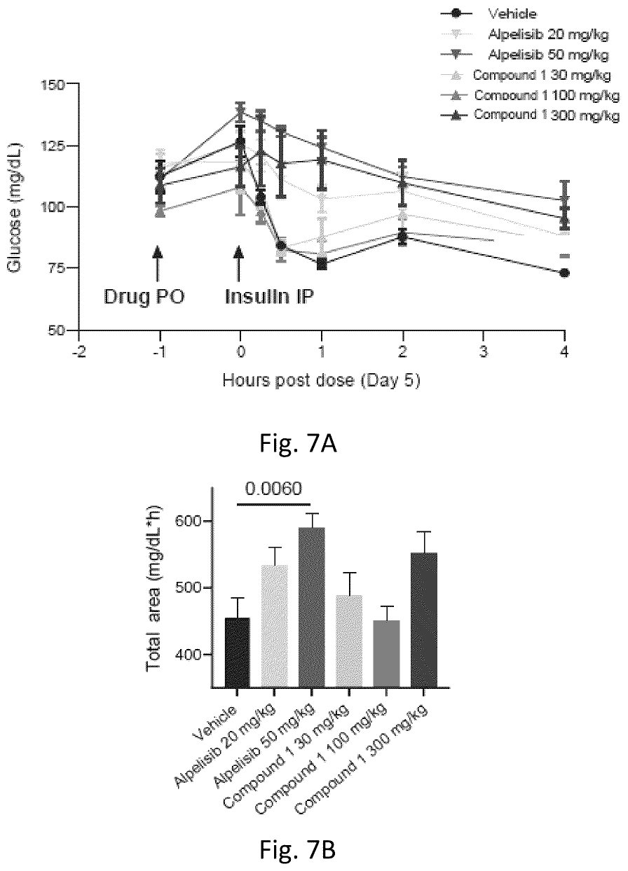

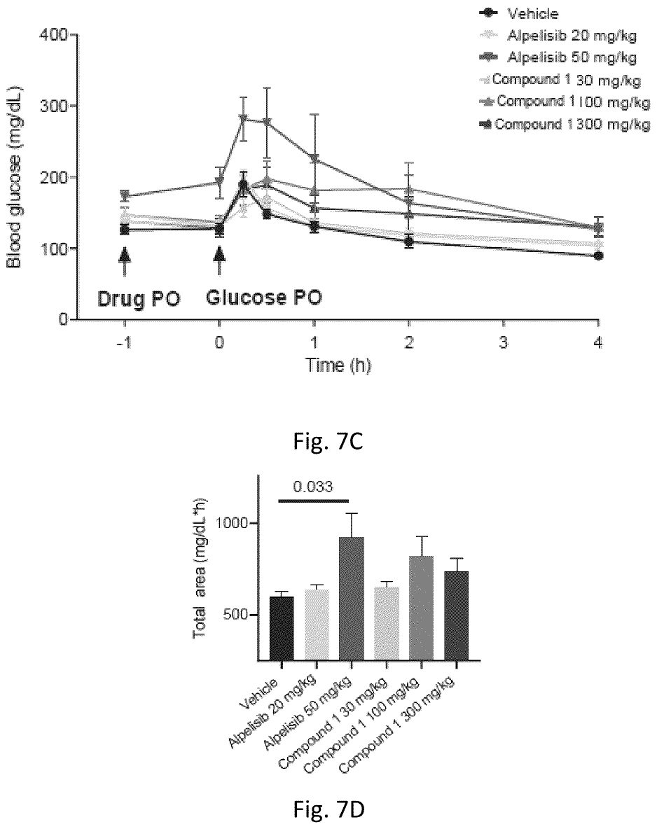

The impact of alpelisib and Compound 1 doses on insulin sensitivity was analyzed using an insulin tolerance test (ITT) and oral glucose tolerance test (OGTT), following 5 days of repeat dosing in non-tumor-bearing female, BALB/c nude mice, the sex/strain frequently used in xenograft studies. Alpelisib treatment resulted in a dose-dependent reduction in glucose disposal in both the ITT and OGTT consistent with insulin resistance (FIGS. 7A/7B and 7C/7D, respectively). In contrast, Compound 1 treatment was not associated with significant changes in glucose AUC, although there was a non-statistically significant increase at the 300 mg/kg dose in the ITT. Notably, Compound 1 had no effect on body weight or fasting plasma glucose after 5 days of treatment. These studies established that repeat doses of Compound 1 at 100 mg/kg QD were well-tolerated without metabolic dysregulation, whereas alpelisib caused overt insulin resistance at 50 mg/kg dose levels.

### Example 11. Benchmarking Compound 1 in the Cal33 (H1047R PI3Kα) Human HNSCC Xenograft Model

The Cal33 (H1047R PI3Kα) head and neck squamous cell carcinoma (HNSCC) cell model was selected to benchmark therapeutic activity and pharmacodynamic biomarkers in vivo, since this cell line showed intermediate sensitivity to PI3Kα inhibition in culture (FIG. 6A). The study consisted of three arms: an efficacy group in which animals received test articles for 28 days; a PK/pharmacodynamic (PK/PD) group in which animals received Compound 1 or alpelisib treatment for 3 days; and a group in which tumor-bearing animals received an oral bolus of [U-¹³C]-glucose to assess the effects of alpelisib and Compound 1 on glucose uptake and glucose oxidation in target tissues.

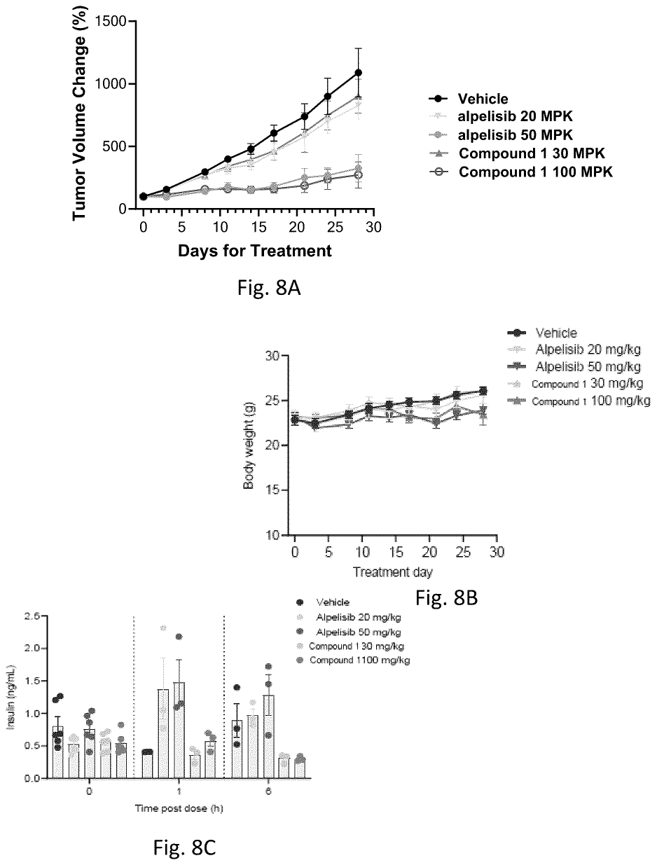

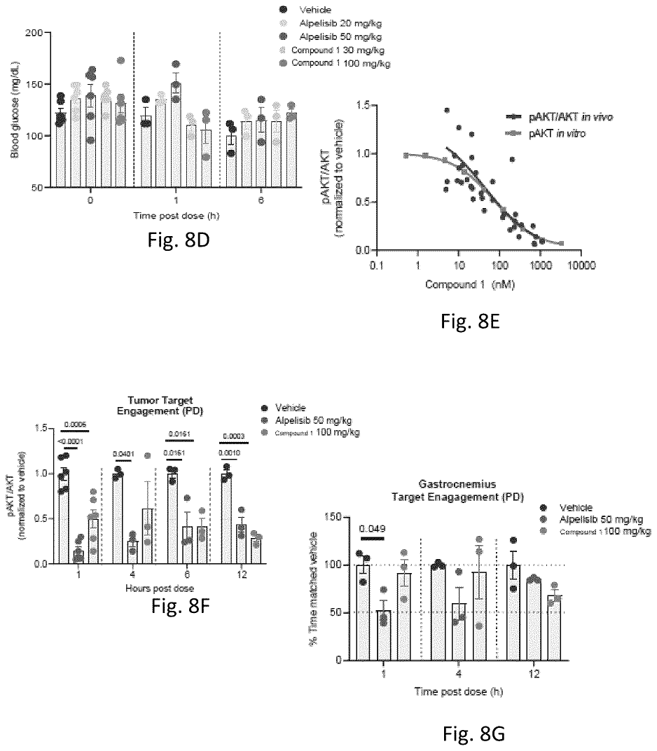

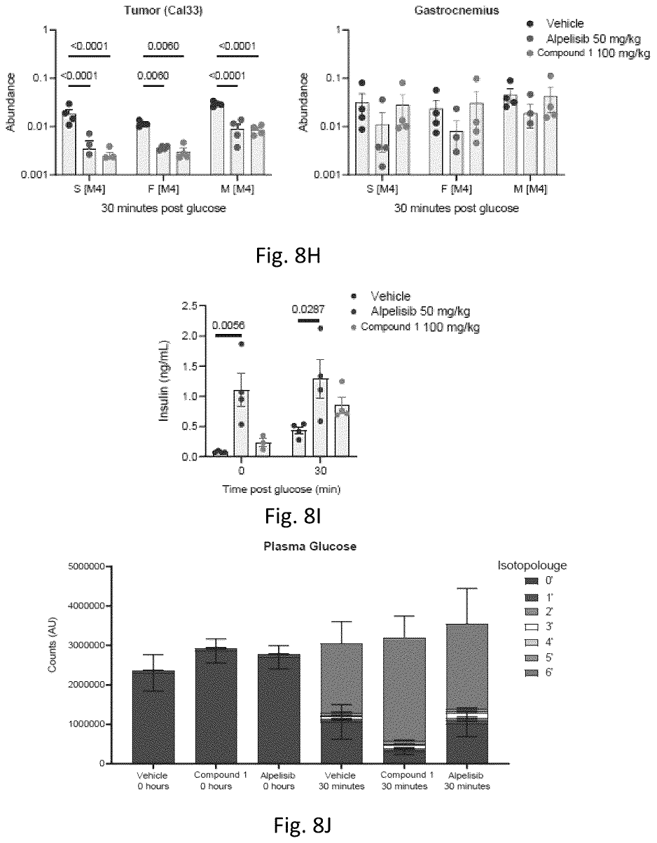

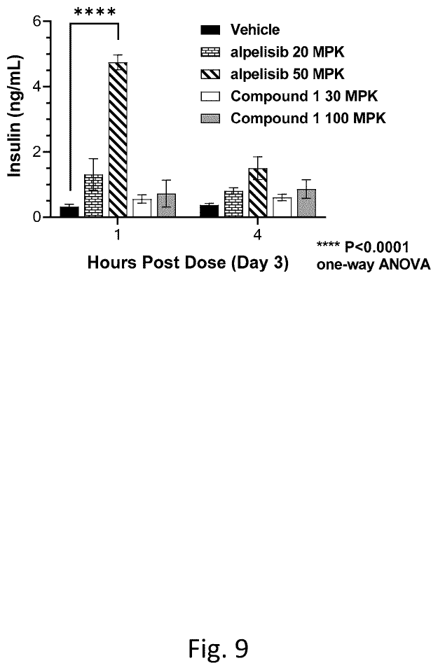

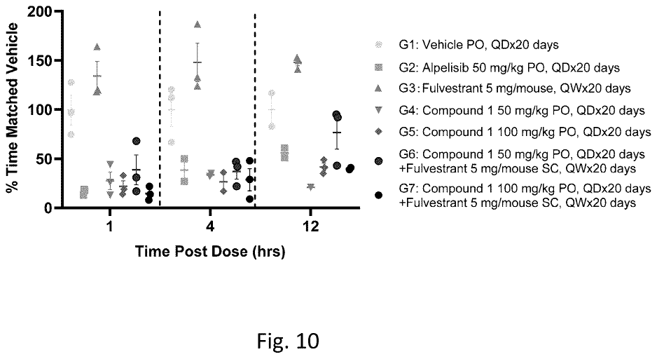

In the efficacy arm, there was a dose-dependent reduction in tumor volume with both compounds; Compound 1 at 30 mg/kg showed efficacy similar to alpelisib 20 mg/kg, and Compound 1 at 100 mg/kg showed efficacy similar to alpelisib at 50 mg/kg (FIG. 8A). Both compounds were well tolerated at all doses, with no change in body weight (FIG. 8B). Alpelisib treatments raised serum insulin at 1-hour post dose on Day 28 (P=0.0585, FIG. 8C and FIG. 9), with a similar trend in glucose (P<0.083, FIG. 8D). In all subsequent cell-line derived xenograft (CDX) studies, alpelisib 50 mg/kg caused a significant increase in serum insulin at 1-hour post dose while Compound 1 at 100 mg/kg did not (Table 16).

TABLE 16
Summary of CDX studies: tumor growth inhibition
and insulin levels (1-hour post-dose).

| | | | Compound 1 | | Alpelisib | |
|---|---|---|---|---|---|---|
| Cell line | | | 100 mg/kg QD | | 50 mg/kg QD | |
| (day of | | | | Δ | | Δ |
| final | Cancer | PI3Kα | TGI | Insulin^{b} | TGI | Insulin^{b} |
| dose) | type | mutation | (%)^{a} | (ng/ml) | (%)^{a} | (ng/mL) |
| Cal33 | HNSCC | H1047R | 82**** | +1.07^{NS} | 79**** | +1.07^{NS} |
| (Day 28) | | | | | | |
| Detroit 562 | HNSCC | H1047R | 92**** | +0.21^{NS} | 96**** | +2.33** |
| (Day 22) | | | | | | |
| GP2D | Colon | H1047L | 2**** | +0.03^{NS} | 79**** | +1.59**** |
| (Day 28) | | | | | | |
| NCI-H1048 | Lung | H1047R + | 85**** | +0.37^{NS} | 87**** | +1.21** |
| (Day 23) | | K11IR | | | | |
| HCC1954 | Breast | H1047R | 69* | +0.33^{NS} | 77** | +2.38** |
| (Day 28) | | | | | | |
| T-47D^{d} | Breast | H1047R | 52**** | +0.28^{NS} | 100**** | +0.79**** |
| (Day 20) | | | | | | |

Tumor growth inhibition or
^{a}regression (negative TGI) relative to Day 1 dosing.
^{b}One-hour post dose.
*P ≤ 0.05,
**0.01,
***0.001,
****0.0001.
Negative TGI values indicate % regression.
^{NS}not significant;
NSG, NOD scid gamma.

PD biomarkers of target engagement (pAKT/AKT ratio) and Compound 1 tumor drug levels were measured 1-, 4-, and 12-hours post dose on Day 3 (PK/PD group), and 1- and 6-hours post final dose (Day 28). The curve-fit relationship between tumor-drug concentration and pAKT/AKT levels in Cal33 tumor xenografts and in vitro is shown in FIG. 8E. The calculated IC₅₀ from this curve fit was ˜45 nM in tumor compared with ˜18 nM from cell culture when corrected for matrix binding (Pearson r correlation coefficient=−0.613; P<0.0001), thereby demonstrating a convincing target engagement in vitro:in vivo correlation. In this xenograft study, tumor growth inhibition (TGI) of 82% in the Compound 1 100 mg/kg QD dose group was associated with average pAKT suppression of 57% (treated/vehicle AUC₁₋₁₂ₕ); and for the alpelisib 50 mg/kg QD group, a TGI of 79% was associated with average pAKT suppression of 66% (FIG. 8F). Unlike alpelisib, Compound 1 did not reduce skeletal muscle pAKT/AKT (FIG. 8G).

Inhibition of PI3Kα is known to suppress glucose metabolism in tumor and host tissues (Hopkins B D, et al. Nature 2018; 560(7719):499-503; Juric D, et al. (2018) J Clin Oncol 36(13): 1291-9; Dockx Y, et al. Mol Imaging 2021; 2021:5594514; Sarker D, et al. Clin Cancer Res 2015; 21(1):77-86).

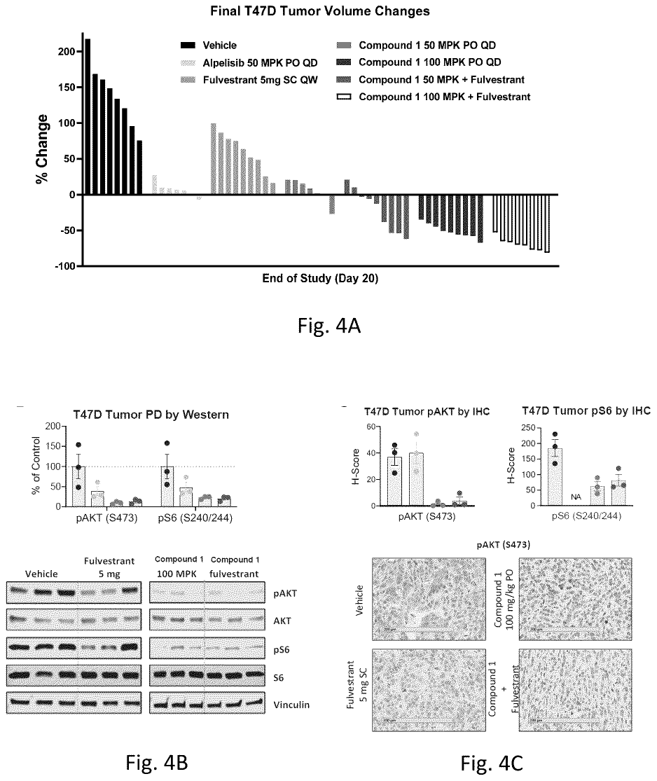

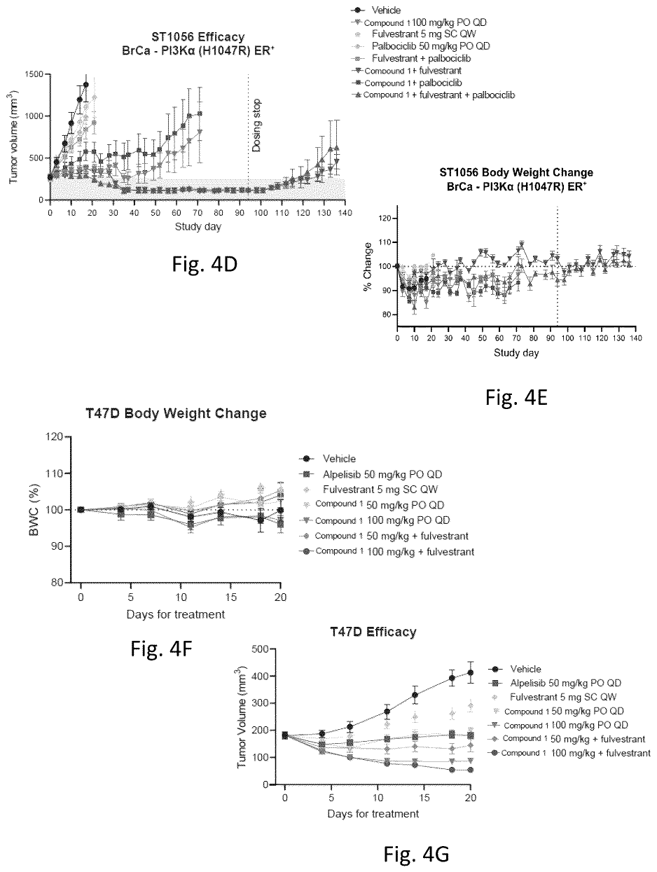

To assess the effects of alpelisib and Compound 1 on this process, Cal33 tumor-bearing mice were pre-treated with drug or vehicle then given U-¹³C-glucose by oral gavage. After 30 minutes, the tumor and skeletal muscle metabolites were extracted and quantified using liquid chromatography-mass spectrometry (Lopes M, et al. Cell Rep 2021; 37(2):109833). The oral bolus led to robust labelling of over 70% of circulating glucose carbon in all groups (FIG. 8J). In tumors, both alpelisib and Compound 1 significantly reduced the incorporation of ¹³C- into TCA intermediates, a marker of glucose oxidation (FIG. 4H). However, only alpelisib-treated mice demonstrated reductions in glucose oxidation in the skeletal muscle (FIG. 8H). This reduction occurred despite higher levels of circulating insulin in this group (30 min alpelisib vs vehicle), suggesting insulin resistance in this tissue (FIG. 8I). These data support Compound 1 selectively inhibits mutant PI3Kα but not the WT enzyme found in host tissues.

### Example 12. Compound 1 Is Efficacious Across a Panel of PI3Kα-Mutant CDX and PDX Tumors, without Evidence of Insulin Resistance

This detailed metabolic characterization of Compound 1 and activity in the Cal33 xenograft model established that the optimal Compound 1 dose was 100 mg/kg QD and demonstrated that Compound 1 was more efficacious than a clinically matched dose of alpelisib (20 mg/kg). Therefore, Compound 1 at 100 mg/kg was carried forward into a panel of PI3Kα-mutant CDX and patient-derived xenograft (PDX) models representative of several cancers. Models included colon cancer (GP2D), lung cancer (NCIH1048), HNSCC (Detroit 562), and HR⁻HER2⁺ breast cancer (HCC1954). High-dose alpelisib (50 mg/kg QD) was included as a benchmark and all studies were conducted in BALB/c nude mice.

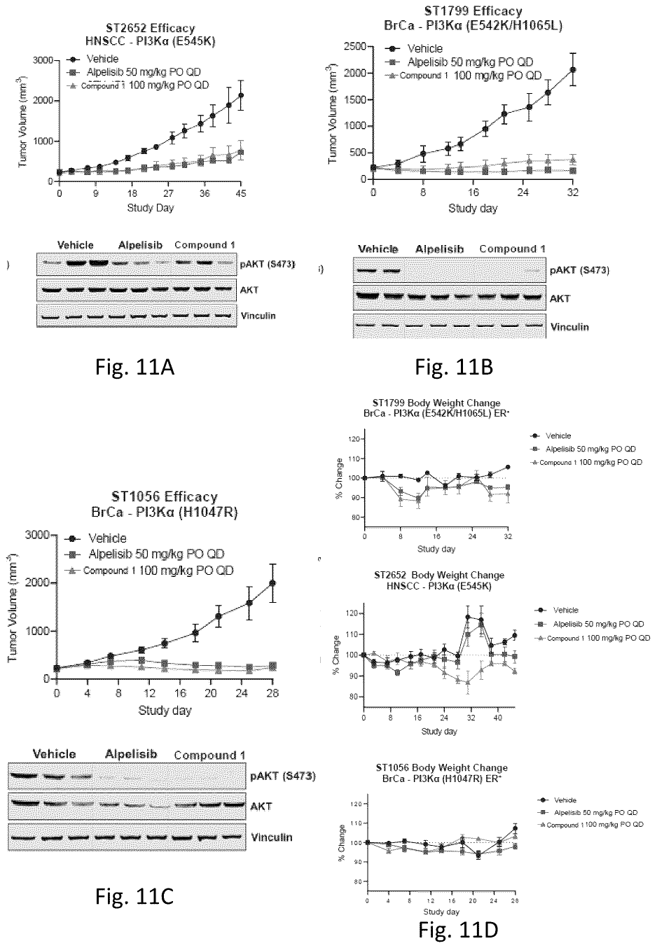

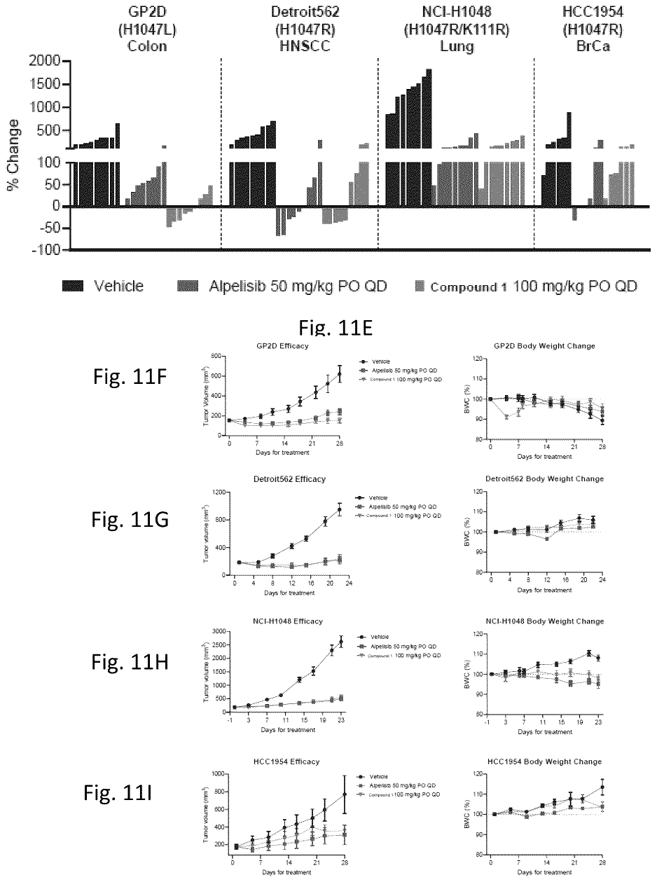

The key efficacy endpoint was TGI or regression, and tolerability measures included body weight and insulin levels at 1-hour post dose at end of study. No treatment-specific effects on body weight were noted with either Compound 1 or alpelisib (FIGS. 11F-11I). The TGI of Compound 1 at 100 mg/kg QD treatment was similar or superior to 50 mg/kg QD alpelisib treatment (FIG. 11E, Table 17). Compound 1 demonstrated robust efficacy in GP2D tumor xenografts that express H1047L PI3Kα, the second-most prevalent kinase domain mutation. The waterfall plot shows tumor regressions in half of the Compound 1 treated animals, while no regression was seen with alpelisib treatment (FIG. 11E). In the Detroit562 HNSCC model, robust efficacy was seen with both compounds, including tumor regressions in 5 of the 9 study animals. Similarly, Compound 1 and alpelisib treatment provided comparable tumor growth control in NCIH1048 lung carcinoma, which contains a double (H1047R/K411R) PI3Kα mutation, as well as in the HCC1954 HR⁺HER2⁺ breast cancer model (FIG. 11E). In every study described above, significant increases in serum insulin were observed in animals dosed with alpelisib 50 mg/kg 1-hour post dose, while Compound 1 at 100 mg/kg was not associated with elevated insulin (Table 17). While both alpelisib and Compound 1 treatment caused significant TGI, HCC1954 cells showed the lowest response of all cell lines tested in the CDX panel. HCC1954 cells were also a weaker responder to both agents in cell culture (FIG. 6A) and may reflect reduced efficacy of PI3Kα inhibitor monotherapy in HER2⁺ cancers, which is thought to be related to compensatory HER3 activation (Serra V, et al. Oncogene 2011; 30(22):2547-57; Chandarlapaty S, et al. Cancer Cell 2011; 19(1):58-71).

TABLE 17
Summary of CDX studies: tumor growth inhibition
and insulin levels (1-hour post-dose).

| | | | Compound 1 at | | Alpelisib | |
|---|---|---|---|---|---|---|
| Cell line | | | 100 mg/kg QD | | 50 mg/kg QD | |
| (day of | | | | Δ | | Δ |
| final | Cancer | PI3Kα | TGI | Insulin^{b} | TGI | Insulin^{b} |
| dose) | type | mutation | (%)^{a} | (ng/mL) | (%) | (ng/mL) |
| Cal33 | HNSCC | H1047R | 82**** | +1.07^{NS} | 79**** | +1.07^{NS} |
| (Day 28) | | | | | | |
| Detroit 562 | HNSCC | H1047R | 92**** | +0.21^{NS} | 96**** | +2.33** |
| (Day 22) | | | | | | |
| GP2D | Colon | H1047L | 2**** | +0.03^{NS} | 79**** | +1.59**** |
| (Day 28) | | | | | | |
| NCI-H1048 | Lung | H1047R + | 85**** | +0.37^{NS} | 87**** | +1.21** |
| (Day 23) | | K111R | | | | |
| HCC1954 | Breast | H1047R | 69* | +0.33^{NS} | 77** | +2.38** |
| (Day 28) | | | | | | |
| T-47D^{d} | Breast | H1047R | 52**** | +0.28^{NS} | 100**** | +0.79**** |
| (Day 20) | | | | | | |

Tumor growth inhibition or
^{a}regression (negative TGI) relative to Day 1 dosing.
^{b}One-hour post dose.
*P ≤ 0.05,
**0.01,
***0.001,
****0.0001.
Negative TGI values indicate % regression.
^{NS}not significant;
NSG, NOD scid gamma.

Compound 1 and alpelisib monotherapies were evaluated in two breast cancer PDX models, ST1056 (kinase-domain mutant [H1047R]) and ST1799 (a double mutant, E542K/H1065L). A third HNSCC xenograft model (ST2652) carrying only a helical-domain mutation (E542K) was also evaluated. Compound 1 was highly efficacious in all three models, including the (E542K) helical-domain mutant, and was generally similar to alpelisib (FIGS. 11A-11C). For both Compound 1 and alpelisib, a reduction in pAKT/AKT was seen 4-hours post dose in all models (FIGS. 11A-11C).

### Example 13. Safety and Efficacy of Clinically Relevant Combination Therapies

Compound 1 and fulvestrant monotherapies and in combination was evaluated in the T47D cell xenograft model. In the ER⁺HER2⁻ PDX model, Compound 1, fulvestrant, and palbociclib monotherapies and pairwise and triple combinations of these compounds were assessed.

The T47D cell line represents an important benchmark for the mutant PI3Kα mechanism, and is a well-established estrogen-dependent, ER⁺HER2⁻ breast cancer xenograft model, previously characterized with alpelisib treatment (Fritsch C, et al. (2014) Mol Cancer Ther 13(5): 1117-29). Compound 1 monotherapy demonstrated dose-dependent TGI, with the 50 mg/kg QD dose achieving TGI similar to high-dose alpelisib (50 mg/kg QD) and the Compound 1 at 100 mg/kg QD dose yielding significant tumor regression in every animal (FIG. 4A). Fulvestant monotherapy provided only ˜50% TGI and adding either the 50 or 100 mg/kg doses of Compound 1 led to regression in the majority of the xenografts (20% and 70% regression, respectively). Fulvestrant monotherapy and Compound 1 combinations were well tolerated based on body weight (FIGS. 4F-4G). Single-dose PK/PD measures were performed in tumors collected 4 hours after administration of Compound 1 at 100 mg/kg and 24 hours after fulvestrant. Phospo-AKT and pS6, biomarkers of PI3Kα pathway activity, were modestly reduced by fulvestrant, whereas Compound 1 led to significant inhibition as assessed by Western blot and IHC (FIGS. 4B and 4C, respectively).

Combination studies, including the triple combinations of Compound 1 with palbociclib and fulvestrant, were extended to an aggressive ER⁺HER2⁻ breast cancer PDX model. ST1056 tumors grew rapidly in the vehicle, palbociclib monotherapy, fulvestrant monotherapy, and fulvestrant and palbociclib combination groups requiring animals to be removed from study on day 17 (FIG. 4D). Compound 1 led to durable tumor growth suppression in the majority of animals for 49 days or more. The addition of palbociclib to Compound 1 provided no additional efficacy, but was well tolerated until Day 71, when the group was terminated. The most remarkable responses occurred with the combination of Compound 1 and fulvestrant, and the triple combination of Compound 1, fulvestrant and palbociclib. Tumor suppression continued between Day 28 and Day 94 in every animal in both groups. Treatment was stopped on Day 94 and modest regrowth was only observed after a month off of treatment when the study ended (FIG. 4D). Compound 1 monotherapy, Compound 1 in combination with fulvestrant and the triple combination including palbociclib were all well-tolerated based on body weight changes (FIG. 4E).

1. A method of treating breast cancer in a subject in need thereof, comprising administering to the subject
a compound of the formula 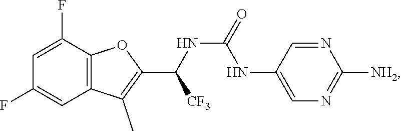 [SMILES: Cc1c([C@@H](NC(=O)Nc2cnc(N)nc2)C(F)(F)F)oc2c(F)cc(F)cc12]
or a pharmaceutically acceptable salt thereof;
a selective estrogen receptor modulator or degrader (SERM/SERD); and
a CDK4/6 inhibitor.

2.-15. (canceled)

16. The method of claim 1, wherein the SERM/SERD is clomifene, cyclofenil, broparoestrol, ormeloxifene, raloxifene, toremifene, lasofoxifene, bazedoxifene, ospemifene, enclomiphene, serophene, tamoxifen, fulvestrant, elacestrant, camizestrant, rintodestrant, clotrimazole, palazestrant, or fenticonazole.

17. The method of claim 1, wherein the SERM/SERD is fulvestrant.

18. The method of claim 1, wherein the CDK4/6 inhibitor is ribociclib.

19. The method of claim 1, wherein the CDK4/6 inhibitor is palbociclib.

20. The method of claim 1, wherein the CDK4/6 inhibitor is abemaciclib.

21.-37. (canceled)

38. The method of claim 1, wherein the breast cancer is HER2⁺ breast cancer.

39. The method of claim 1, wherein the breast cancer is HER2⁻ breast cancer.

40. The method of claim 1, wherein the breast cancer is ER⁺ breast cancer.

41. The method of claim 1, wherein the breast cancer is triple negative breast cancer.

42.-53. (canceled)

54. The method of claim 1, wherein the breast cancer is ER+, HER2− breast cancer.

55. The method of claim 1, wherein the breast cancer is metastatic or locally advanced.

56. The method of claim 1, wherein the SERM/SERD is fulvestrant and the CDK4/6 inhibitor is palbociclib.

57. The method of claim 1, wherein the SERM/SERD is fulvestrant and the CDK4/6 inhibitor is ribociclib.

58. The method of claim 1, wherein the SERM/SERD is fulvestrant and the CDK4/6 inhibitor is abemaciclib.

59. The method of claim 1, wherein the SERM/SERD is imlunestrant and the CDK4/6 inhibitor is palbociclib.

60. The method of claim 1, wherein the SERM/SERD is imlunestrant and the CDK4/6 inhibitor is ribociclib.

61. The method of claim 1, wherein the SERM/SERD is imlunestrant and the CDK4/6 inhibitor is abemaciclib.

62. A method of treating PIK3CA-mutated, hormone receptor (HR)-positive, human epidermal growth factor receptor 2 (HER2)-negative, locally advanced or metastatic breast cancer in a subject in need thereof, comprising administering to the subject:
a compound of the formula 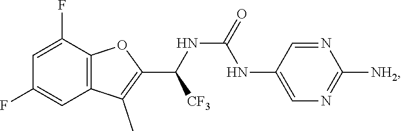 [SMILES: Cc1c([C@@H](NC(=O)Nc2cnc(N)nc2)C(F)(F)F)oc2c(F)cc(F)cc12]
or a pharmaceutically acceptable salt thereof;
a selective estrogen receptor modulator or degrader (SERM/SERD); and
a CDK4/6 inhibitor.

63. The method of claim 62, wherein the SERM/SERD is fulvestrant and the CDK4/6 inhibitor is palbociclib.

64. The method of claim 62, wherein the SERM/SERD is fulvestrant and the CDK4/6 inhibitor is ribociclib.

65. The method of claim 62, wherein the SERM/SERD is fulvestrant and the CDK4/6 inhibitor is abemaciclib.

66. The method of claim 62, wherein the SERM/SERD is imlunestrant and the CDK4/6 inhibitor is palbociclib.

67. The method of claim 62, wherein the SERM/SERD is imlunestrant and the CDK4/6 inhibitor is ribociclib.

68. The method of claim 62, wherein the SERM/SERD is imlunestrant and the CDK4/6 inhibitor is abemaciclib.
